(19) United States
(12) Patent Application Publication
(10) Pub. No.: US 2026/0191999 A1
(43) Pub. Date: Jul. 9, 2026

(54) COMPOUNDS AND THEIR USE

(71) Applicant: KING'S COLLEGE LONDON, London (GB)

(72) Inventors: Timothy H. Witney, London (GB); Muhammet Tanç, London (GB)

(21) Appl. No.: 19/128,395

(22) PCT Filed: Nov. 8, 2023
(86) PCT No.: PCT/EP2023/081108

§ 371 Date: May 8, 2025

(30) Foreign Application Priority Data
Nov. 9, 2022 (GB) 2216665.6

(51) Int. Cl. A61K 51/04; A61P 35/00; C07B 59/00; C07D 401/12; C07F 5/02; C07F 7/22

(52) U.S. Cl. CPC A61K 51/0455; A61P 35/00; C07B 59/002; C07D 401/12; C07F 5/025; C07F 7/2208; C07B 2200/05

(57) ABSTRACT

The present invention pertains generally to the field of diagnostic and therapeutic compounds. More specifically the present invention pertains to certain radiolabelled compounds, their precursors, and the respective uses. The radiolabelled compounds, inter alia, bind ALDH and emit radiation for detection by molecular imaging or for treatment of a disease mediated by ALDH.

### RELATED APPLICATIONS

The present application claims priority to, and the benefit of, GB 2216665.6 filed 9 Nov. 2022 (09/11/2022), the contents of which are incorporated herein by reference in their entirety.

### TECHNICAL FIELD

The present invention pertains generally to the field of diagnostic and therapeutic compounds. More specifically the present invention pertains to certain radiolabelled PQM-H compounds and their precursors (referred to herein as “PQM-H compounds” and “PQM-P compounds” respectively). The PQM-H compounds, inter alia, bind ALDH, more specifically ALDH1A1, and emit radiation for detection by molecular imaging or for treatment of a disease mediated by ALDH.

The present invention also pertains to pharmaceutical compositions comprising such PQM-H compounds, and the use of such compounds and compositions:
to bind, e.g., inhibit ALDH, both in vitro and in vivo, especially to bind, e.g., inhibit, ALDH1A1;
for molecular imaging, e.g., via the molecular imaging modalities: positron emission tomography (PET), single-photon emission computed tomography (SPECT), and scintigraphy; especially PET and/or SPECT imaging of: cancer; drug-resistant cancer; disorders associated with ALDH1A1 overexpression; and
to treat disorders including cancer and drug resistant cancer.

The present invention also pertains to the use of such PQM-P compounds in the preparation of the PQM-H compounds; processes for preparing PQM-H compounds from PQM-P compounds; and kits comprising PQM-P compounds.

### BACKGROUND

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

### Aldehyde Dehydrogenase (ALDH)

Aldehyde dehydrogenases (ALDHs) are a family of enzymes that catalyze the NAD(P)+ dependent oxidation of a wide variety of aldehydes to their corresponding carboxylic acids (Singh et al., 2013). There are currently 20 known functional human ALDHs (Black et al. 2009) that mediate the metabolism of aldehydes generated during oxidative stress (Vassilou et al., 2000; Hartley et al., 1995), amino acid and biogenic amine metabolism (Ambroziak et al., 1991), retinoic acid biosynthesis (Duester et al., 2003), and ethanol metabolism (Vassilou et al., 2000). ALDHs control the detoxification of exogenous reactive aldehydes and therapeutic drugs such as cyclophosphamide (Sladek, 2003).

Aberrant expression of ALDH is associated with many diseases, including cancer, with increased expression and activity of ALDH shown to be a predictor of metastatic potential and poor overall survival (Marcato et al., 2011). In particular, the ALDH1A1 isozyme is a well-characterized marker of cancer stem cells, which are known for their tumor-initiating properties and resistance to conventional therapy (Nakahata et al., 2015; Raha et al., 2014; Liu et al., 2013).

Studies have shown that resistance to chemotherapy and poor prognosis is associated with high ALDH1A1 activity in breast (Liu et al., 2014), ovarian (Landen et al., 2010), prostate (Li et al., 2010), colon (Kahlert et al., 2012), and lung (Huang et al., 2013) cancer. As a consequence, ALDH1A1 has been considered as a target for anti-cancer therapy, with ALDH inhibitors shown to reverse chemoresistance in a range of preclinical tumor models (Abdullah, 2013).

Given the causal link between ALDH1A1 expression and chemoresistance in some cancers, the identification of high ALDH1A1-expressing tumours represents a clinical challenge that if solved, could significantly improve patient outcomes.

Imaging these tumours using ALDH1A1 expression as a marker of chemoresistance could inform therapeutic interventions and offer the chance to tailor bespoke patient treatments (Greenwood et al., 2021).

Imaging strategies for evaluating ALDH activity have, to the most part, been restricted to fluorescence-based assays in isolated cells (Anorma et al., 2018; Maity et al., 2017; Minn et al., 2014; Ucar et al., 2009). However, poor tissue penetration of the fluorescent signal and requirement for direct tumour injection currently limits in vivo utility.

Other methods to detect cancer therapy resistance are disadvantageous because they are invasive, e.g., requiring tumor biopsy (Woolston et al., 2021), or require extensive data analysis (Shen et al., 2021; Volm et al., 2015).

To circumvent these limitations, PET has been proposed as an alternative to fluorescence-based imaging (Pereira et al., 2019; Vaidyanathan et al., 2009).

### Known Compounds

To date, radiotracer focussed imaging strategies for ALDH1A1 have used a substrate-based approach, that is, to design chemical probes having:
a) an aldehyde that can serve as a substrate for ALDH1A1;
b) a radioisotopic atom that would allow for detection via gamma counting/PET imaging;
c) a suitable hydrophobic-hydrophilic balance to allow passive diffusion across cell membranes; and
d) corresponding oxidised products which are sufficiently polar so as to be retained within the cells (i.e., unable to passively diffuse out of a cell).

Such substrate-based radiotracers have been reported to provide advantages such as increased sensitivity with respect to radiolabelled inhibitors due to the possibility for substrate-based agents to become trapped inside a target cell after conversion of the aldehyde to a carboxylic acid (Pereira et al., 2019).

Vaidyanathan et al., 2009, describes the two following iodinated ALDH1A1 substrates:

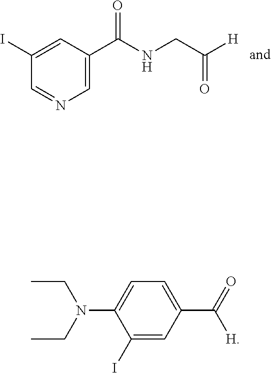

WO 2011/087823 A1 describes certain fluoro- and iodo-containing aldehydes of the following structural formula as ALDH substrates for use as diagnostic imaging agents or as therapeutic agents:

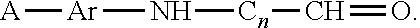

WO 2013/048811 A1 describes certain detectably labelled ALDH substrates of the following structural formula, which are allegedly useful for in vivo imaging and treatment of cancer:

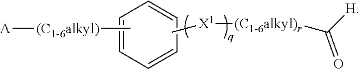

WO 2014/145493 A1 describes certain radioactive detectable substrates for ALDH of the following structural formula, which are allegedly useful for selecting cells positive for ALDH:

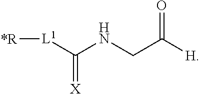

Pereira et al., 2019, describes the development of certain fluorinated ALDH1A1-selective chemical probes, including the following compound:

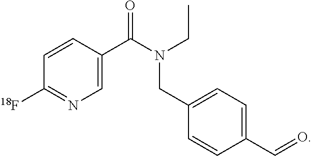 [SMILES: CCN(Cc1ccc(C=O)cc1)C(=O)c1ccc([18F])nc1]

Pereira et al., 2022, describes the development of the following acylal prodrug of the compound described in Pereira et al. 2019 (and shown above):

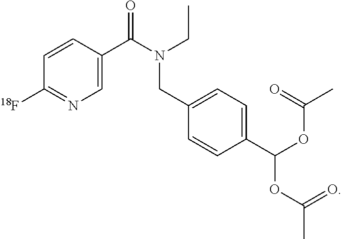 [SMILES: CCN(Cc1ccc(C(OC(C)=O)OC(C)=O)cc1)C(=O)c1ccc([18F])nc1]

Substrate-based imaging agents have shown promise in vitro. However, in vivo, substrate-based imaging agents have been found to suffer from poor half-life in blood (Pereira et al., 2019).

Aldehyde substrates were found to be rapidly oxidised to carboxylic acids before reaching target cells, thereby preventing diffusion across cell membranes and retention in ALDH expressing cells, even when protected via a prodrug strategy. No radiolabelled ALDH substrate has yet been shown to provide differentiation between drug-sensitive and drug-resistant tumors, in vivo (Pereira et al., 2022).

Fluorescent compounds have been considered as an alternative to radiolabelled compounds for detecting activity of ALDH (Minn et al. 2013; Yuen et al., 2016; Maity et al., 2017; Anorma et al., 2018; Yagishita et al., 2021; Okamoto et al., 2022).

Such fluorescent compounds have limited utility because scattering of emitted light by bodily tissue reduces the scenarios in which fluorescence is a viable means of imaging. For example, such compounds can be used in vitro, on outer bodily surfaces, or to image deeper tissues in conjunction with invasive techniques.

In contrast, radiotracer focussed imaging strategies carry the significant benefit of permitting whole-body clinical imaging.

### Potency/Selectivity

The PQM-H compounds described herein are highly potent ALDH1A1 binders, e.g., inhibitors, that are also highly selective for ALDH1A1, for example, as compared to ALDH2.

### SUMMARY OF THE INVENTION

One aspect of the invention pertains to certain radiolabelled compounds according to formula (I), as described herein (referred to herein as PQM-H compounds).

Another aspect of the invention pertains to certain precursor compounds to the PQM-H compounds, according to formula (Ia), as described herein (referred to herein as PQM-P compounds).

Another aspect of the invention pertains to the use of a PQM-P compound in the preparation of a PQM-H compound.

Another aspect of the invention pertains to a process for preparing a PQM-H compound from a PQM-P compound, as described herein.

Another aspect of the invention pertains to a kit comprising a PQM-P compound and instructions to prepare a PQM-H compound.

Another aspect of the invention pertains to a pharmaceutical composition comprising a PQM-H compound and a pharmaceutically acceptable carrier, diluent, or excipient.

Another aspect of the invention pertains to a PQM-H compound or a pharmaceutical composition comprising a PQM-H compound, for use in diagnosis or therapy.

Another aspect of the invention pertains to a PQM-H compound or a pharmaceutical composition comprising a PQM-H compound, for use in the diagnosis or treatment of cancer.

In one embodiment, the diagnosis or treatment is diagnosis or treatment of drug-resistant cancer.

Another aspect of the invention pertains to a method of medical imaging comprising the steps:
1) administering a PQM-H compound or a pharmaceutical composition comprising a PQM-H compound to a subject; then
2) imaging the subject by PET, SPECT or scintigraphy As will be appreciated by one of skill in the art, features and preferred embodiments of one aspect of the invention will also pertain to other aspects of the invention.

### BRIEF DESCRIPTION OF THE DRAWINGS

FIG. 1a: Graph showing cisplatin-induced growth inhibition in SKOV3-ip1 cells (white circles) and SKOV3-TRip2 cells (filled squares) using an MTT assay 72 h post treatment (n=3-5).

FIG. 1b: Bar chart showing relative ALDH1A1 mRNA expression in SKOV3-ip1 cells and SKOV3-TRip2 cells (n=2).

FIG. 1c: Image of Western blot reading for ALDH1A1 protein expression in SKOV3-ip1 cells and SKOV3-TRip2 cells.

FIG. 1d: Graphs showing ALDH-specific retention of the Aldefluor reagent in the presence and absence of the ALDH inhibitor N,N-diethyl amino benzaldehyde (DEAB).

FIG. 1e: Bar chart showing the ratio of Aldefluor median fluorescence intensity (MFI) in DEAB and vehicle-treated SKOV3-ip1 and SKOV3-TRip2 cells (n=3). **, P<0.01. Data are mean±SD.

FIG. 2a: Graph showing the inhibition of ALDH1A1 activity in the presence of PQM-C-001, (squares, IC₅₀=0.015 μM) and DEAB (circles, IC₅₀=0.298 μM).

FIG. 2b: Graph showing the inhibition of ALDH2 activity in the presence of PQM-C-001, (squares, IC₅₀>100 μM) and DEAB (circles, IC₅₀=1.19 μM).

FIG. 2c: Bar chart showing uptake of PQM-H-001, denoted [¹⁸F]PQM-H-001, by drug-resistant SKOV3-TRip2 cells (filled squares/white bars on right) and cisplatin sensitive SKOV3-ip1 cells (white circles/filled bars on left) at 20, 40, and 60 min.

FIG. 2d: Bar chart showing uptake of PQM-H-002, denoted [¹²⁵I]PQM-H-002, by drug-resistant SKOV3-TRip2 cells (filled squares/white bars on right) and cisplatin sensitive SKOV3-ip1 cells (white circles/filled bars on left) at 20, 40, and 60 min.

FIG. 3a: Representative time course axial PET/CT images of PQM-H-001, in BALB/c-nu/nu mice with SKOV3-ip 1 (top row) and SKOV3-TRip2 (bottom row) tumours. The tumour is denoted by the dotted circles.

FIG. 3b: Representative PET/CT maximum intensity projections of PQM-H-001 in BALB/c-nu/nu mice bearing SKOV3-ip1 (left) and SKOV3-TRip2 (right) tumours. The tumour is denoted by the dotted circles.

FIG. 4: Graph showing time versus radioactivity curves for PQM-H-001 injected into BALB/c-nu/nu mice bearing SKOV3-ip1 (circles) and SKOV3-TRip2 (squares) tumors, normalised to the percentage injected activity. Data expressed as the mean±standard deviation. n=3-4 animals per group.

FIG. 5: Bar chart showing ex vivo biodistribution of PQM-H-001 (denoted [¹⁸F]PQM-H-001) in organs of interest of BALB/c-nu/nu mice bearing SKOV3-ip 1 (white bars on left of each pair) and SKOV3-TRip2 (filled bars on right of each pair) ovarian cancer tumours. Data expressed as the mean±standard deviation. n=3-4 animals per group.

FIG. 6: HPLC co-elution chromatogram trace for PQM-H-001 (denoted [¹⁸F]PQM-H-001) and non-radioactive reference compound (denoted PQM-C-001); the taller peak in grey corresponds to PQM-H-001; the smaller peak in black corresponds to reference compound PQM-C-001.

FIG. 7: HPLC co-elution chromatograph trace for PQM-H-002 (denoted [¹²⁵I]PQM-H-002) and non-radioactive reference compound (denoted PQM-C-002); the taller peak in grey corresponds to PQM-H-002; the smaller peak in black corresponds to reference compound PQM-C-002 FIG. 8: HPLC co-elution chromatograph trace for PQM-H-007 (denoted [¹²⁵I]PQM-H-007) and non-radioactive reference compound PQM-C-007; the taller peak corresponds to PQM-H-007; the smaller peak corresponds to reference compound PQM-C-007.

FIG. 9: HPLC co-elution chromatograph trace for PQM-H-017 (denoted [¹²⁵I]PQM-H-017) and non-radioactive reference compound PQM-C-017; the taller peak corresponds to PQM-H-017; the smaller peak corresponds to reference compound PQM-C-017.

FIG. 10a: Graph showing uptake of PQM-H-002 by A549-WT (positive ALDH1A1) (filled circles) and A549-KO-ALDH1A1 (negative for ALDH1A1) (white circles) human lung cancer cells; at 20, 40, 60, and 120 min.

FIG. 10b: Graph showing uptake of PQM-H-002 by HEK293-WT (negative for ALDH1A1) (black circles) and HEK293-ALDH1A1 (positive for ALDH1A1) (grey circles) human embryonic kidney cells, at 20, 40, 60, and 120 min.

FIG. 11a: Graph showing uptake of PQM-H-007 by A549-WT (positive ALDH1A1) (filled circles) and A549-KO-ALDH1A1 (negative for ALDH1A1) (white circles) human lung cancer cells; at 20, 40, 60, and 120 min.

FIG. 11b: Graph showing uptake of PQM-H-007 by HEK293-WT (negative for ALDH1A1) (black circles) and HEK293-ALDH1A1 (positive for ALDH1A1) (grey circles) human embryonic kidney cells, at 20, 40, 60, and 120 min.

FIG. 12a: Graph showing uptake of PQM-H-017 by A549-WT (positive ALDH1A1) (filled circles) and A549-KO-ALDH1A1 (negative for ALDH1A1) (white circles) human lung cancer cells; at 20, 40, 60, and 120 min.

FIG. 12b: Graph showing uptake of PQM-H-017 by HEK293-WT (negative for ALDH1A1) (black circles) and HEK293-ALDH1A1 (positive for ALDH1A1) (grey circles) human embryonic kidney cells, at 20, 40, 60, and 120 min.

FIG. 13: Graph showing uptake of PQM-H-022 by A549-WT (positive ALDH1A1) (filled circles) and A549-KO-ALDH1A1 (negative for ALDH1A1) (white circles) human lung cancer cells; at 20, 40, 60, and 120 min.

### DETAILED DESCRIPTION OF THE INVENTION

### Compounds

One aspect of the present invention relates to certain compounds which are related to quinoline:

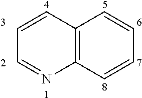

More specifically, the compounds are related to piperazin-1-yl(3-quinolyl)methanone (“PQM”):

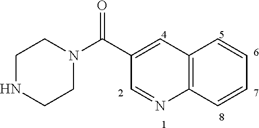

Furthermore, compounds described herein have:
(a) a substituent at the 4-position (denoted herein as —R⁴ or —R¹⁴);
(b) a substituent at the secondary amine of the piperazine in PQM (denoted herein as -L¹-R^{L} or -L¹¹-R^{1L});
(c) one or two substituents at the 6-, 7-, or 8-positions (denoted herein as —R⁶, —R⁷, and —R⁸; or —R¹⁶, —R¹⁷, and —R¹⁸);

Thus, one aspect of the present invention is a compound of the following formula, or a pharmaceutically acceptable salt, hydrate, or solvate thereof, wherein —R⁴, -L¹-, —R^{L}, —R⁶, —R⁷ and —R⁸ are as defined herein (for convenience, collectively referred to herein as “PQM-H” compounds”):

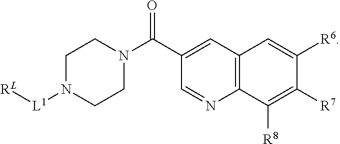

The PQM-H compounds described herein are radiolabelled compounds. That is, the PQM-H compounds are enriched with a radioisotope at one substituent position.

For the avoidance of doubt, in a population of PQM-H compounds, the substituent that is a radioisotope comprises said radioisotope in an abundance that is greater than the natural abundance of the radioisotope.

Some embodiments include the following:

### Radiolabelled Compounds

(1) A radiolabelled compound according to formula (I):

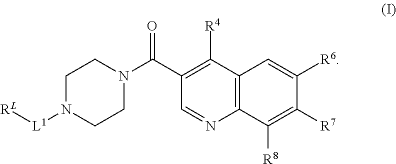

or a pharmaceutically acceptable salt, hydrate, or solvate thereof;
wherein:
   —R⁴ is

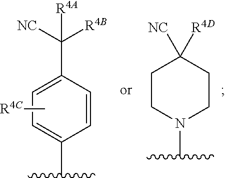

—R^{4A} and —R^{4B} taken together with the carbon atom to which they are attached form a C₃₋₅ cycloalkyl; or —R^{4A} and —R^{4B} are each independently C₁₋₃ alkyl;
—R^{4C} is —H, —F, or —R^{X4C};
—R^{4D} is phenyl, optionally substituted with:
   a) one group —R^{X4D}; and/or
   b) one or more —F;
-L¹- is independently selected from —S(O)₂— and —C(O)—;
—R^{L} is independently selected from C₁₋₆ alkyl and cyclopropyl;
—R⁶ is independently selected from —H, —F, —I, —R^{X6}, and —NO₂;
—R⁷ is independently selected from —H, —F, —I, —R^{X7}, and —NO₂;
—R⁸ is independently selected from —H, —F, —I, —R^{X8}, and —NO₂;
—R^{X4C} is a radioisotope selected from ¹⁸F, ¹²³I, ¹²⁴I, ¹²⁵I, ¹³¹I, and ²¹¹At;
—R^{x4D} is a radioisotope selected from ¹⁸F, ¹²³I, ¹²⁴I, ¹²⁵I, ¹³¹I, and ²¹¹At;
—R^{X6} is a radioisotope selected from ¹⁸F, ¹²³I, ¹²⁴I, ¹²⁵I, ¹³¹I, and ²¹¹At;
—R^{X7} is a radioisotope selected from ¹⁸F, ¹²³I, ¹²⁴I, ¹²⁵I, ¹³¹I, and ²¹¹At; and
—R^{X8} is a radioisotope selected from ¹⁸F, ¹²³I, ¹²⁴I, ¹²⁵I, ¹³¹I, and ²¹¹At;
wherein, the compound comprises only one group selected from —R^{X4C}, —R^{X4D}, —R^{X6}, —R^{X7}, and —R^{X8}; and at least one of —R⁶, —R⁷, and —R⁸ is —H.

(2) The compound according to (1), or a pharmaceutically acceptable salt, hydrate, or solvate thereof, wherein the compound is a radiolabelled compound according to formula (I):

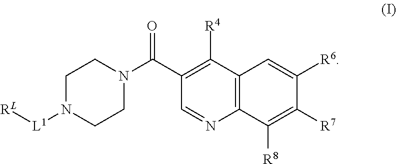

or a pharmaceutically acceptable salt, hydrate, or solvate thereof;
wherein:
   —R⁴ is.

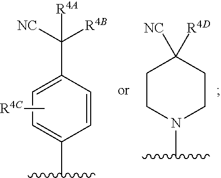

—R^{4A} and —R^{4B} taken together with the carbon atom to which they are attached form a C₃₋₅ cycloalkyl; or —R^{4A} and —R^{4B} are each independently C₁₋₃ alkyl;
—R^{4C} is —H or —F;
—R^{4D} is phenyl, optionally substituted with one of more —F;
-L¹- is independently selected from —S(O)₂— and —C(O)—;
—R^{L} is independently selected from C₁₋₆ alkyl and cyclopropyl;
—R⁶ is independently selected from —H, —F, —I, a radioisotope, and —NO₂;
—R⁷ is independently selected from —H, —F, —I, a radioisotope, and —NO₂;
—R⁸ is independently selected from —H, —F, —I, a radioisotope, and —NO₂;
wherein, one of —R⁶, —R⁷, and —R⁸ is a radioisotope, and the others of —R⁶, —R⁷, and —R⁸ are not radioisotopes; and at least one of —R⁶, —R⁷, and —R⁸ is —H;
wherein the radioisotope is selected from ¹⁸F, ¹²³I, ¹²⁴I, ¹²⁵I, ¹³¹I, and ²¹¹At.

### The Group —R⁴

(3) The compound according to (1) or (2), or a pharmaceutically acceptable salt, hydrate, or solvate thereof, wherein:
—R⁴ is

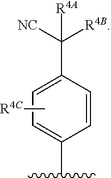

(4) The compound according to any one of (1)-(3), or a pharmaceutically acceptable salt, hydrate, or solvate thereof, wherein:
—R⁴ is

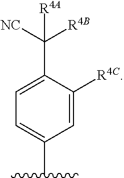

(5) The compound according to (1) or (2), or a pharmaceutically acceptable salt, hydrate, or solvate thereof, wherein:
—R⁴ is

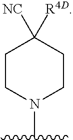

The Groups —R^{4A} and —R^{4B}

(6) The compound according to any one of (1)-(4), or a pharmaceutically acceptable salt, hydrate, or solvate thereof, wherein:
—R^{4A} and —R^{4B}, taken together with the carbon atom to which they are attached form a C₃₋₅ cycloalkyl.

(7) The compound according to any one of (1)-(4), or a pharmaceutically acceptable salt, hydrate, or solvate thereof, wherein:
—R^{4A} and —R^{4B}, taken together with the carbon atom to which they are attached form cyclopropyl.

(8) The compound according to any one of (1)-(4), or a pharmaceutically acceptable salt, hydrate, or solvate thereof, wherein:
—R^{4A} and —R^{4B}, are each independently C₁₋₃ alkyl.

(9) The compound according to any one of (1)-(4), or a pharmaceutically acceptable salt, hydrate, or solvate thereof, wherein:
—R^{4A} and —R^{4B}, are each methyl.

### The Group —R^{4C}

(10) The compound according to any one of (1)-(4) and (6)-(9), as applicable, or a pharmaceutically acceptable salt, hydrate, or solvate thereof, wherein:
—R^{4C} is —H or —R^{X4C}.

(11) The compound according to any one of (1)-(4) and (6)-(9), or a pharmaceutically acceptable salt, hydrate, or solvate thereof, wherein:
—R^{4C} is —H or —F.

(12) The compound according to any one of (1)-(4) and (6)-(9), or a pharmaceutically acceptable salt, hydrate, or solvate thereof, wherein:
—R^{4C} is —H.

(13) The compound according to any one of (1)-(4) and (6)-(9), or a pharmaceutically acceptable salt, hydrate, or solvate thereof, wherein:
—R^{4C} is —F.

(14) The compound according to any one of (1)-(4) and (6)-(9), as applicable, or a pharmaceutically acceptable salt, hydrate, or solvate thereof, wherein:
—R^{4C} is —R^{X4C}.

### The Group —R^{X4C}

(15) The compound according to any one of (1)-(14), or a pharmaceutically acceptable salt, hydrate, or solvate thereof, wherein:
—R^{X4C}, if present, is a radioisotope selected from ¹⁸F, ¹²³I, ¹²⁴I, ¹²⁵I, and ¹³¹I.

(16) The compound according to any one of (1)-(14), or a pharmaceutically acceptable salt, hydrate, or solvate thereof, wherein:
—R^{X4C}, if present, is a radioisotope that is ¹⁸F.

(17) The compound according to any one of (1)-(14), or a pharmaceutically acceptable salt, hydrate, or solvate thereof, wherein:
—R^{X4C}, if present, is a radioisotope selected from ¹²³I, ¹²⁴I, ¹²⁵I, and ¹³¹I.

(18) The compound according to any one of (1)-(14), or a pharmaceutically acceptable salt, hydrate, or solvate thereof, wherein:
—R^{X4C}, if present, is a radioisotope selected from ¹²⁵I and ¹³¹I.

(19) The compound according to any one of (1)-(14), or a pharmaceutically acceptable salt, hydrate, or solvate thereof, wherein:
—R^{X4C}, if present, is a radioisotope that is ¹²⁵I.

### The Group —R^{4D}

(20) The compound according to any one of (1), (2) and (5), or a pharmaceutically acceptable salt, hydrate, or solvate thereof, wherein:
—R^{4D} is phenyl optionally substituted with one or more —F.

(21) The compound according to any one of (1), (2) and (5), or a pharmaceutically acceptable salt, hydrate, or solvate thereof, wherein:
—R^{4D} is phenyl.

(22) The compound according to any one of (1), (2) and (5), or a pharmaceutically acceptable salt, hydrate, or solvate thereof, wherein:
—R^{4D} is phenyl substituted with —F.

(23) The compound according to (1) or (5), as applicable, or a pharmaceutically acceptable salt, hydrate, or solvate thereof, wherein:
—R^{4D} is phenyl optionally substituted with one —R^{X4D}.

(24) The compound according to (1) or (5), as applicable, or a pharmaceutically acceptable salt, hydrate, or solvate thereof, wherein:
—R^{4D} is phenyl substituted with one —R^{X4D}.

(25) The compound according to (1) or (5), as applicable, or a pharmaceutically acceptable salt, hydrate, or solvate thereof, wherein:
—R^{4D} is

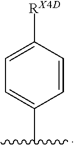

### The Group —R^{X4D}

(26) The compound according to any one of (1)-(25), or a pharmaceutically acceptable salt, hydrate, acceptable salt, hydrate, or solvate thereof, wherein:
—R^{X4D}, if present, is a radioisotope selected from ¹⁸F, ¹²³I, ¹²⁴I, ¹²⁵I, and ¹³¹I.

(27) The compound according to any one of (1)-(25), or a pharmaceutically acceptable salt, hydrate, or solvate thereof, wherein:
—R^{X4D}, if present, is a radioisotope that is ¹⁸F.

(28) The compound according to any one of (1)-(25), or a pharmaceutically acceptable salt, salt, hydrate, or solvate thereof, wherein:
—R^{X4D}, if present, is a radioisotope selected from ¹²³I, ¹²⁴I, ¹²⁵I, and ¹³¹I.

(29) The compound according to any one of (1)-(25), or a pharmaceutically acceptable salt, hydrate, or solvate thereof, wherein:
—R^{X4D}, if present, is a radioisotope selected from ¹²⁵I and ¹³¹I.

(30) The compound according to any one of (1)-(25), or a pharmaceutically acceptable salt, hydrate, or solvate thereof, wherein:
—R^{X4D}, if present, is a radioisotope that is ¹²⁵I.

### The Group -L¹-

(31) The compound according to any one of (1)-(30), or a pharmaceutically acceptable salt, hydrate, or solvate thereof, wherein:
-L¹- is —S(O)₂—.

(32) The compound according to any one of (1)-(30), or a pharmaceutically acceptable salt, hydrate, or solvate thereof, wherein:
-L¹- is —C(O)—.

### The Group —R^{L}

(33) The compound according to any one of (1)-(32), or a pharmaceutically acceptable salt, hydrate, or solvate thereof, wherein:
—R^{L}— is C₁₋₆ alkyl.

(34) The compound according to any one of (1)-(32), or a pharmaceutically acceptable salt, hydrate, or solvate thereof, wherein:
—R^{L}— is C₁₋₃ alkyl.

(35) The compound according to any one of (1)-(32), or a pharmaceutically acceptable salt, hydrate, or solvate thereof, wherein:
—R^{L}— is methyl.

(36) The compound according to any one of (1)-(32), or a pharmaceutically acceptable salt, hydrate, or solvate thereof, wherein:
—R^{L}— is cyclopropyl.

### The Radioisotope

Where the atomic mass of a given atom is defined, e.g., in the case of a radioisotope, then the structural formula does not contemplate isotopic replacements at that position. For example, when a position is defined as being ¹⁸F, then it is not contemplated that that fluorine atom may be replaced with other isotopic forms such as ¹⁹F.

In this context, as used herein, when a substituent position is defined as being “a radioisotope”, then that means that the substituent at that position is enriched in a radioisotope, for example, ¹⁸F, ¹²³I, ¹²⁴I, ¹²⁵I, ¹³¹I, or ²¹¹At, such that an amount of the compound comprises the radioisotope at an abundance that is higher than the natural abundance of said radioisotope.

As noted hereinabove, the PQM-H compounds are enriched with a radioisotope at one substituent position, i.e., the PQM-H compounds described herein are enriched with a radioisotope at one, and no more than one substituent position. More specifically, a PQM-H compound comprises only one group selected from —R^{X4C}, —R^{X4D}, —R^{X6}, —R^{X7}, and —R^{X8} (each of which is defined as a radioisotope). That is, in the PQM-H compounds described herein one of —R^{X4C}, —R^{X4D}, —R^{X6}, —R^{X7}, and —R^{X8} is present and the others of —R^{X4C}, —R^{X4D}, —R^{X6}, —R^{X7}, and —R^{X8} are not present. In other words, the compound comprises a radioisotope at the position of one of —R^{X4C}, —R^{X4D}, —R⁶, —R⁷, and —R⁸.

(37) The compound according to any one of (1)-(36), or a pharmaceutically acceptable salt, hydrate, or solvate thereof, wherein:
the radioisotope is selected from ¹⁸F, ¹²³I, ¹²⁴I, ¹²⁵I, and ¹³¹I.

(38) The compound according to any one of (1)-(36), or a pharmaceutically acceptable salt, hydrate, or solvate thereof, wherein:
the radioisotope is ¹⁸F.

(39) The compound according to any one of (1)-(36), or a pharmaceutically acceptable salt, hydrate, or solvate thereof, wherein:
the radioisotope is selected from ¹²³I, ¹²⁴I, ¹²⁵I, and ¹³¹I.

(40) The compound according to any one of (1)-(36), or a pharmaceutically acceptable salt, hydrate, or solvate thereof, wherein:
the radioisotope is selected from ¹²⁵I and ¹³¹I.

(41) The compound according to any one of (1)-(36), or a pharmaceutically acceptable salt, hydrate, or solvate thereof, wherein:
the radioisotope is ¹²⁵I.

(42) The compound according to any one of (1)-(36), or a pharmaceutically acceptable salt, hydrate, or solvate thereof, wherein:
the radioisotope is ¹³¹I.

(43) The compound according to any one of (1)-(36), or a pharmaceutically acceptable salt, hydrate, or solvate thereof, wherein:
the radioisotope is ¹²⁴I.

(44) The compound according to any one of (1)-(36), or a pharmaceutically acceptable salt, hydrate, or solvate thereof, wherein:
the radioisotope is ¹²³I.

(45) The compound according to any one of (1)-(36), or a pharmaceutically acceptable salt, hydrate, or solvate thereof, wherein:
the radioisotope is ²¹¹At.

(46) The compound according to any one of (1)-(45), as applicable, or a pharmaceutically acceptable salt, hydrate, or solvate thereof, wherein:
one of —R⁶, —R⁷, or —R⁸ is the radioisotope, for example —R⁶ is —R^{X6}, —R⁷ is —R^{X7}, or —R⁸ is —R^{X8}.

(47) The compound according to any one of (1)-(45), as applicable, or a pharmaceutically acceptable salt, hydrate, or solvate thereof, wherein:
—R⁶ or —R⁷ is the radioisotope, for example —R⁶ is —R^{X6}, or —R⁷ is —R^{X7}.

(48) The compound according to any one of (1)-(45), as applicable, or a pharmaceutically acceptable salt, hydrate, or solvate thereof, wherein:
—R⁶ or —R⁸ is the radioisotope, for example —R⁶ is —R^{X6}, or —R⁸ is —R^{X8}.

### The Group —R⁶

(49) The compound according to any one of (1)-(48), as applicable, or a pharmaceutically acceptable salt, hydrate, or solvate thereof, wherein:
—R⁶ is selected from —H, —F, —I, a radioisotope, and —NO₂.

(50) The compound according to any one of (1)-(48), as applicable, or a pharmaceutically acceptable salt, hydrate, or solvate thereof, wherein:
—R⁶ is selected from —H, —R^{X6}, and —NO₂.

(51) The compound according to any one of (1)-(48), as applicable, or a pharmaceutically acceptable salt, hydrate, or solvate thereof, wherein:
—R⁶ is selected from —H and —R^{X6}.

(52) The compound according to any one of (1)-(48), or a pharmaceutically acceptable salt, hydrate, or solvate thereof, wherein:
—R⁶ is a not a radioisotope and is selected from —H, —F, —I, and —NO₂.

(53) The compound according to any one of (1)-(48), or a pharmaceutically acceptable salt, hydrate, or solvate thereof, wherein:
—R⁶ is a not a radioisotope and is selected from —H, and —NO₂.

(54) The compound according to any one of (1)-(48), or a pharmaceutically acceptable salt, hydrate, or solvate thereof, wherein:
—R⁶ is a not a radioisotope and is selected from —H, —F, and —I.

(55) The compound according to any one of (1)-(48), or a pharmaceutically acceptable salt, hydrate, or solvate thereof, wherein:
—R⁶ is a not a radioisotope and is selected from —H, and —F.

(56) The compound according to any one of (1)-(48), or a pharmaceutically acceptable salt, hydrate, or solvate thereof, wherein:
—R⁶ is a not a radioisotope and is —H.

(57) The compound according to any one of (1)-(48), as applicable, or a pharmaceutically acceptable salt, hydrate, or solvate thereof, wherein:
—R⁶ is the radioisotope, for example, —R⁶ is —R^{X6}.

### The Group —R⁷

(58) The compound according to any one of (1)-(57), as applicable, or a pharmaceutically acceptable salt, hydrate, or solvate thereof, wherein:
—R⁷ is selected from —H, —F, —I, a radioisotope, and —NO₂.

(59) The compound according to any one of (1)-(57), as applicable, or a pharmaceutically acceptable salt, hydrate, or solvate thereof, wherein:
—R⁷ is selected from —H, —R^{X7}, and —NO₂.

(60) The compound according to any one of (1)-(57), as applicable, or a pharmaceutically acceptable salt, hydrate, or solvate thereof, wherein:
—R⁷ is selected from —H and —R^{X7}.

(61) The compound according to any one of (1)-(57), or a pharmaceutically acceptable salt, hydrate, or solvate thereof, wherein:
—R⁷ is a not a radioisotope and is selected from —H, —F, —I, and —NO₂.

(62) The compound according to any one of (1)-(57), or a pharmaceutically acceptable salt, hydrate, or solvate thereof, wherein:
—R⁷ is a not a radioisotope and is selected from —H, and —NO₂.

(63) The compound according to any one of (1)-(57), or a pharmaceutically acceptable salt, hydrate, or solvate thereof, wherein:
—R⁷ is a not a radioisotope and is selected from —H, —F, and —I.

(64) The compound according to any one of (1)-(57), or a pharmaceutically acceptable salt, hydrate, or solvate thereof, wherein:
—R⁷ is a not a radioisotope and is selected from —H, and —F.

(65) The compound according to any one of (1)-(57), or a pharmaceutically acceptable salt, hydrate, or solvate thereof, wherein:
—R⁷ is a not a radioisotope and is —H.

(66) The compound according to any one of (1)-(57), as applicable, or a pharmaceutically acceptable salt, hydrate, or solvate thereof, wherein:
—R⁷ is the radioisotope, for example, —R⁷ is —R^{X7}.

### The Group —R⁸

(67) The compound according to any one of (1)-(66), as applicable, or a pharmaceutically acceptable salt, hydrate, or solvate thereof, wherein:
—R⁸ is selected from —H, —F, —I, a radioisotope, and —NO₂.

(68) The compound according to any one of (1)-(66), as applicable, or a pharmaceutically acceptable salt, hydrate, or solvate thereof, wherein:
—R⁸ is selected from —H, —R^{X8}, and —NO₂.

(69) The compound according to any one of (1)-(66), as applicable, or a pharmaceutically acceptable salt, hydrate, or solvate thereof, wherein:
—R⁸ is selected from —H and —R^{X8}.

(70) The compound according to any one of (1)-(66), or a pharmaceutically acceptable salt, hydrate, or solvate thereof, wherein:
—R⁸ is a not a radioisotope and is selected from —H, —F, —I, and —NO₂.

(71) The compound according to any one of (1)-(66), or a pharmaceutically acceptable salt, hydrate, or solvate thereof, wherein:
—R⁸ is a not a radioisotope and is selected from —H, and —NO₂.

(72) The compound according to any one of (1)-(66), or a pharmaceutically acceptable salt, hydrate, or solvate thereof, wherein:
—R⁸ is a not a radioisotope and is selected from —H, —F, and —I.

(73) The compound according to any one of (1)-(66), or a pharmaceutically acceptable salt, hydrate, or solvate thereof, wherein:
—R⁸ is a not a radioisotope and is selected from —H, and —F.

(74) The compound according to any one of (1)-(66), or a pharmaceutically acceptable salt, hydrate, or solvate thereof, wherein:
—R⁸ is a not a radioisotope and is —H.

(75) The compound according to any one of (1)-(66), as applicable, or a pharmaceutically acceptable salt, hydrate, or solvate thereof, wherein:
—R⁸ is the radioisotope, for example, —R⁸ is —R^{X8}.

### The Group —Rx⁶

(76) The compound according to any one of (1)-(77), or a pharmaceutically acceptable salt, hydrate, or solvate thereof, wherein:
—R^{X6}, if present, is a radioisotope selected from ¹⁸F, ¹²³I, ¹²⁴I, ¹²⁵I, and ¹³¹I.

(77) The compound according to any one of (1)-(77), or a pharmaceutically acceptable salt, hydrate, or solvate thereof, wherein:
—R^{X6}, if present, is a radioisotope that is ¹⁸F.

(78) The compound according to any one of (1)-(77), or a pharmaceutically acceptable salt, hydrate, or solvate thereof, wherein:
—R^{X6}, if present, is a radioisotope selected from ¹²³I, ¹²⁴I, ¹²⁵I, and ¹³¹I.

(79) The compound according to any one of (1)-(77), or a pharmaceutically acceptable salt, hydrate, or solvate thereof, wherein:
—R^{X6}, if present, is a radioisotope selected from ¹²⁵I and ¹³¹I.

(80) The compound according to any one of (1)-(77), or a pharmaceutically acceptable salt, hydrate, or solvate thereof, wherein:
—R^{X6}, if present, is a radioisotope that is ¹²⁵I.

### The Group —R^{X7}

(81) The compound according to any one of (1)-(80), or a pharmaceutically acceptable salt, hydrate, or solvate thereof, wherein:
—R^{X7}, if present, is a radioisotope selected from ¹⁸F, ¹²³I, ¹²⁴I, ¹²⁵I, and ¹³¹I.

(82) The compound according to any one of (1)-(80), or a pharmaceutically acceptable salt, hydrate, or solvate thereof, wherein:
—R^{X7}, if present, is a radioisotope that is ¹⁸F.

(83) The compound according to any one of (1)-(80), or a pharmaceutically acceptable salt, hydrate, or solvate thereof, wherein:
—R^{X7}, if present, is a radioisotope selected from ¹²³I, ¹²⁴I, ¹²⁵I, and ¹³¹I.

(84) The compound according to any one of (1)-(80), or a pharmaceutically acceptable salt, hydrate, or solvate thereof, wherein:
—R^{X7}, if present, is a radioisotope selected from ¹²⁵I and ¹³¹I.

(85) The compound according to any one of (1)-(80), or a pharmaceutically acceptable salt, hydrate, or solvate thereof, wherein:
—R^{X7}, if present, is a radioisotope that is ¹²⁵I.

### The Group —R^{X8}

(86) The compound according to any one of (1)-(85), or a pharmaceutically acceptable salt, hydrate, or solvate thereof, wherein:
—R^{X8}, if present, is a radioisotope selected from ¹⁸F, ¹²³I, ¹²⁴I, ¹²⁵I, and ¹³¹I.

(87) The compound according to any one of (1)-(85), or a pharmaceutically acceptable salt, hydrate, or solvate thereof, wherein:
—R^{X8}, if present, is a radioisotope that is ¹⁸F.

(88) The compound according to any one of (1)-(85), or a pharmaceutically acceptable salt, hydrate, or solvate thereof, wherein:
—R^{X8}, if present, is a radioisotope selected from ¹²³I, ¹²⁴I, ¹²⁵I, and ¹³¹I.

(89) The compound according to any one of (1)-(85), or a pharmaceutically acceptable salt, hydrate, or solvate thereof, wherein:
—R^{X8}, if present, is a radioisotope selected from ¹²⁵I and ¹³¹I.

(90) The compound according to any one of (1)-(85), or a pharmaceutically acceptable salt, hydrate, or solvate thereof, wherein:
—R^{X8}, if present, is a radioisotope that is ¹²⁵I.

### Some Preferred Combinations

(91) The compound according to any one of (1)-(90), or a pharmaceutically acceptable salt or solvate thereof, wherein:
—R^{X4C}, if present, is a radioisotope that is ¹⁸F;
—R^{X4D}, if present, is a radioisotope that is ¹⁸F;
—R^{X6}, if present, is a radioisotope selected from ¹⁸F, ¹²³I, ¹²⁴I, ¹²⁵I, and ¹³¹I;
—R^{X7}, if present, is a radioisotope selected from ¹⁸F, ¹²³I, ¹²⁴I, ¹²⁵I, and ¹³¹I; and
—R^{X8}, if present, is a radioisotope selected from ¹⁸F, ¹²³I, ¹²⁴I, ¹²⁵I, and ¹³¹I.

(92) The compound according to any one of (1)-(36), or a pharmaceutically acceptable salt or solvate thereof, wherein:
—R⁶ is the radioisotope, for example, —R⁶ is —R^{X6};
—R⁷ is —H; and —R⁸ is —H or —NO₂.

(93) The compound according to any one of (1)-(36), or a pharmaceutically acceptable salt, hydrate, or solvate thereof, wherein:
—R⁶ is the radioisotope, for example, —R⁶ is —R^{X6};
—R⁷ is —H; —R⁸ is —H or —NO₂; and the radioisotope is ¹⁸F.

(94) The compound according to any one of (1)-(36), or a pharmaceutically acceptable salt, hydrate, or solvate thereof, wherein:
—R⁶ is the radioisotope, for example, —R⁶ is —R^{X6};
—R⁷ is —H; —R⁸ is —H or —NO₂; and the radioisotope is selected from ¹²³I, ¹²⁴I, ¹²⁵I, ¹³¹I and ²¹¹At.

(95) The compound according to any one of (1)-(36), or a pharmaceutically acceptable salt, hydrate, or solvate thereof, wherein:
—R⁶ is the radioisotope, for example, —R⁶ is —R^{X6};
—R⁷ is —H; —R⁸ is —H or —NO₂; and the radioisotope is selected from ¹²³I, ¹²⁴I, ¹²⁵I, and ¹³¹I.

(96) The compound according to any one of (1)-(36), or a pharmaceutically acceptable salt, hydrate, or solvate thereof, wherein:
—R⁶ is the radioisotope, for example, —R⁶ is —R^{X6};
—R⁷ is —H; —R⁸ is —H or —NO₂; and the radioisotope is ¹²⁵I.

(97) The compound according to any one of (1)-(36), or a pharmaceutically acceptable salt, hydrate, or solvate thereof, wherein:
—R⁷ is the radioisotope, for example, —R⁷ is —R^{X7}; and
—R⁶ is —H; —R⁸ is —H.

(98) The compound according to any one of (1)-(36), or a pharmaceutically acceptable salt, hydrate, or solvate thereof, wherein:
—R⁷ is the radioisotope, for example, —R⁷ is —R^{X7};
—R⁶ is —H; —R⁸ is —H; and
the radioisotope is ¹⁸F.

(99) The compound according to any one of (1)-(36), or a pharmaceutically acceptable salt, hydrate, or solvate thereof, wherein:
—R⁷ is the radioisotope, for example, —R⁷ is —R^{X7};
—R⁶ is —H; —R⁸ is —H; and the radioisotope is selected from ¹²³I, ¹²⁴I, ¹²⁵I, ¹³¹I and ²¹¹At.

(100) The compound according to any one of (1)-(36), or a pharmaceutically acceptable salt, hydrate, or solvate thereof, wherein:
—R⁷ is the radioisotope, for example, —R⁷ is —R^{X7};
—R⁶ is —H; —R⁸ is —H; and
the radioisotope is selected from ¹²³I, ¹²⁴I, ¹²⁵I, and ¹³¹I.

(101) The compound according to any one of (1)-(36), or a pharmaceutically acceptable salt, hydrate, or solvate thereof, wherein:
—R⁷ is the radioisotope, for example, —R⁷ is —R^{X7};
—R⁶ is —H; —R⁸ is —H; and
the radioisotope is ¹²⁵I.

(102) The compound according to any one of (1)-(36), or a pharmaceutically acceptable salt, hydrate, or solvate thereof, wherein:
—R⁶ is —H or —NO₂; —R⁷ is —H; and
—R⁸ is the radioisotope, for example, —R⁸ is —R^{X8}.

(103) The compound according to any one of (1)-(36), or a pharmaceutically acceptable salt, hydrate, or solvate thereof, wherein:
—R⁶ is —H or —NO₂; —R⁷ is —H;
—R⁸ is the radioisotope, for example, —R⁸ is —R^{X8}; and the radioisotope is ¹⁸F.

(104) The compound according to any one of (1)-(36), or a pharmaceutically acceptable salt, hydrate, or solvate thereof, wherein:
—R⁶ is —H or —NO₂; —R⁷ is —H;
—R⁸ is the radioisotope, for example, —R⁸ is —R^{X8}; and
the radioisotope is selected from ¹²³I, ¹²⁴I, ¹²⁵I, ¹³¹I and ²¹¹At.

(105) The compound according to any one of (1)-(36), or a pharmaceutically acceptable salt, hydrate, or solvate thereof, wherein:
—R⁶ is —H or —NO₂; —R⁷ is —H;
—R⁸ is the radioisotope, for example, —R⁸ is —R^{X8}; and
the radioisotope is selected from ¹²³I, ¹²⁴I, ¹²⁵I, and ¹³¹I.

(106) The compound according to any one of (1)-(36), or a pharmaceutically acceptable salt, hydrate, or solvate thereof, wherein:
—R⁶ is —H or —NO₂; —R⁷ is —H;
—R⁸ is the radioisotope, for example, —R⁸ is —R^{X8}; and
the radioisotope is ¹²⁵I.

(107) The compound according to any one of (1)-(36), as applicable, or a pharmaceutically acceptable salt, hydrate, or solvate thereof, wherein:
—R^{4C}, if present, is —H, —F, or —R^{X4C};
—R^{4D}, if present, is phenyl optionally substituted with one group —R^{X4D};
—R⁶ is independently selected from —H, —F, —I, and —NO₂;
—R⁷ is independently selected from —H, —F, —I, and —NO₂; and
—R⁸ is independently selected from —H, —F, —I, and —NO₂.

(108) The compound according to any one of (1)-(36), as applicable, or a pharmaceutically acceptable salt, hydrate, or solvate thereof, wherein:
—R^{4C}, if present, is —H, —F, or —R^{X4C};
—R^{4D}, if present, is phenyl optionally substituted with one group —R^{X4D};
—R⁶ is independently selected from —H, —F, —I, and —NO₂;
—R⁷ is independently selected from —H, —F, —I, and —NO₂;
—R⁸ is independently selected from —H, —F, —I, and —NO₂;
—R^{X4C} is a radioisotope selected from ¹⁸F, ¹²³I, ¹²⁴I, ¹²⁵I, and ¹³¹I; and
—R^{X4D} is a radioisotope selected from ¹⁸F, ¹²³I, ¹²⁴I, ¹²⁵I, and ¹³¹I.

(109) The compound according to any one of (1)-(36), as applicable, or a pharmaceutically acceptable salt, hydrate, or solvate thereof, wherein:
—R^{4C}, if present, is —H, —F, or —R^{X4C};
—R^{4D}, if present, is phenyl optionally substituted with one group —R^{X4D};
—R⁶ is independently selected from —H, —F, —I, and —NO₂;
—R⁷ is independently selected from —H, —F, —I, and —NO₂;
—R⁸ is independently selected from —H, —F, —I, and —NO₂;
—R^{X4C} is a radioisotope that is ¹⁸F; and
—R^{X4D} is a radioisotope that is ¹⁸F.

(110) The compound according to (1), or a pharmaceutically acceptable salt, hydrate, or solvate thereof, wherein:
—R⁴ is

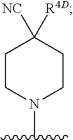

and
—R^{4D} is phenyl substituted with one group —R^{X4D}.

(111) The compound according to (1), or a pharmaceutically acceptable salt, hydrate, or solvate thereof, wherein:
—R⁴ is

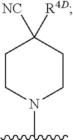

—R^{4D} is phenyl substituted with one group —R^{X4D};
—R⁶ is —H; —R⁷ is —H; and —R⁸ is —H.

(112) The compound according to (1), or a pharmaceutically acceptable salt, hydrate, or solvate thereof, wherein:
—R⁴ is

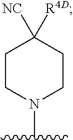

—R^{4D} is phenyl substituted with one group —R^{X4D};
-L¹- is —S(O)₂—; —R^{L} is selected from C₁₋₈ alkyl; and
—R⁶ is —H; —R⁷ is —H; and —R⁸ is —H.

(113) The compound according to (1), or a pharmaceutically acceptable salt, hydrate, or solvate thereof, wherein:
—R⁴ is

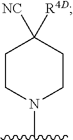

—R^{4D} is phenyl substituted with one group —R^{X4D};
-L¹- is —S(O)₂—; —R^{L} is selected from C₁₋₈ alkyl; and
—R⁶ is —H; —R⁷ is —H; —R⁸ is —H; and
—R^{X4D} is selected from ¹⁸F, ¹²³I, ¹²⁴I, ¹²⁵I, and ¹³¹I.

(114) The compound according to (1), or a pharmaceutically acceptable salt, hydrate, or solvate thereof, wherein:
—R⁴ is

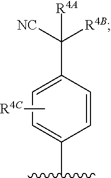

and
—R⁶ is the radioisotope, for example, —R⁶ is —R^{X6}.

(115) The compound according to (1), or a pharmaceutically acceptable salt, hydrate, or solvate thereof, wherein:
—R⁴ is

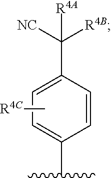

—R^{4A} and —R^{4B} together with the carbon atom to which they are attached form a C₃₋₅ cycloalkyl;
—R^{4C} is —H; and
—R⁶ is the radioisotope, for example, —R⁶ is —R^{X6}.

(116) The compound according to (1), or a pharmaceutically acceptable salt, hydrate, or solvate thereof, wherein:
—R⁴ is

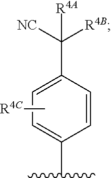

—R^{4A} and —R^{4B} together with the carbon atom to which they are attached form a C₃₋₅ cycloalkyl;
—R^{4C} is —H;
-L¹- is —S(O)₂—; —R^{L} is selected from C₁₋₆ alkyl; and
—R⁶ is the radioisotope, for example, —R⁶ is —R^{X6}.

(117) The compound according to (1), or a pharmaceutically acceptable salt, hydrate, or solvate thereof, wherein:
—R⁴ is

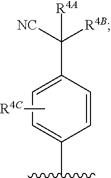

—R^{4A} and —R^{4B} together with the carbon atom to which they are attached form a C₃₋₅ cycloalkyl;
—R^{4C} is —H;
-L¹- is —S(O)₂—; —R^{L} is selected from C₁₋₆ alkyl;
—R⁷ is —H; —R⁸ is —H; and —R⁶ is the radioisotope, for example, —R⁶ is —R^{X6}.

(118) The compound according to (1), or a pharmaceutically acceptable salt, hydrate, or solvate thereof, wherein:
—R⁴ is

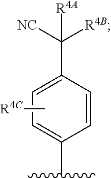

—R^{4A} and —R^{4B} together with the carbon atom to which they are attached form a C₃₋₅ cycloalkyl;
—R^{4C} is —H;
-L¹- is —S(O)₂—; —R^{L} is selected from C₁₋₆ alkyl;
—R⁷ is —H; —R⁸ is —H; —R⁶ is the radioisotope, for example, —R⁶ is —R^{X6}; and the radioisotope is selected from ¹⁸F, ¹²³I, ¹²⁴I, ¹²⁵I, and ¹³¹I.

(119) The compound according to (1), or a pharmaceutically acceptable salt, hydrate, or solvate thereof, wherein:
—R⁴ is

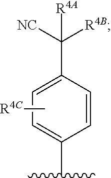

—R^{4A} and —R^{4B} together with the carbon atom to which they are attached form a C₃₋₅ cycloalkyl;
—R^{4C} is —H;
-L¹- is —S(O)₂—; —R^{L} is selected from C₁₋₆ alkyl;
—R⁷ is —H; —R⁸ is —H; —R⁶ is the radioisotope, for example, —R⁶ is —R^{X6}; and the radioisotope is selected from ¹⁸F, and ¹²⁵I.

### Specific Compounds

(120) A compound according to (1), selected from compounds of the following formulae and pharmaceutically acceptable salts, hydrates, and solvates thereof:

| Cmpd No. | Structure | Name |
|---|---|---|
| PQM-H-001 | 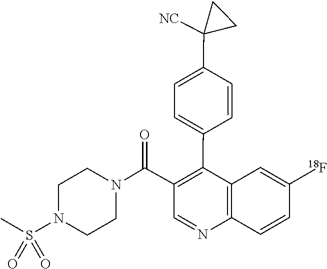 [SMILES: CS(=O)(=O)N1CCN(C(=O)c2cnc3ccc([18F])cc3c2-c2ccc(C3(C#N)CC3)cc2)CC1] | 1-(4-(6-(fluoro-¹⁸F)-3-(4- (methylsulfonyl)piperazine-1- carbonyl)quinolin-4- yl)phenyl)cyclopropane-1- carbonitrile |
| PQM-H-002 | 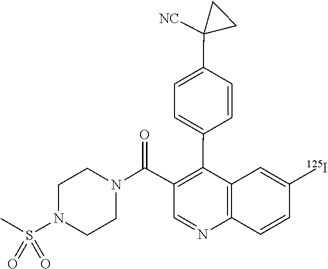 [SMILES: CS(=O)(=O)N1CCN(C(=O)c2cnc3ccc([125I])cc3c2-c2ccc(C3(C#N)CC3)cc2)CC1] | 1-(4-(6-(iodo-¹²⁵I)-3-(4- (methylsulfonyl)piperazine-1- carbonyl)quinolin-4- yl)phenyl)cyclopropane-1- carbonitrile |
| PQM-H-003 | 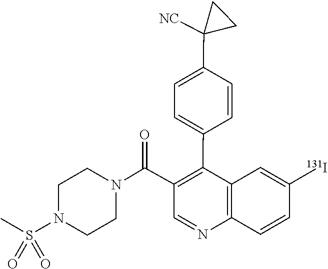 [SMILES: CS(=O)(=O)N1CCN(C(=O)c2cnc3ccc([131I])cc3c2-c2ccc(C3(C#N)CC3)cc2)CC1] | 1-(4-(6-(iodo-¹³¹I)-3-(4- (methylsulfonyl)piperazine-1- carbonyl)quinolin-4- yl)phenyl)cyclopropane-1- carbonitrile |
| PQM-H-004 | 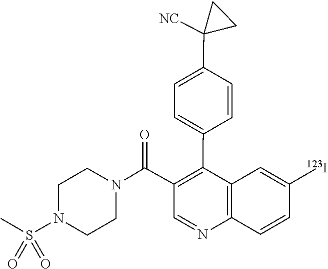 [SMILES: CS(=O)(=O)N1CCN(C(=O)c2cnc3ccc([123I])cc3c2-c2ccc(C3(C#N)CC3)cc2)CC1] | 1-(4-(6-(iodo-¹²³I)-3-(4- (methylsulfonyl)piperazine-1- carbonyl)quinolin-4- yl)phenyl)cyclopropane-1- carbonitrile |
| PQM-H-005 | 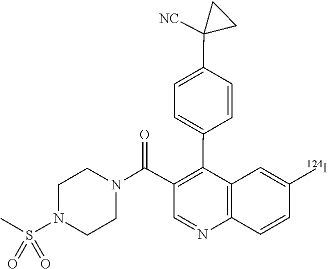 [SMILES: CS(=O)(=O)N1CCN(C(=O)c2cnc3ccc([124I])cc3c2-c2ccc(C3(C#N)CC3)cc2)CC1] | 1-(4-(6-(iodo-¹²⁴I)-3-(4- (methylsulfonyl)piperazine-1- carbonyl)quinolin-4- yl)phenyl)cyclopropane-1- carbonitrile |
| PQM-H-006 | 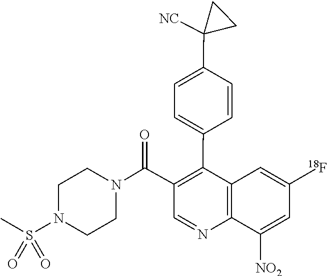 [SMILES: CS(=O)(=O)N1CCN(C(=O)c2cnc3c([N+](=O)[O-])cc([18F])cc3c2-c2ccc(C3(C#N)CC3)cc2)CC1] | 1-(4-(6-(fluoro-¹⁸F)-3-(4- (methylsulfonyl)piperazine-1- carbonyl)-8-nitroquinolin-4- yl)phenyl)cyclopropane-1- carbonitrile |
| PQM-H-007 | 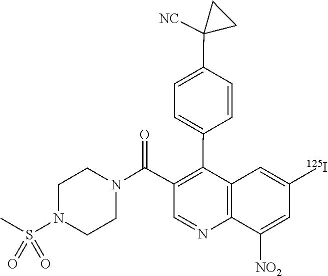 [SMILES: CS(=O)(=O)N1CCN(C(=O)c2cnc3c([N+](=O)[O-])cc([125I])cc3c2-c2ccc(C3(C#N)CC3)cc2)CC1] | 1-(4-(6-(iodo-¹²⁵I)-3-(4- (methylsulfonyl)piperazine-1- carbonyl)-8-nitroquinolin-4- yl)phenyl)cyclopropane-1- carbonitrile |
| PQM-H-008 | 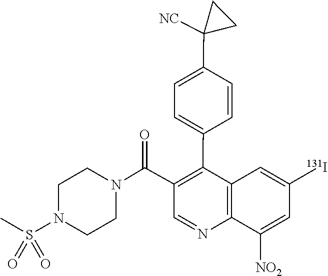 [SMILES: CS(=O)(=O)N1CCN(C(=O)c2cnc3c([N+](=O)[O-])cc([131I])cc3c2-c2ccc(C3(C#N)CC3)cc2)CC1] | 1-(4-(6-(iodo-¹³¹I)-3-(4- (methylsulfonyl)piperazine-1- carbonyl)-8-nitroquinolin-4- yl)phenyl)cyclopropane-1- carbonitrile |
| PQM-H-009 | 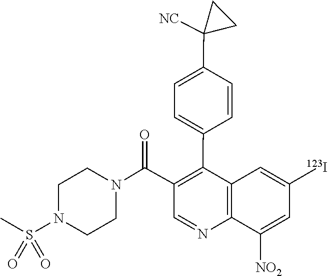 [SMILES: CS(=O)(=O)N1CCN(C(=O)c2cnc3c([N+](=O)[O-])cc([123I])cc3c2-c2ccc(C3(C#N)CC3)cc2)CC1] | 1-(4-(6-(iodo-¹²³I)-3-(4- (methylsulfonyl)piperazine-1- carbonyl)-8-nitroquinolin-4- yl)phenyl)cyclopropane-1- carbonitrile |
| PQM-H-010 | 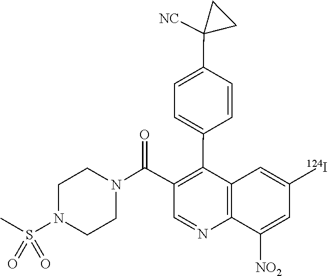 [SMILES: CS(=O)(=O)N1CCN(C(=O)c2cnc3c([N+](=O)[O-])cc([124I])cc3c2-c2ccc(C3(C#N)CC3)cc2)CC1] | 1-(4-(6-(iodo-¹²⁴I)-3-(4- (methylsulfonyl)piperazine-1- carbonyl)-8-nitroquinolin-4- yl)phenyl)cyclopropane-1- carbonitrile |
| PQM-H-011 | 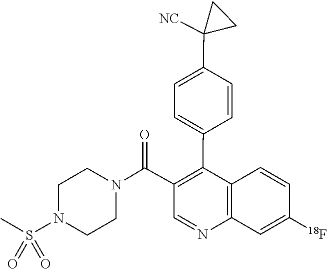 [SMILES: CS(=O)(=O)N1CCN(C(=O)c2cnc3cc([18F])ccc3c2-c2ccc(C3(C#N)CC3)cc2)CC1] | 1-(4-(7-(fluoro-¹⁸F)-3-(4- (methylsulfonyl)piperazine-1- carbonyl)quinolin-4- yl)phenyl)cyclopropane-1- carbonitrile |
| PQM-H-012 | 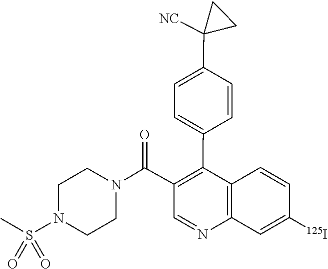 [SMILES: CS(=O)(=O)N1CCN(C(=O)c2cnc3cc([125I])ccc3c2-c2ccc(C3(C#N)CC3)cc2)CC1] | 1-(4-(7-(iodo-¹²⁵I)-3-(4- (methylsulfonyl)piperazine-1- carbonyl)quinolin-4- yl)phenyl)cyclopropane-1- carbonitrile |
| PQM-H-013 | 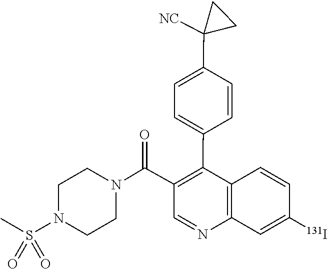 [SMILES: CS(=O)(=O)N1CCN(C(=O)c2cnc3cc([131I])ccc3c2-c2ccc(C3(C#N)CC3)cc2)CC1] | 1-(4-(7-(iodo-¹³¹I)-3-(4- (methylsulfonyl)piperazine-1- carbonyl)quinolin-4- yl)phenyl)cyclopropane-1- carbonitrile |
| PQM-H-014 | 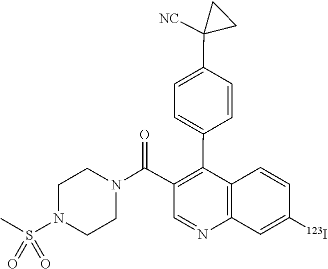 [SMILES: CS(=O)(=O)N1CCN(C(=O)c2cnc3cc([123I])ccc3c2-c2ccc(C3(C#N)CC3)cc2)CC1] | 1-(4-(7-(iodo-¹²³I)-3-(4- (methylsulfonyl)piperazine-1- carbonyl)quinolin-4- yl)phenyl)cyclopropane-1- carbonitrile |
| PQM-H-015 | 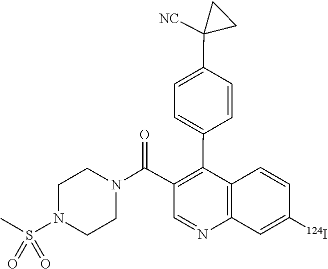 [SMILES: CS(=O)(=O)N1CCN(C(=O)c2cnc3cc([124I])ccc3c2-c2ccc(C3(C#N)CC3)cc2)CC1] | 1-(4-(7-(iodo-¹²⁴I)-3-(4- (methylsulfonyl)piperazine-1- carbonyl)quinolin-4- yl)phenyl)cyclopropane-1- carbonitrile |
| PQM-H-016 | 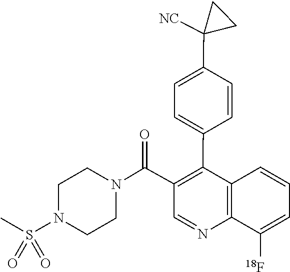 [SMILES: CS(=O)(=O)N1CCN(C(=O)c2cnc3c([18F])cccc3c2-c2ccc(C3(C#N)CC3)cc2)CC1] | 1-(4-(8-(fluoro-¹⁸F)-3-(4- (methylsulfonyl)piperazine-1- carbonyl)quinolin-4- yl)phenyl)cyclopropane-1- carbonitrile |
| PQM-H-017 | 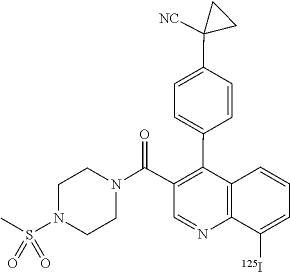 [SMILES: CS(=O)(=O)N1CCN(C(=O)c2cnc3c([125I])cccc3c2-c2ccc(C3(C#N)CC3)cc2)CC1] | 1-(4-(8-(fluoro-¹²⁵I)-3-(4- (methylsulfonyl)piperazine-1- carbonyl)quinolin-4- yl)phenyl)cyclopropane-1- carbonitrile |
| PQM-H-018 | 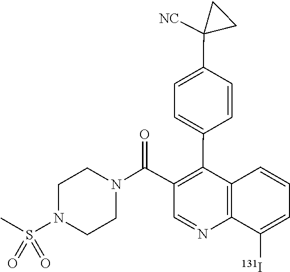 [SMILES: CS(=O)(=O)N1CCN(C(=O)c2cnc3c([131I])cccc3c2-c2ccc(C3(C#N)CC3)cc2)CC1] | 1-(4-(8-(fluoro-¹³¹I)-3-(4- (methylsulfonyl)piperazine-1- carbonyl)quinolin-4- yl)phenyl)cyclopropane-1- carbonitrile |
| PQM-H-019 | 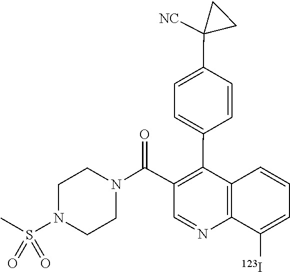 [SMILES: CS(=O)(=O)N1CCN(C(=O)c2cnc3c([123I])cccc3c2-c2ccc(C3(C#N)CC3)cc2)CC1] | 1-(4-(8-(fluoro-¹²³I)-3-(4- (methylsulfonyl)piperazine-1- carbonyl)quinolin-4- yl)phenyl)cyclopropane-1- carbonitrile |
| PQM-H-020 | 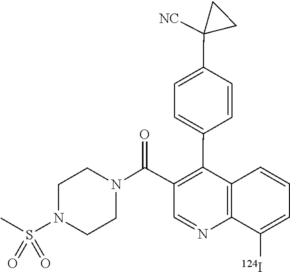 [SMILES: CS(=O)(=O)N1CCN(C(=O)c2cnc3c([124I])cccc3c2-c2ccc(C3(C#N)CC3)cc2)CC1] | 1-(4-(8-(fluoro-¹²⁴I)-3-(4- (methylsulfonyl)piperazine-1- carbonyl)quinolin-4- yl)phenyl)cyclopropane-1- carbonitrile |
| PQM-H-021 | 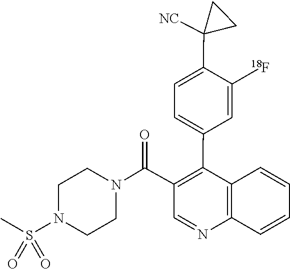 [SMILES: CS(=O)(=O)N1CCN(C(=O)c2cnc3ccccc3c2-c2ccc(C3(C#N)CC3)c([18F])c2)CC1] | 1-(2-(fluoro-¹⁸F)-4-(3-(4- (methylsulfonyl)piperazine-1- carbonyl)quinolin-4- yl)phenyl)cyclopropane-1- carbonitrile |
| PQM-H-022 | 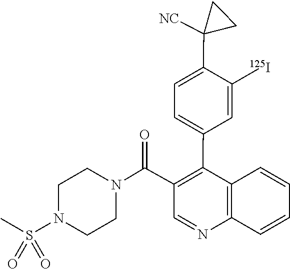 [SMILES: CS(=O)(=O)N1CCN(C(=O)c2cnc3ccccc3c2-c2ccc(C3(C#N)CC3)c([125I])c2)CC1] | 1-(2-(fluoro-¹²⁵I)-4-(3-(4- (methylsulfonyl)piperazine-1- carbonyl)quinolin-4- yl)phenyl)cyclopropane-1- carbonitrile |
| PQM-H-023 | 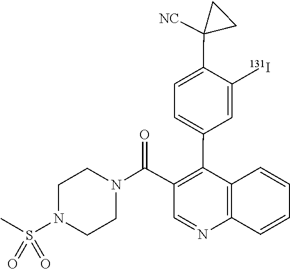 [SMILES: CS(=O)(=O)N1CCN(C(=O)c2cnc3ccccc3c2-c2ccc(C3(C#N)CC3)c([131I])c2)CC1] | 1-(2-(fluoro-¹³¹I)-4-(3-(4- (methylsulfonyl)piperazine-1- carbonyl)quinolin-4- yl)phenyl)cyclopropane-1- carbonitrile |
| PQM-H-024 | 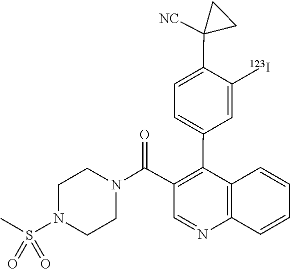 [SMILES: CS(=O)(=O)N1CCN(C(=O)c2cnc3ccccc3c2-c2ccc(C3(C#N)CC3)c([123I])c2)CC1] | 1-(2-(fluoro-¹²³I)-4-(3-(4- (methylsulfonyl)piperazine-1- carbonyl)quinolin-4- yl)phenyl)cyclopropane-1- carbonitrile |
| PQM-H-025 | 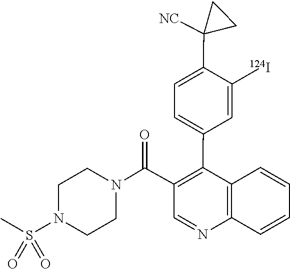 [SMILES: CS(=O)(=O)N1CCN(C(=O)c2cnc3ccccc3c2-c2ccc(C3(C#N)CC3)c([124I])c2)CC1] | 1-(2-(fluoro-¹²⁴I)-4-(3-(4- (methylsulfonyl)piperazine-1- carbonyl)quinolin-4- yl)phenyl)cyclopropane-1- carbonitrile |
| PQM-H-026 | 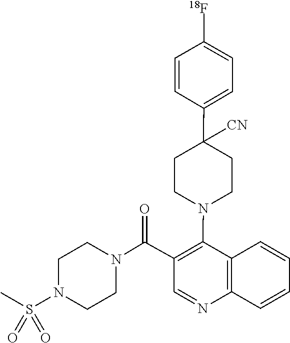 [SMILES: CS(=O)(=O)N1CCN(C(=O)c2cnc3ccccc3c2N2CCC(C#N)(c3ccc([18F])cc3)CC2)CC1] | 4-(4-(fluoro-¹⁸F)phenyl)-1-(3-(4- (methylsulfonyl)piperazine-1- carbonyl)quinolin-4-yl)piperidine-4- carbonitrile |
| PQM-H-027 | 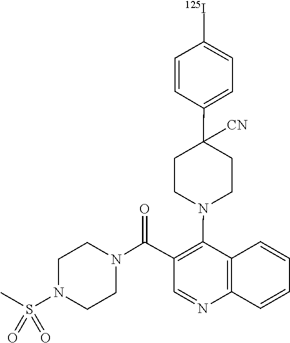 [SMILES: CS(=O)(=O)N1CCN(C(=O)c2cnc3ccccc3c2N2CCC(C#N)(c3ccc([125I])cc3)CC2)CC1] | 4-(4-(fluoro-¹²⁵I)phenyl)-1-(3-(4- (methylsulfonyl)piperazine-1- carbonyl)quinolin-4-yl)piperidine-4- carbonitrile |
| PQM-H-028 | 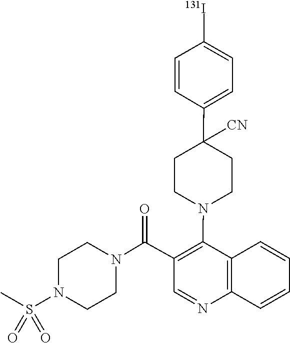 [SMILES: CS(=O)(=O)N1CCN(C(=O)c2cnc3ccccc3c2N2CCC(C#N)(c3ccc([131I])cc3)CC2)CC1] | 4-(4-(fluoro-¹³¹I)phenyl)-1-(3-(4- (methylsulfonyl)piperazine-1- carbonyl)quinolin-4-yl)piperidine-4- carbonitrile |
| PQM-H-029 | 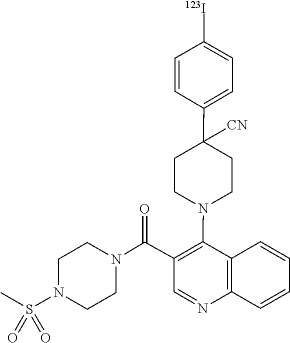 [SMILES: CS(=O)(=O)N1CCN(C(=O)c2cnc3ccccc3c2N2CCC(C#N)(c3ccc([123I])cc3)CC2)CC1] | 4-(4-(fluoro-¹²³I)phenyl)-1-(3-(4- (methylsulfonyl)piperazine-1- carbonyl)quinolin-4-yl)piperidine-4- carbonitrile |
| PQM-H-030 | 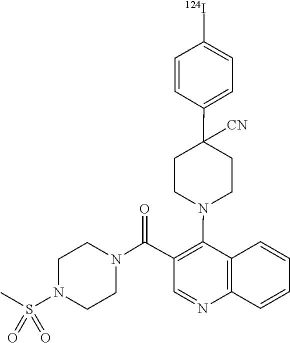 [SMILES: CS(=O)(=O)N1CCN(C(=O)c2cnc3ccccc3c2N2CCC(C#N)(c3ccc([124I])cc3)CC2)CC1] | 4-(4-(fluoro-¹²⁴I)phenyl)-1-(3-(4- (methylsulfonyl)piperazine-1- carbonyl)quinolin-4-yl)piperidine-4- carbonitrile |

(121) A compound according to (1), selected from compounds of the following formulae and pharmaceutically acceptable salts, hydrates, and solvates thereof:

| Cmpd No. | Structure | Name |
|---|---|---|
| PQM-H-001 | 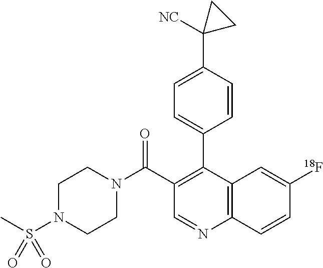 [SMILES: CS(=O)(=O)N1CCN(C(=O)c2cnc3ccc([18F])cc3c2-c2ccc(C3(C#N)CC3)cc2)CC1] | 1-(4-(6-(fluoro-¹⁸F)-3-(4- (methylsulfonyl)piperazine- 1-carbonyl)quinolin-4- yl)phenyl) cyclopropane-1- carbonitrile |
| PQM-H-002 | 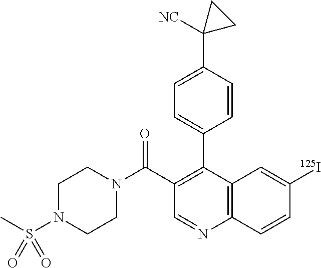 [SMILES: CS(=O)(=O)N1CCN(C(=O)c2cnc3ccc([125I])cc3c2-c2ccc(C3(C#N)CC3)cc2)CC1] | 1-(4-(6-(iodo-¹²⁵I)-3-(4- (methylsulfonyl)piperazine- 1-carbonyl)quinolin-4- yl)phenyl)cyclopropane-1- carbonitrile |
| PQM-H-003 | 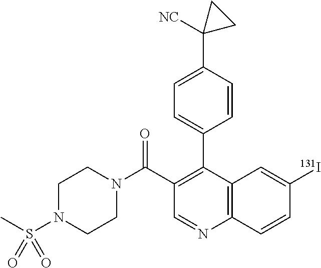 [SMILES: CS(=O)(=O)N1CCN(C(=O)c2cnc3ccc([131I])cc3c2-c2ccc(C3(C#N)CC3)cc2)CC1] | 1-(4-(6-(iodo-¹³¹I)-3-(4- (methylsulfonyl)piperazine- 1-carbonyl)quinolin-4- yl)phenyl)cyclopropane-1- carbonitrile |
| PQM-H-004 | 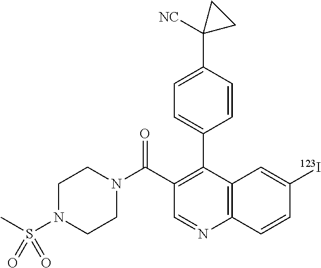 [SMILES: CS(=O)(=O)N1CCN(C(=O)c2cnc3ccc([123I])cc3c2-c2ccc(C3(C#N)CC3)cc2)CC1] | 1-(4-(6-(iodo-¹²³I)-3-(4- (methylsulfonyl)piperazine- 1-carbonyl)quinolin-4- yl)phenyl)cyclopropane-1- carbonitrile |
| PQM-H-005 | 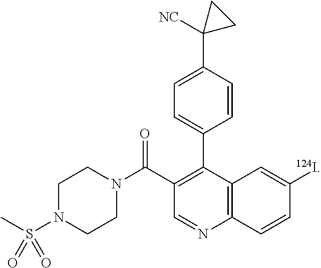 | 1-(4-(6-(iodo-¹²⁴I)-3-(4- (methylsulfonyl)piperazine- 1-carbonyl)quinolin-4- yl)phenyl)cyclopropane-1- carbonitrile |
| PQM-H-006 | 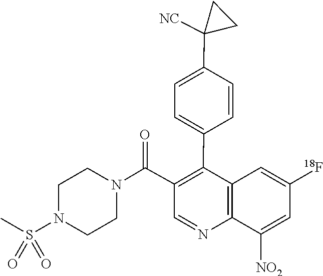 [SMILES: CS(=O)(=O)N1CCN(C(=O)c2cnc3c([N+](=O)[O-])cc([18F])cc3c2-c2ccc(C3(C#N)CC3)cc2)CC1] | 1-(4-(6-(fluoro-¹⁸F)-3-(4- (methylsulfonyl)piperazine- 1-carbonyl)-8-nitroquinolin- 4-yl)phenyl)cyclopropane- 1-carbonitrile |
| PQM-H-007 | 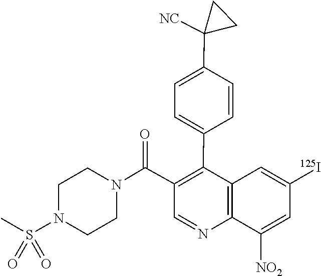 [SMILES: CS(=O)(=O)N1CCN(C(=O)c2cnc3c([N+](=O)[O-])cc([125I])cc3c2-c2ccc(C3(C#N)CC3)cc2)CC1] | 1-(4-(6-(iodo-¹²⁵I)-3-(4- (methylsulfonyl)piperazine- 1-carbonyl)-8-nitroquinolin- 4-yl)phenyl)cyclopropane- 1-carbonitrile |
| PQM-H-008 | 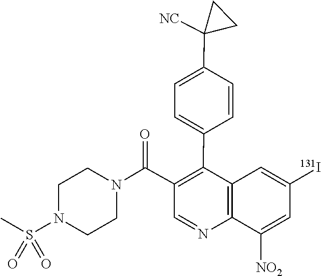 [SMILES: CS(=O)(=O)N1CCN(C(=O)c2cnc3c([N+](=O)[O-])cc([131I])cc3c2-c2ccc(C3(C#N)CC3)cc2)CC1] | 1-(4-(6-(iodo-¹³¹I)-3-(4- (methylsulfonyl)piperazine- 1-carbonyl)-8-nitroquinolin- 4-yl)phenyl)cyclopropane- 1-carbonitrile |
| PQM-H-009 | 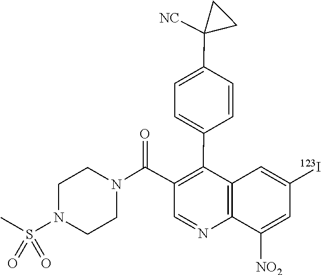 [SMILES: CS(=O)(=O)N1CCN(C(=O)c2cnc3c([N+](=O)[O-])cc([123I])cc3c2-c2ccc(C3(C#N)CC3)cc2)CC1] | 1-(4-(6-(iodo-¹²³I)-3-(4- (methylsulfonyl)piperazine- 1-carbonyl)-8-nitroquinolin- 4-yl)phenyl)cyclopropane- 1-carbonitrile |
| PQM-H-010 | 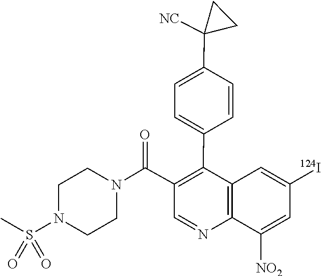 [SMILES: CS(=O)(=O)N1CCN(C(=O)c2cnc3c([N+](=O)[O-])cc([124I])cc3c2-c2ccc(C3(C#N)CC3)cc2)CC1] | 1-(4-(6-(iodo-¹²⁴I)-3-(4- (methylsulfonyl)piperazine- 1-carbonyl)-8-nitroquinolin- 4-yl)phenyl)cyclopropane- 1-carbonitrile |
| PQM-H-016 | 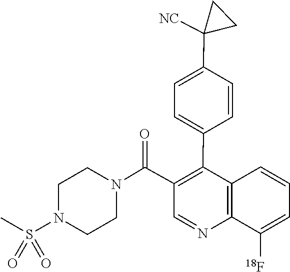 [SMILES: CS(=O)(=O)N1CCN(C(=O)c2cnc3c([18F])cccc3c2-c2ccc(C3(C#N)CC3)cc2)CC1] | 1-(4-(8-(fluoro-¹⁸F)-3-(4- (methylsulfonyl)piperazine- 1-carbonyl)quinolin-4- yl)phenyl)cyclopropane-1- carbonitrile |
| PQM-H-017 | 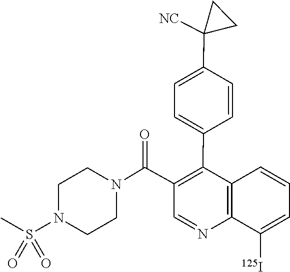 [SMILES: CS(=O)(=O)N1CCN(C(=O)c2cnc3c([125I])cccc3c2-c2ccc(C3(C#N)CC3)cc2)CC1] | 1-(4-(8-(fluoro-¹²⁵I)-3-(4- (methylsulfonyl)piperazine- 1-carbonyl)quinolin-4- yl)phenyl)cyclopropane-1- carbonitrile |
| PQM-H-018 | 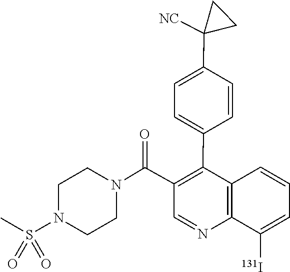 [SMILES: CS(=O)(=O)N1CCN(C(=O)c2cnc3c([131I])cccc3c2-c2ccc(C3(C#N)CC3)cc2)CC1] | 1-(4-(8-(fluoro-¹³¹I)-3-(4- (methylsulfonyl)piperazine- 1-carbonyl)quinolin-4- yl)phenyl)cyclopropane-1- carbonitrile |
| PQM-H-019 | 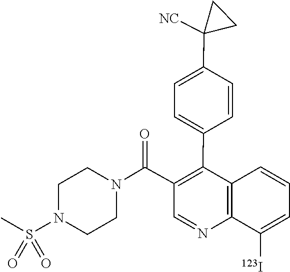 [SMILES: CS(=O)(=O)N1CCN(C(=O)c2cnc3c([123I])cccc3c2-c2ccc(C3(C#N)CC3)cc2)CC1] | 1-(4-(8-(fluoro-¹²³I)-3-(4- (methylsulfonyl)piperazine- 1-carbonyl)quinolin-4- yl)phenyl)cyclopropane-1- carbonitrile |
| PQM-H-020 | 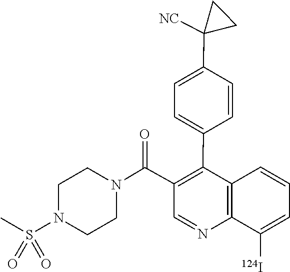 [SMILES: CS(=O)(=O)N1CCN(C(=O)c2cnc3c([124I])cccc3c2-c2ccc(C3(C#N)CC3)cc2)CC1] | 1-(4-(8-(fluoro-¹²⁴I)-3-(4- (methylsulfonyl)piperazine- 1-carbonyl)quinolin-4- yl)phenyl) cyclopropane-1- carbonitrile |
| PQM-H-026 | 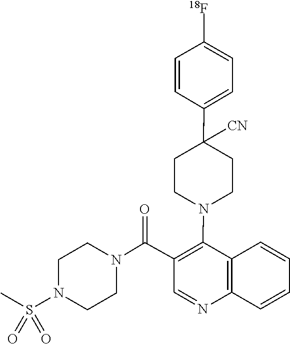 [SMILES: CS(=O)(=O)N1CCN(C(=O)c2cnc3ccccc3c2N2CCC(C#N)(c3ccc([18F])cc3)CC2)CC1] | 4-(4-(fluoro-¹⁸F)phenyl)-1- (3-(4- (methylsulfonyl)piperazine- 1-carbonyl)quinolin-4- yl)piperidine-4-carbonitrile |

(122) A compound according to (1), selected from compounds of the following formulae and pharmaceutically acceptable salts, hydrates, and solvates thereof:

| Cmpd No. | Structure | Name |
|---|---|---|
| PQM-H-001 | 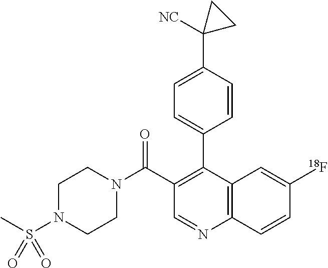 [SMILES: CS(=O)(=O)N1CCN(C(=O)c2cnc3ccc([18F])cc3c2-c2ccc(C3(C#N)CC3)cc2)CC1] | 1-(4-(6-(fluoro-¹⁸F)-3-(4- (methylsulfonyl)piperazine-1- carbonyl)quinolin-4- yl)phenyl)cyclopropane-1- carbonitrile |
| PQM-H-002 | 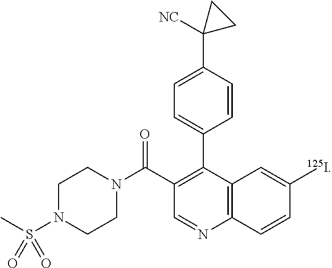 | 1-(4-(6-(iodo-¹²⁵I)-3-(4- (methylsulfonyl)piperazine-1- carbonyl)quinolin-4- yl)phenyl)cyclopropane-1- carbonitrile |

(123) A compound according to (1), that is 1-(4-(6-(fluoro-¹⁸F)-3-(4-(methylsulfonyl)piperazine-1-carbonyl)quinolin-4-yl)phenyl)cyclopropane-1-carbonitrile (PQM-H-001), or a pharmaceutically acceptable salt, hydrate, or solvate thereof.

(124) A compound according to (1), that is 1-(4-(6-(iodo-¹²⁵I)-3-(4-(methylsulfonyl)piperazine-1-carbonyl)quinolin-4-yl)phenyl)cyclopropane-1-carbonitrile (PQM-H-002), or a pharmaceutically acceptable salt, hydrate, or solvate thereof.

(125) A compound according to (1), that is 1-(4-(6-(fluoro-¹⁸F)-3-(4-(methylsulfonyl)piperazine-1-carbonyl)-8-nitroquinolin-4-yl)phenyl)cyclopropane-1-carbonitrile (PQM-H-006), or a pharmaceutically acceptable salt, hydrate, or solvate thereof.

(126) A compound according to (1), that is 1-(4-(6-(iodo-¹²⁵I)-3-(4-(methylsulfonyl)piperazine-1-carbonyl)-8-nitroquinolin-4-yl)phenyl)cyclopropane-1-carbonitrile (PQM-H-007), or a pharmaceutically acceptable salt, hydrate, or solvate thereof.

(127) A compound according to (1), that is 1-(4-(8-(fluoro-¹⁸F)-3-(4-(methylsulfonyl)piperazine-1-carbonyl)quinolin-4-yl)phenyl)cyclopropane-1-carbonitrile (PQM-H-016), or a pharmaceutically acceptable salt, hydrate, or solvate thereof.

(128) A compound according to (1), that is 1-(4-(8-(fluoro-¹²⁵I)-3-(4-(methylsulfonyl)piperazine-1-carbonyl)quinolin-4-yl)phenyl)cyclopropane-1-carbonitrile (PQM-H-017), or a pharmaceutically acceptable salt, hydrate, or solvate thereof.

(129) A compound according to (1), that is 4-(4-(fluoro-¹⁸F)phenyl)-1-(3-(4-(methylsulfonyl)piperazine-1-carbonyl)quinolin-4-yl)piperidine-4-carbonitrile (PQM-H-026), or a pharmaceutically acceptable salt, hydrate, or solvate thereof.

### Radiolabelled Compound Precursors

The radiolabelled (PQM-H) compounds described herein undergo radioactive decay. Consequently, the PQM-H compounds cannot be stored for prolonged periods of time without a reduction in the radioactivity of the sample. Moreover, storage and transportation of the PQM-H compounds poses a safety risk to those working with the compounds due to exposure to radioactive decay.

Accordingly, it is convenient to store and transport a PQM-H compound as a synthetic precursor (i.e., a PQM-P compound). A PQM-H compound can then be produced from an appropriate PQM-P compound at the time and place required for use via a short synthetic transformation. In this way, exposure of third parties to the radioactivity produced by PQM-H compounds is minimized, and the amount of radiolabelled compound that can be provided to a subject in need thereof can be maximised. In practical terms, the PQM-P compounds are very important for the expected mode of use of the PQM-H compounds.

Accordingly, another aspect of the present invention relates to certain compounds which are synthetic precursors to the PQM-H compounds, wherein the radioisotope is replaced with a leaving group (e.g., a —BPin group).

Thus, another aspect of the present invention is a compound of the following formula, or a pharmaceutically acceptable salt, hydrate, or solvate thereof, wherein —R¹⁴, -L¹¹-, —R^{1L}, —R¹⁶, —R¹⁷ and —R¹⁸ are as defined herein (for convenience, collectively referred to herein as “PQM-P” compounds”):

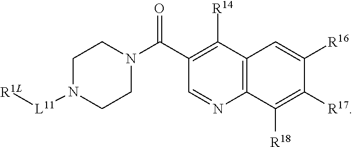

(130) A compound according to formula (Ia):

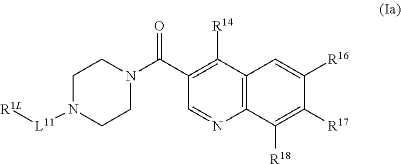

or a salt, hydrate, or solvate thereof;
wherein:
   —R¹⁴ is

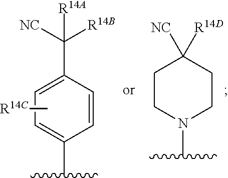

—R^{14A} and —R^{14B} taken together with the carbon atom to which they are attached form a C₃₋₅ cycloalkyl; or —R^{14A} and —R^{14B} are each independently C₁₋₃ alkyl;
—R^{14C} is —H, —F, or —R^{X14C};
—R^{14D} is phenyl, optionally substituted with:
   a) one group —R^{X14D}; and/or
   b) one or more —F;
L¹¹- is independently selected from —S(O)₂— and —C(O)—;
—R^{1L} is independently selected from C₁₋₆ alkyl and cyclopropyl;
—R¹⁶ is independently selected from —H, —F, —I, —R^{X16}, and —NO₂;
—R¹⁷ is independently selected from —H, —F, —I, —R^{X17}, and —NO₂;
—R¹⁸ is independently selected from —H, —F, —I, —Rx¹⁸, and —NO₂;
each of —R^{X14C}, —R^{X14D}, —R^{X16}, —R^{X17}, and —R^{X18} is, independently, a leaving group;
the compound comprises only one group selected from —R^{X14C}, —R^{X14D}, —R^{X16}, —R^{X17}, and —R^{X18};
and at least one of —R¹⁶, —R¹⁷, and —R¹⁸ is —H.

(131) The compound according to (130), or a salt, hydrate, or solvate thereof, wherein the compound is a compound according to formula (Ia):

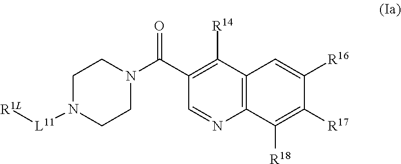

or a salt, hydrate, or solvate thereof;
wherein:
   —R¹⁴ is

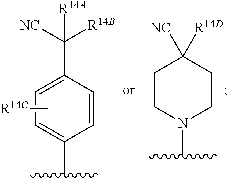

—R^{14A} and —R^{14B} taken together with the carbon atom to which they are attached form a C₃₋₅ cycloalkyl; or —R^{14A} and —R^{14B} are each independently C₁₋₃ alkyl;
—R^{14C} is —H or —F;
—R^{14D} is phenyl, optionally substituted with one of more —F;
L¹¹- is independently selected from —S(O)₂— and —C(O)—;
—R^{1L} is independently selected from C₁₋₆ alkyl and cyclopropyl;
—R¹⁶ is independently selected from —H, —F, —I, a leaving group, and —NO₂;
—R¹⁷ is independently selected from —H, —F, —I, a leaving group, and —NO₂;
—R¹⁸ is independently selected from —H, —F, —I, a leaving group, and —NO₂;
wherein, one of —R¹⁶, —R¹⁷, and —R¹⁸ is a leaving group, and the others of —R¹⁶, —R¹⁷, and —R¹⁸ are not leaving groups; and at least one of —R¹⁶, —R¹⁷, and —R¹⁸ is —H.

### The Group —R¹⁴

(132) The compound according to (130) or (131), or a salt, hydrate, or solvate thereof, wherein:
—R¹⁴ is

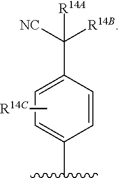

(133) The compound according to (130) or (131), or a salt, hydrate, or solvate thereof, wherein:
—R¹⁴ is

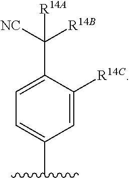

(134) The compound according to (130) or (131), or a salt, hydrate, or solvate thereof, wherein:
—R¹⁴ is

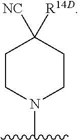

The Groups —R^{14A} and —R^{14B}

(135) The compound according to any one of (130)-(133), or a salt, hydrate, or solvate thereof, wherein:
—R^{14A} and —R^{14B}, taken together with the carbon atom to which they are attached form a C₃₅ cycloalkyl.

(136) The compound according to any one of (130)-(133), or a salt, hydrate, or solvate thereof, wherein:
—R^{14A} and —R^{14B}, taken together with the carbon atom to which they are attached form cyclopropyl.

(137) The compound according to any one of (130)-(133), or a salt, hydrate, or solvate thereof, wherein:
—R^{14A} and —R^{14B}, are each independently C₁₋₃ alkyl.

(138) The compound according to any one of (130)-(133), or a salt, hydrate, or solvate thereof, wherein:
—R^{14A} and —R^{14B}, are each methyl.

### The Group —R^{14C}

(139) The compound according to any one of (130)-(133) and (135)-(138), as applicable, or a salt, hydrate, or solvate thereof, wherein:
—R^{14C} is —H or —R^{X14C}

(140) The compound according to any one of (130)-(133) and (135)-(138), or a salt, hydrate, or solvate thereof, wherein:
—R^{14C} is —H or —F.

(141) The compound according to any one of (130)-(133) and (135)-(138), or a salt, hydrate, or solvate thereof, wherein:
—R^{14C} is —H.

(142) The compound according to any one of (130)-(133) and (135)-(138), or a salt, hydrate, or solvate thereof, wherein:
—R^{14C} is —F.

(143) The compound according to any one of (130)-(133) and (135)-(138), as applicable, or a salt, hydrate, or solvate thereof, wherein:
—R^{14C} is —R^{X14C}.

### The Group —R^{14D}

(144) The compound according to any one of (130), (131) or (134), or a salt, hydrate, or solvate thereof, wherein:
—R^{14D} is phenyl optionally substituted with one or more —F.

(145) The compound according to any one of (130), (131) or (134), or a salt, hydrate, or solvate thereof, wherein:
—R^{14D}, is phenyl.

(146) The compound according to any one of (130), (131) or (134), or a salt, hydrate, or solvate thereof, wherein:
—R^{14D} is phenyl substituted with —F.

(147) The compound according to (130) or (134), or a salt, hydrate, or solvate thereof, wherein:
—R^{14D} is phenyl optionally substituted with one —R^{X14D}.

(148) The compound according to (130) or (134), or a salt, hydrate, or solvate thereof, wherein:
—R^{14D} is phenyl substituted with one —R^{X14D}.

(149) The compound according to (130) or (134), or a salt, hydrate, or solvate thereof, wherein:
—R^{14D} is

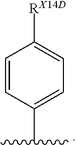

### The Group -L¹¹-

(150) The compound according to any one of (130)-(149), or a salt, hydrate, or solvate thereof, wherein:
-L¹¹- is —S(O)₂—.

(151) The compound according to any one of (130)-(149), or a salt, hydrate, or solvate thereof, wherein:
-L¹¹- is —C(O)—.

### The Group —R^{1L}

(152) The compound according to any one of (130)-(151), or a salt, hydrate, or solvate thereof, wherein:
—R^{L}— is C₁₋₆ alkyl.

(153) The compound according to any one of (130)-(151), or a salt, hydrate, or solvate thereof, wherein:
—R^{1L}— is C₁₋₃ alkyl.

(154) The compound according to any one of (130)-(151), or a salt, hydrate, or solvate thereof, wherein:
—R^{1L}— is methyl.

(155) The compound according to any one of (130)-(151), or a salt, hydrate, or solvate thereof, wherein:
—R^{1L}— is cyclopropyl.

### The Group —R¹⁶

(156) The compound according to any one of (130)-(155), as applicable, or a salt, hydrate, or solvate thereof, wherein:
—R¹⁶ is selected from —H, —F, —I, a leaving group, and —NO₂.

(157) The compound according to any one of (130)-(155), as applicable, or a salt, hydrate, or solvate thereof, wherein:
—R¹⁶ is selected from —H, —R^{X16}, and —NO₂.

(158) The compound according to any one of (130)-(155), as applicable, or a salt, hydrate, or solvate thereof, wherein:
—R¹⁶ is selected from —H and —R^{X16}

(159) The compound according to any one of (130)-(155), or a salt, hydrate, or solvate thereof, wherein:
—R¹⁶ is a not a leaving group and is selected from —H, —F, —I, and —NO₂.

(160) The compound according to any one of (130)-(155), or a salt, hydrate, or solvate thereof, wherein:
—R¹⁶ is a not a leaving group and is selected from —H, and —NO₂.

(161) The compound according to any one of (130)-(155), or a salt, hydrate, or solvate thereof, wherein:
—R¹⁶ is a not a leaving group and is selected from —H, —F, and —I.

(162) The compound according to any one of (130)-(155), or a salt, hydrate, or solvate thereof, wherein:
—R¹⁶ is a not a leaving group and is selected from —H, and —F.

(163) The compound according to any one of (130)-(155), or a salt, hydrate, or solvate thereof, wherein:
—R¹⁶ is a not a leaving group and is —H.

(164) The compound according to any one of (130)-(155), or a salt, hydrate, or solvate thereof, wherein:
—R¹⁶ is the leaving group, for example, —R¹⁶ is —R^{X16}.

### The Group —R¹⁷

(165) The compound according to any one of (130)-(164), as applicable, or a salt, hydrate, or solvate thereof, wherein:
—R¹⁷ is selected from —H, —F, —I, a leaving group, and —NO₂.

(166) The compound according to any one of (130)-(164), as applicable, or a salt, hydrate, or solvate thereof, wherein:
—R¹⁷ is selected from —H, —R^{X17}, and —NO₂.

(167) The compound according to any one of (130)-(164), as applicable, or a salt, hydrate, or solvate thereof, wherein:
—R¹⁷ is selected from —H and —R^{X17}.

(168) The compound according to any one of (130)-(164), or a salt, hydrate, or solvate thereof, wherein:
—R¹⁷ is a not a leaving group and is selected from —H, —F, —I, and —NO₂.

(169) The compound according to any one of (130)-(164), or a salt, hydrate, or solvate thereof, wherein:
—R¹⁷ is a not a leaving group and is selected from —H, and —NO₂.

(170) The compound according to any one of (130)-(164), or a salt, hydrate, or solvate thereof, wherein:
—R¹⁷ is a not a leaving group and is selected from —H, —F, and —I.

(171) The compound according to any one of (130)-(164), or a salt, hydrate, or solvate thereof, wherein:
—R¹⁷ is a not a leaving group and is selected from —H, and —F.

(172) The compound according to any one of (130)-(164), or a salt, hydrate, or solvate thereof, wherein:
—R¹⁷ is a not a leaving group and is —H.

(173) The compound according to any one of (130)-(164), as applicable, or a salt, hydrate, or solvate thereof, wherein:
—R¹⁷ is the leaving group, for example, —R¹⁷ is —R^{X17}.

### The Group —R¹⁸

(174) The compound according to any one of (130)-(173), as applicable, or a salt, hydrate, or solvate thereof, wherein:
—R¹⁸ is selected from —H, —F, —I, a leaving group, and —NO₂.

(175) The compound according to any one of (130)-(173), as applicable, or a salt, hydrate, or solvate thereof, wherein:
—R¹⁸ is selected from —H, —R^{X18}, and —NO₂.

(176) The compound according to any one of (130)-(173), as applicable, or a salt, hydrate, or solvate thereof, wherein:
—R¹⁸ is selected from —H and —R^{X18}.

(177) The compound according to any one of (130)-(173), or a salt, hydrate, or solvate thereof, wherein:
—R¹⁸ is a not a leaving group and is selected from —H, —F, —I, and —NO₂.

(178) The compound according to any one of (130)-(173), or a salt, hydrate, or solvate thereof, wherein:
—R¹⁸ is a not a leaving group and is selected from —H, and —NO₂.

(179) The compound according to any one of (130)-(173), or a salt, hydrate, or solvate thereof, wherein:
—R¹⁸ is a not a leaving group and is selected from —H, —F, and —I.

(180) The compound according to any one of (130)-(173), or a salt, hydrate, or solvate thereof, wherein:
—R¹⁸ is a not a leaving group and is selected from —H, and —F.

(181) The compound according to any one of (130)-(173), or a salt, hydrate, or solvate thereof, wherein:
—R¹⁸ is a not a leaving group and is —H.

(182) The compound according to any one of (130)-(173), as applicable, or a salt, hydrate, or solvate thereof, wherein:
—R¹⁸ is the leaving group, for example, —R¹⁸ is —R^{X18}

### The Leaving Group

As used herein, the term “leaving group” describes a group suitable to act as such in reactions to halogenate an aryl ring with one of the halogen radioisotopes described herein (e.g., ¹⁸F, ¹²³I, ¹²⁴I, ¹²⁵I, ¹³¹I, and ²¹¹At). For example, the leaving groups described herein are suitable for use in nucleophilic halogenation reactions, such as a copper-catalysed nucleophilic halogenation including copper-catalysed fluorination and copper-catalysed iodination. The term leaving group is defined in: IUPAC. Compendium of Chemical Terminology, 2nd ed. (the “Gold Book”). Compiled by A. D. McNaught and A. Wilkinson. Blackwell Scientific Publications, Oxford (1997). Online version (2019-) created by S. J. Chalk. ISBN 0-9678550-9-8. https://doi.org/10.1351/goldbook. Suitable leaving groups for the PQM-P compounds described herein include: boronic acid esters, boronic acid, alkylsulfonates, haloalkylsulfonates, arylsulfonates, trialkylamines, organostannanes, aryliodonium salts, and arylsulfonium salts. In some embodiments, the leaving group is a nucleofuge. In some embodiments, a compound substituted with a leaving group described herein has a relative reactivity in a given nucleophilic substitution reaction which is higher than that of the same compound wherein the leaving group is replaced with iodide.

Just as the PQM-H compounds comprise only one radioisotope, so too, the PQM-P compounds comprise only one leaving group. That is the PQM-P compounds described herein comprise a leaving group at one, and no more than one substituent position. More specifically, a PQM-P compound comprises only one group selected from —R^{X14C}, —R^{X14D}, —R^{X16}, —R^{X17}, and —R^{X18} (each of which is defined as a leaving group). That is, in the PQM-P compounds described herein one of —R^{X14C}, —R^{X14D}, —R^{X16}, —R^{X17}, and —R^{X18} is present and the others of —R^{X14C}, —R^{X14D}, —R^{X16}, —R^{X17}, and —R^{X18} are not present.

(183) The compound according to any one of (130)-(182), or a salt, hydrate, or solvate thereof, wherein:
the leaving group is selected from boronic acid esters, boronic acid, alkylsulfonates, haloalkylsulfonates, arylsulfonates, trialkylamines, organostannanes, aryliodonium salts, and arylsulfonium salts.

(184) The compound according to any one of (130)-(182), or a salt, hydrate, or solvate thereof, wherein:
the leaving group is selected from boronic acid esters, boronic acid, trialkylamines, organostannanes, aryliodonium salts, and arylsulfonium salts.

(185) The compound according to any one of (130)-(182), or a salt, hydrate, or solvate thereof, wherein:
the leaving group is selected from boronic acid esters, boronic acid, aryliodonium salts, and arylsulfonium salts.

(186) The compound according to any one of (130)-(182), or a salt, hydrate, or solvate thereof, wherein:
the leaving group is selected from boronic acid esters, and boronic acid.

(187) The compound according to any one of (130)-(182), or a salt, hydrate, or solvate thereof, wherein:
the leaving group is selected from boronic acid esters.

(188) The compound according to any one of (183)-(187), or a salt, hydrate, or solvate thereof, wherein:
the boronic acid ester is selected from boronic acid pinacol ester (—BPin), boronic acid neopentylglycol ester (—Bneop), boronic acid MIDA ester (—BMIDA), boronic acid catechol ester (—BCat), boronic acid hexylene glycol ester (—Bhex), and boronic acid 1,3-propanediol ester (—Bprop).

(189) The compound according to any one of (183)-(187), or a salt, hydrate, or solvate thereof, wherein:
the boronic acid ester is selected from boronic acid pinacol ester (—BPin) and boronic acid catechol ester (—BCat).

(190) The compound according to any one of (183)-(187), or a salt, hydrate, or solvate thereof, wherein:
the boronic acid ester is boronic acid pinacol ester (—BPin).

(191) The compound according to (183), or a salt, hydrate, or solvate thereof, wherein:
the alkylsulfonate is mesylate.

(192) The compound according to (183), or a salt, hydrate, or solvate thereof, wherein:
the haloalkylsulfonate is triflate or perfluorobutylsulfonate.

(193) The compound according to (183), or a salt, hydrate, or solvate thereof, wherein:
the haloalkylsulfonate is triflate.

(194) The compound according to (183), or a salt, hydrate, or solvate thereof, wherein:
the arylsulfonate is tosylate.

(195) The compound according to (183) or (184), or a salt, hydrate, or solvate thereof, wherein:
the trialkylamine is —N⁺(C₁₋₆alkyl)₃.

(196) The compound according to (183) or (184), or a salt, hydrate, or solvate thereof, wherein:
the trialkylamine is —N⁺Me₃.

(197) The compound according to (183) or (184), or a salt, hydrate, or solvate thereof, wherein:
the organostannane is —Sn(C₁₋₆alkyl)₃.

(198) The compound according to (183) or (184), or a salt, hydrate, or solvate thereof, wherein:
the organostannane is —SnMe₃.

(199) The compound according to any one of (183)-(185), or a salt, hydrate, or solvate thereof, wherein:
the aryliodonium salt is -[PhI]⁺BF₄⁻.

(200) The compound according to any one of (183)-(185), or a salt, hydrate, or solvate thereof, wherein:
the arylsulfonium salt is -[Ph₂S]⁺TfO⁻.

(201) The compound according to any one of (130)-(182), or a salt, hydrate, or solvate thereof, wherein:
the leaving group is selected from: —BPin, —B(OH)₂, -[PhI]⁺BF₄⁻, -[Ph₂S]⁺TfO⁻, —SnMe₃, and —N⁺Me₃.

(202) The compound according to any one of (130)-(182), or a salt, hydrate, or solvate thereof, wherein:
the leaving group is selected from: —BPin and —B(OH)₂.

(203) The compound according to any one of (130)-(182), or a salt, hydrate, or solvate thereof, wherein:
the leaving group is —BPin.

(204) The compound according to any one of (130)-(182), or a salt, hydrate, or solvate thereof, wherein:
the leaving group is —B(OH)₂.

(205) The compound according to any one of (130)-(204), as applicable, or a salt, hydrate, or solvate thereof, wherein:
—R¹⁶, —R¹⁷, or —R¹⁸ is the leaving group, for example, —R¹⁶ is —R^{X16}, —R¹⁷ is —R^{X17}, or —R¹⁸ is —R^{X18}.

(206) The compound according to any one of (130)-(204), as applicable, or a salt, hydrate, or solvate thereof, wherein:
—R¹⁶ or —R¹⁷ is the leaving group, for example, —R¹⁶ is —R^{X18} or —R¹⁷ is —R^{X17}.

(207) The compound according to any one of (130)-(204), as applicable, or a salt, hydrate, or solvate thereof, wherein:
—R¹⁶ or —R¹⁸ is the leaving group, for example, —R¹⁶ is —R^{X16} or —R¹⁸ is —R^{X18}.

For the avoidance of doubt, the term “—BPin”, as used herein, refers to a boronic acid pinacol ester group, which may act as a leaving group. That is, the group:

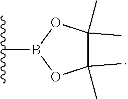 [SMILES: *B1OC(C)(C)C(C)(C)O1]

For the avoidance of doubt, the term “-[PhI]⁺BF₄⁻”, as used herein, refers to a phenyliodonium tetrafluoroborate group, which may act as a leaving group. That is, the group:

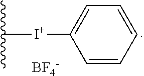

For the avoidance of doubt, the term “-[Ph₂S]⁺TfO⁻”, as used herein, refers to a diarylsulfonium triflate group, which may act as a leaving group. That is, the group:

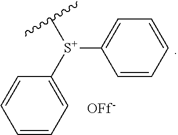 [SMILES: *[S+](c1ccccc1)c1ccccc1]

For the avoidance of doubt, each of the groups —R^{X14C}, —R^{X14D}, —R^{X16}, —R^{X17}, and —R^{X18} may be defined separately and independently. Accordingly, each of the embodiments relating to “the leaving group” described hereinabove, may be applied to the groups —R^{X14C}, —R^{X14D}, —R^{X16}, —R^{X17}, and —R^{X18} separately and independently. In one embodiment, —R^{X14C} is a leaving group that is selected from boronic acid esters, such as —BPin. In one embodiment, —R^{X14D} is a leaving group that is selected from boronic acid esters, such as —BPin. In one embodiment, —R^{X16} is a leaving group that is selected from boronic acid esters, such as —BPin. In one embodiment, —R^{X17} is a leaving group that is selected from boronic acid esters, such as —BPin. In one embodiment, —R^{X18} is a leaving group that is boronic acid (i.e., —B(OH)₂).

### Some Preferred Combinations

(208) The compound according to any one of (130)-(155), or a salt or solvate thereof, wherein:
—R¹⁶ is a leaving group, for example, —R¹⁶ is —R^{X16}; and
—R¹⁷ is —H.

(209) The compound according to any one of (130)-(155), or a salt, hydrate, or solvate thereof, wherein:
—R¹⁶ is a leaving group, for example, —R¹⁶ is —R^{X16};
—R¹⁷ is —H; and —R¹⁸ is —H or NO₂.

(210) The compound according to any one of (130)-(155), or a salt, hydrate, or solvate thereof, wherein:
—R¹⁶ is a leaving group, for example, —R¹⁶ is —R^{X16};
—R¹⁷ is —H; —R¹⁸ is —H or NO₂; and
the leaving group is —BPin.

(211) The compound according to any one of (130)-(155), or a salt, hydrate, or solvate thereof, wherein:
—R¹⁷ is a leaving group, for example, —R¹⁷ is —R^{X17}; and
—R¹⁶ is —H; —R¹⁸ is —H.

(212) The compound according to any one of (130)-(155), or a salt, hydrate, or solvate thereof, wherein:
—R¹⁷ is a leaving group, for example, —R¹⁷ is —R^{X17};
—R¹⁶ is —H; —R¹⁸ is —H; and
the leaving group is —BPin.

(213) The compound according to any one of (130)-(155), or a salt, hydrate, or solvate thereof, wherein:
—R¹⁶ is —H or —NO₂; —R¹⁷ is —H; and
—R¹⁸ is a leaving group, for example, —R¹⁸ is —R^{X18}.

(214) The compound according to any one of (130)-(155), or a salt, hydrate, or solvate thereof, wherein:
—R¹⁶ is —H or —NO₂; —R¹⁷ is —H;
—R¹⁸ is a leaving group, for example, —R¹⁸ is —R^{X18}; and the leaving group is —BPin.

(215) The compound according to any one of (130)-(155), or a salt, hydrate, or solvate thereof, wherein:
—R^{14C}, if present, is —H, —F, or —R^{X14C};
—R^{14D}, if present, is phenyl, optionally substituted with one group —R^{X14D};
—R¹⁶ is independently selected from —H, —F, —I, and —NO₂;
—R¹⁷ is independently selected from —H, —F, —I, and —NO₂; and
—R¹⁸ is independently selected from —H, —F, —I, and —NO₂.

(216) The compound according to any one of (130)-(155), or a salt, hydrate, or solvate thereof, wherein:
—R^{14C}, if present, is —H, —F, or —R^{X14C};
—R^{14D}, if present, is phenyl, optionally substituted with one group —R^{X14D};
—R¹⁴ is independently selected from —H, —F, —I, and —NO₂;
—R¹⁴ is independently selected from —H, —F, —I, and —NO₂;
—R¹⁴ is independently selected from —H, —F, —I, and —NO₂;
—R^{X14C} is a leaving group selected from boronic acid esters, boronic acid, trialkylamines, organostannanes, aryliodonium salts, and arylsulfonium salts; and
—R^{X14D} is a leaving group selected from boronic acid esters, boronic acid, trialkylamines, organostannanes, aryliodonium salts, and arylsulfonium salts.

(217) The compound according to any one of (130)-(155), or a salt, hydrate, or solvate thereof, wherein:
—R^{14C}, if present, is —H, —F, or —R^{X14C};
—R^{14D}, if present, is phenyl, optionally substituted with one group —R^{X14D};
—R¹⁶ is independently selected from —H, —F, —I, and —NO₂;
—R¹⁷ is independently selected from —H, —F, —I, and —NO₂;
—R¹⁸ is independently selected from —H, —F, —I, and —NO₂;
—R^{X14C} is a leaving group that is selected from —BPin and —B(OH)₂; and
—R^{X14D} is a leaving group that is selected from —BPin and —B(OH)₂.

(218) The compound according to (130), or a salt, hydrate, or solvate thereof, wherein:
—R¹⁴ is

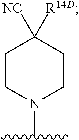

and
—R^{14D} is phenyl substituted with one group —R^{X14D}.

(219) The compound according to (130), or a salt, hydrate, or solvate thereof, wherein:
—R¹⁴ is

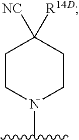

—R^{14D} is phenyl substituted with one group —R^{X14D};

(220) The compound according to (130), or a salt, hydrate, or solvate thereof, wherein:
—R¹⁴ is

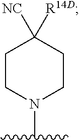

—R^{14D} is phenyl substituted with one group —R^{X14D};
—R¹⁶ is —H; —R¹⁷ is —H; and —R¹⁸ is —H.

(221) The compound according to (130), or a salt, hydrate, or solvate thereof, wherein:
—R¹⁴ is

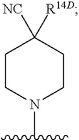

—R^{14D} is phenyl substituted with one group —R^{X14D};
-L¹¹- is —S(O)₂—; —R^{1L} is selected from C₁₋₆ alkyl; and
—R¹⁶ is —H; —R¹⁷ is —H; and —R¹⁸ is —H.

(222) The compound according to (130), or a salt, hydrate, or solvate thereof, wherein:
—R¹⁴ is

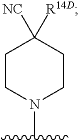

—R^{14D} is phenyl substituted with one group —R^{X14D};
-L¹¹- is —S(O)₂—; —R^{1L} is selected from C₁₋₆ alkyl; and
—R¹⁶ is —H; —R¹⁷ is —H; —R¹⁸ is —H; and
—R^{X14D} is a leaving group selected from boronic acid esters, and boronic acid.

(223) The compound according to (130), or a salt, hydrate, or solvate thereof, wherein:
—R¹⁴ is

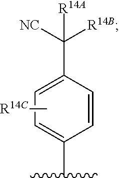

and
—R¹⁶ is a leaving group, for example —R¹⁶ is —R^{X16}.

(224) The compound according to (130), or a salt, hydrate, or solvate thereof, wherein:
—R¹⁴ is

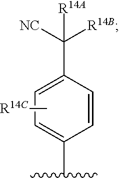

—R^{14A} and —R^{14B} together with the carbon atom to which they are attached form a C₃₋₅ cycloalkyl;
—R^{14C} is —H; and
—R¹⁶ is a leaving group, for example —R¹⁶ is —R^{X16}.

(225) The compound according to (130), or a salt, hydrate, or solvate thereof, wherein:
—R¹⁴ is

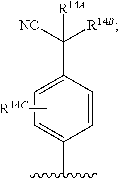

—R^{14A} and —R^{14B} together with the carbon atom to which they are attached form a C₃₋₅ cycloalkyl;
—R^{14C} is —H;
-L¹¹- is —S(O)₂—; —R^{1L} is selected from C₁₋₆ alkyl; and
—R¹⁶ is a leaving group, for example —R¹⁶ is —R^{X16}.

(226) The compound according to (130), or a salt, hydrate, or solvate thereof, wherein:
—RN is

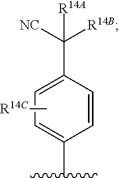

—R^{14A} and —R^{14B} together with the carbon atom to which they are attached form a C₃₋₅ cycloalkyl;
—R^{14C} is —H;
-L¹¹- is —S(O)₂—; —R^{1L} is selected from C₁₋₆ alkyl;
—R¹⁷ is —H; —R¹⁸ is —H; and
—R¹⁶ is a leaving group, for example —R¹⁶ is —R^{X16}.

(227) The compound according to (130), or a salt, hydrate, or solvate thereof, wherein:
—R¹⁴ is

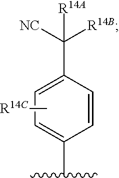

—R^{14A} and —R^{14B} together with the carbon atom to which they are attached form a C₃₋₅ cycloalkyl;
—R^{14C} is —H;
-L¹¹- is —S(O)₂—; —R^{1L} is selected from C₁₋₆ alkyl;
—R¹⁷ is —H; —R¹⁸ is —H;
—R¹⁶ is a leaving group, for example —R¹⁶ is —R^{X16}; and
the leaving group is selected from boronic acid esters, boronic acid, trialkylamines, organostannanes, aryliodonium salts, and arylsulfonium salts.

(228) The compound according to (130), or a salt, hydrate, or solvate thereof, wherein:
—R¹⁴ is

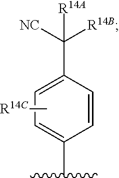

—R^{14A} and —R^{14B} together with the carbon atom to which they are attached form a C₃₋₅ cycloalkyl;
—R^{14C} is —H;
-L¹¹- is —S(O)₂—; —R^{1L} is selected from C₁₋₆ alkyl;
—R¹⁷ is —H; —R¹⁸ is —H;
—R¹⁶ is a leaving group, for example —R¹⁶ is —R^{X16}; and
the leaving group is selected from —BPin, —B(OH)₂, -[PhI]⁺BF₄⁻, -[Ph₂S]⁺TfO⁻, —SnMe₃, and —N⁺Me₃.

### Specific Compounds

(229) A compound according to (130), selected from compounds of the following formulae and salts, hydrates, and solvates thereof:

| Cmpd No. | Structure | Name |
|---|---|---|
| PQM-P-001 | 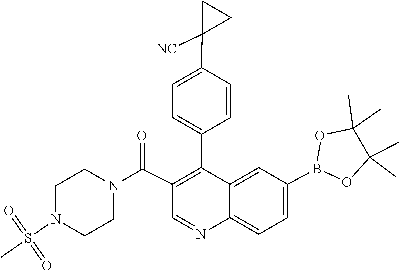 | 1-[4-[3-(4- methylsulfonylpiperazine-1- carbonyl)-6-(4,4,5,5- tetramethyl-1,3,2- dioxaborolan-2-yl)-4- quinolyl]phenyl]cyclo- propanecarbonitrile |
| PQM-P-002 | 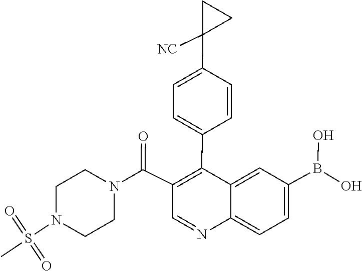 [SMILES: CS(=O)(=O)N1CCN(C(=O)c2cnc3ccc(B(O)O)cc3c2-c2ccc(C3(C#N)CC3)cc2)CC1] | [4-[4-(1- cyanocyclopropyl)phenyl]-3- (4-methylsulfonylpiperazine- 1-carbonyl)-6- quinolyl]boronic acid |
| PQM-P-003 | 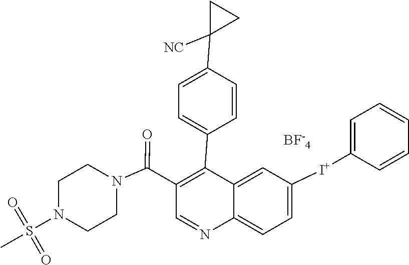 [SMILES: CS(=O)(=O)N1CCN(C(=O)c2cnc3ccc([I+]c4ccccc4)cc3c2-c2ccc(C3(C#N)CC3)cc2)CC1 ; "BF-4"] | [4-[4-(1- cyanocyclopropyl)phenyl]-3- (4-methylsulfonylpiperazine- 1-carbonyl)-6-quinolyl]- phenyl-iodonium tetrafluoroborate |
| PQM-P-004 | 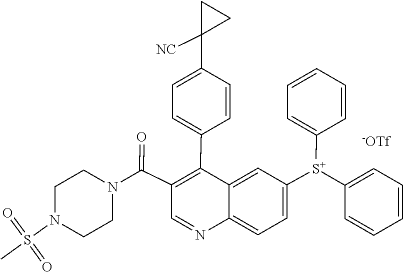 [SMILES: CS(=O)(=O)N1CCN(C(=O)c2cnc3ccc([S+](c4ccccc4)c4ccccc4)cc3c2-c2ccc(C3(C#N)CC3)cc2)CC1 ; "-OTf"] | [4-[4-(1- cyanocyclopropyl)phenyl]-3- (4-methylsulfonylpiperazine- 1-carbonyl )-6-quinolyl]- diphenyl-sulfonium trifluoromethanesulfonate |
| PQM-P-005 | 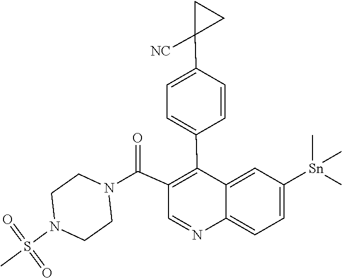 | 1-[4-[3-(4- methylsulfonylpiperazine-1- carbonyl)-6- trimethylstannyl-4- quinolyl]phenyl]cyclo- propanecarbonitrile |
| PQM-P-006 | 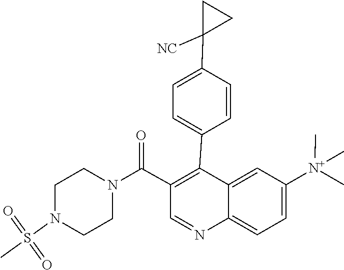 | [4-[4-(1- cyanocyclopropyl)phenyl]-3- (4-methylsulfonylpiperazine- 1-carbonyl)-6-quinolyl]- trimethyl-ammonium salt |
| PQM-P-007 | 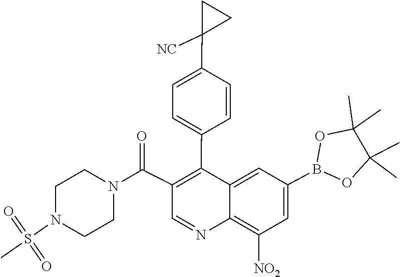 | 1-[4-[3-(4- methylsulfonylpiperazine-1- carbonyl)-8-nitro-6-(4,4,5,5- tetramethyl-1,3,2- dioxaborolan-2-yl)-4- quinolyl]phenyl]cyclo- propanecarbonitrile |
| PQM-P-008 | 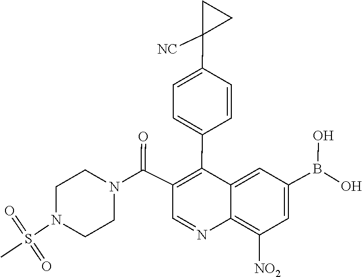 [SMILES: CS(=O)(=O)N1CCN(C(=O)c2cnc3c([N+](=O)[O-])cc(B(O)O)cc3c2-c2ccc(C3(C#N)CC3)cc2)CC1] | [4-[4-(1- cyanocyclopropyl)phenyl]-3- (4-methylsulfonylpiperazine- 1-carbonyl)-8-nitro-6- quinolyl]boronic acid |
| PQM-P-009 | 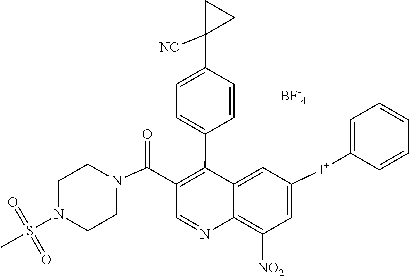 [SMILES: CS(=O)(=O)N1CCN(C(=O)c2cnc3c([N+](=O)[O-])cc([I+]c4ccccc4)cc3c2-c2ccc(C3(C#N)CC3)cc2)CC1 ; "BF-4"] | [4-[4-(1- cyanocyclopropyl)phenyl]-3- (4-methylsulfonylpiperazine- 1-carbonyl)-8-nitro-6- quinolyl]-phenyl-iodonium tetrafluoroborate |
| PQM-P-010 | 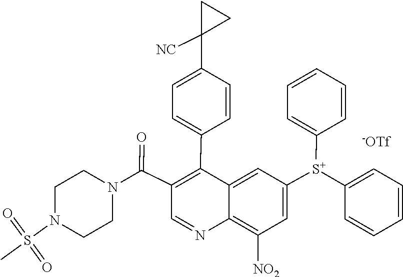 [SMILES: CS(=O)(=O)N1CCN(C(=O)c2cnc3c([N+](=O)[O-])cc([S+](c4ccccc4)c4ccccc4)cc3c2-c2ccc(C3(C#N)CC3)cc2)CC1 ; "-OTf"] | [4-[4-(1- cyanocyclopropyl)phenyl]-3- (4-methylsulfonylpiperazine- 1-carbonyl)-8-nitro-6- quinolyl]-diphenyl-sulfonium trifluoromethanesulfonate |
| PQM-P-011 | 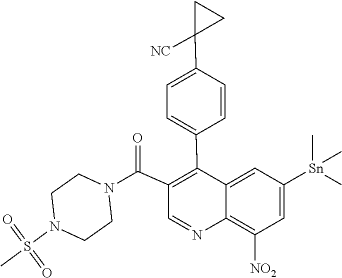 | 1-[4-[3-(4- methylsulfonylpiperazine-1- carbonyl)-8-nitro-6- trimethylstannyl-4- quinolyl]phenyl]cyclo- propanecarbonitrile |
| PQM-P-012 | 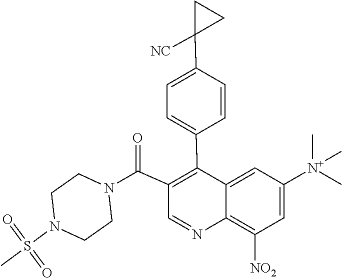 | [4-[4-(1- cyanocyclopropyl)phenyl]-3- (4-methylsulfonylpiperazine- 1-carbonyl)-8-nitro-6- quinolyl]-trimethyl- ammonium salt |
| PQM-P-013 | 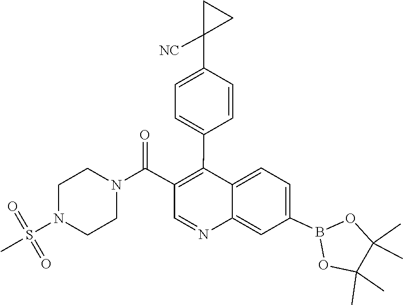 | 1-[4-[3-(4- methylsulfonylpiperazine-1- carbonyl)-7-(4,4,5,5- tetramethyl-1,3,2- dioxaborolan-2-yl)-4- quinolyl]phenyl]cyclo- propanecarbonitrile |
| PQM-P-014 | 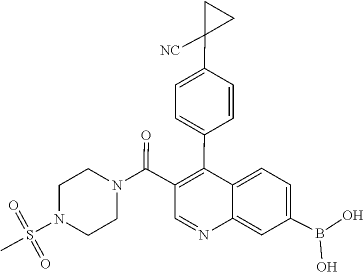 [SMILES: CS(=O)(=O)N1CCN(C(=O)c2cnc3cc(B(O)O)ccc3c2-c2ccc(C3(C#N)CC3)cc2)CC1] | [4-[4-(1- cyanocyclopropyl)phenyl]-3- (4-methylsulfonylpiperazine- 1-carbonyl)-7- quinolyl]boronic acid |
| PQM-P-015 | 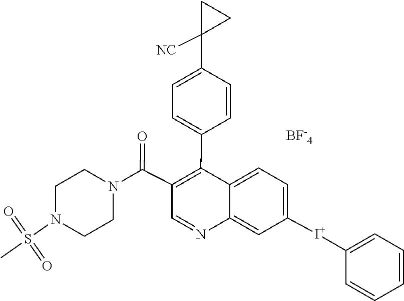 [SMILES: CS(=O)(=O)N1CCN(C(=O)c2cnc3cc([I+]c4ccccc4)ccc3c2-c2ccc(C3(C#N)CC3)cc2)CC1 ; "BF-4"] | [4-[4-(1- cyanocyclopropyl)phenyl]-3- (4-methylsulfonylpiperazine- 1-carbonyl)-7-quinolyl]- phenyl-iodonium tetrafluoroborate |
| PQM-P-016 | 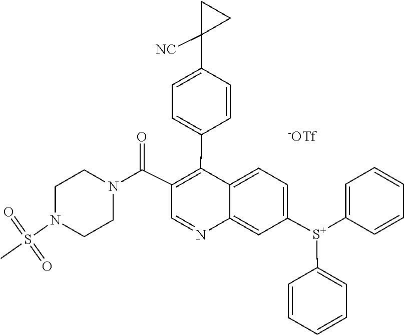 [SMILES: CS(=O)(=O)N1CCN(C(=O)c2cnc3cc([S+](c4ccccc4)c4ccccc4)ccc3c2-c2ccc(C3(C#N)CC3)cc2)CC1 ; "-OTf"] | [4-[4-(1- cyanocyclopropyl)phenyl]-3- (4-methylsulfonylpiperazine- 1-carbonyl)-7-quinolyl]- diphenyl-sulfonium trifluoromethanesulfonate |
| PQM-P-017 | 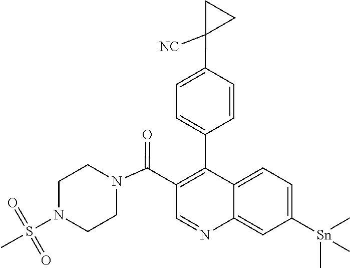 | 1-[4-[3-(4- methylsulfonylpiperazine-1- carbonyl)-7- trimethylstannyl-4- quinolyl]phenyl]cyclo- propanecarbonitrile |
| PQM-P-018 | 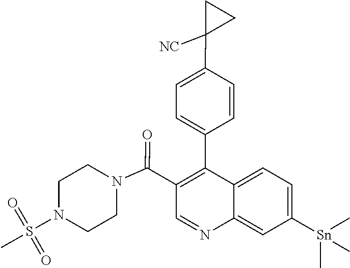 | [4-[4-(1- cyanocyclopropyl)phenyl]-3- (4-methylsulfonylpiperazine- 1-carbonyl)-7-quinolyl]- trimethyl-ammonium salt |
| PQM-P-019 | 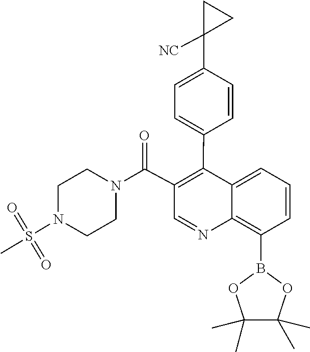 | 1-[4-[3-(4- methylsulfonylpiperazine-1- carbonyl)-8-(4,4,5,5- tetramethyl-1,3,2- dioxaborolan-2-yl)-4- quinolyl]phenyl]cyclo- propanecarbonitrile |
| PQM-P-020 | 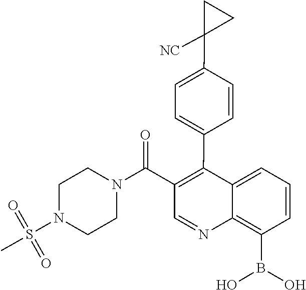 [SMILES: CS(=O)(=O)N1CCN(C(=O)c2cnc3c(B(O)O)cccc3c2-c2ccc(C3(C#N)CC3)cc2)CC1] | [4-[4-(1- cyanocyclopropyl)phenyl]-3- (4-methylsulfonylpiperazine- 1-carbonyl)-8- quinolyl]boronic acid |
| PQM-P-021 | 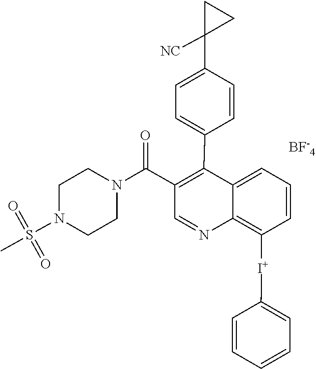 [SMILES: CS(=O)(=O)N1CCN(C(=O)c2cnc3c([I+]c4ccccc4)cccc3c2-c2ccc(C3(C#N)CC3)cc2)CC1 ; "BF-4"] | [4-[4-(1- cyanocyclopropyl)phenyl]-3- (4-methylsulfonylpiperazine- 1-carbonyl)-8-quinolyl]- phenyl-iodonium tetrafluoroborate |
| PQM-P-022 | 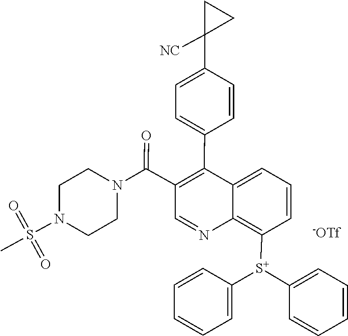 [SMILES: CS(=O)(=O)N1CCN(C(=O)c2cnc3c([S+](c4ccccc4)c4ccccc4)cccc3c2-c2ccc(C3(C#N)CC3)cc2)CC1 ; "-OTf"] | [4-[4-(1- cyanocyclopropyl)phenyl]-3- (4-methylsulfonylpiperazine- 1-carbonyl)-8-quinolyl]- diphenyl-sulfonium trifluoromethanesulfonate |
| PQM-P-023 | 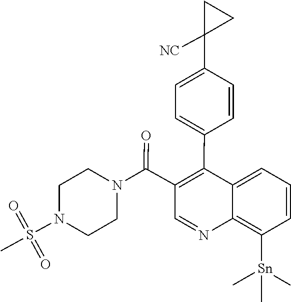 | 1-[4-[3-(4- methylsulfonylpiperazine-1- carbonyl)-8- trimethylstannyl-4- quinolyl]phenyl]cyclo- propanecarbonitrile |
| PQM-P-024 | 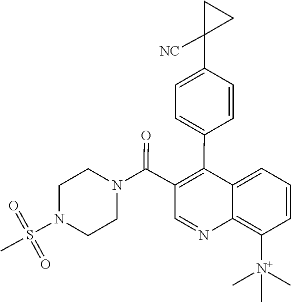 | [4-[4-(1- cyanocyclopropyl)phenyl]-3- (4-methylsulfonylpiperazine- 1-carbonyl)-8-quinolyl]- trimethyl-ammonium salt |
| PQM-P-025 | 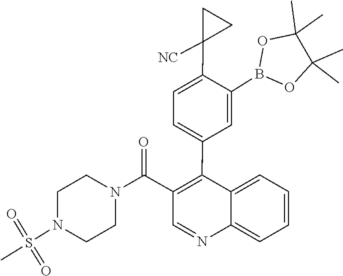 | 1-[4-[3-(4- methylsulfonylpiperazine-1- carbonyl)-4-quinolyl]-2- (4,4,5,5-tetramethyl-1,3,2- dioxaborolan-2- yl)phenyl]cyclo- propanecarbonitrile |
| PQM-P-026 | 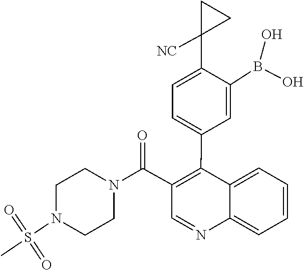 [SMILES: CS(=O)(=O)N1CCN(C(=O)c2cnc3ccccc3c2-c2ccc(C3(C#N)CC3)c(B(O)O)c2)CC1] | [2-(1-cyanocyclopropyl)-5- [3-(4- methylsulfonylpiperazine-1- carbonyl)-4- quinolyl]phenyl]boronic acid |
| PQM-P-027 | 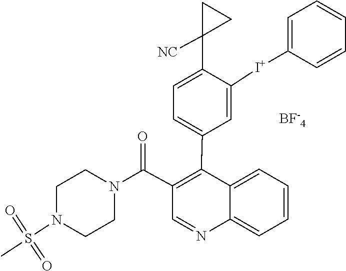 | [2-(1-cyanocyclopropyl)-5- [3-(4- methylsulfonylpiperazine-1- carbonyl)-4-quinolyl]phenyl]- phenyl-iodonium tetrafluoroborate |
| PQM-P-028 | 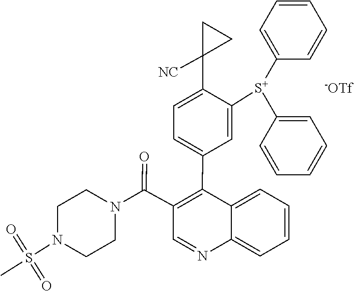 [SMILES: CS(=O)(=O)N1CCN(C(=O)c2cnc3ccccc3c2-c2ccc(C3(C#N)CC3)c([S+](c3ccccc3)c3ccccc3)c2)CC1 ; "-OTf"] | [2-(1-cyanocyclopropyl)-5- [3-(4- methylsulfonylpiperazine-1- carbonyl)-4-quinolyl]phenyl]- diphenyl-sulfonium trifluoromethanesulfonate |
| PQM-P-029 | 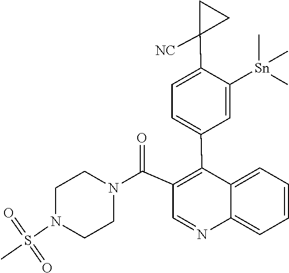 [SMILES: CS(=O)(=O)N1CCN(C(=O)c2cnc3ccccc3c2-c2ccc(C3(C#N)CC3)[c]([Sn]([CH3])([CH3])[CH3])c2)CC1] | 1-[4-[3-(4- methylsulfonylpiperazine-1- carbonyl)-4-quinolyl]-2- trimethylstannyl- phenyl]cyclopropane- carbonitrile |
| PQM-P-030 | 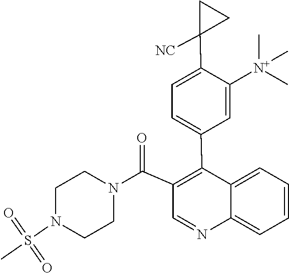 [SMILES: C[N+](C)(C)c1cc(-c2c(C(=O)N3CCN(S(C)(=O)=O)CC3)cnc3ccccc23)ccc1C1(C#N)CC1] | [2-(1-cyanocyclopropyl)-5- [3-(4- methylsulfonylpiperazine-1- carbonyl)-4-quinolyl]phenyl]- trimethyl-ammonium salt |
| PQM-P-031 | 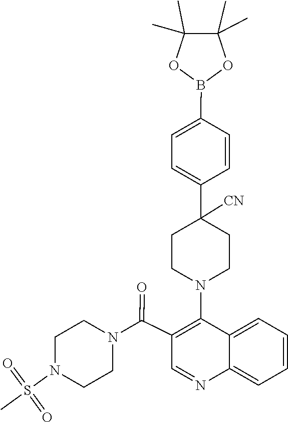 [SMILES: CC1(C)OB(c2ccc(C3(C#N)CCN(c4c(C(=O)N5CCN(S(C)(=O)=O)CC5)cnc5ccccc45)CC3)cc2)OC1(C)C] | 1-[3-(4- methylsulfonylpiperazine-1- carbonyl)-4-quinolyl]-4-[4- (4,4,5,5-tetramethyl-1,3,2- dioxaborolan-2- yl)phenyl]piperidine-4- carbonitrile |
| PQM-P-032 | 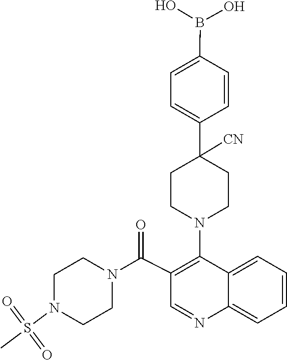 [SMILES: CS(=O)(=O)N1CCN(C(=O)c2cnc3ccccc3c2N2CCC(C#N)(c3ccc(B(O)O)cc3)CC2)CC1] | [4-[4-cyano-1-[3-(4- methylsulfonylpiperazine-1- carbonyl)-4-quinolyl]-4- piperidyl]phenyl]boronic acid |
| PQM-P-033 | 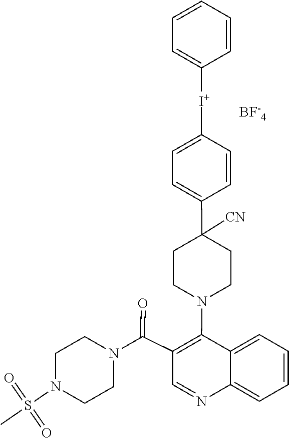 | [4-[4-cyano-1-[3-(4- methylsulfonylpiperazine-1- carbonyl)-4-quinolyl]-4- piperidyl]phenyl]-phenyl- iodonium tetrafluoroborate |
| PQM-P-034 | 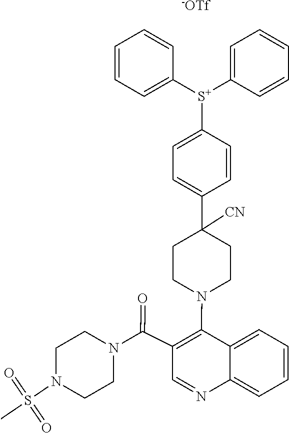 [SMILES: CS(=O)(=O)N1CCN(C(=O)c2cnc3ccccc3c2N2CCC(C#N)(c3ccc([S+](c4ccccc4)c4ccccc4)cc3)CC2)CC1 ; "-OTf"] | [4-[4-cyano-1-[3-(4- methylsulfonylpiperazine-1- carbonyl)-4-quinolyl]-4- piperidyl]phenyl]-diphenyl- sulfonium trifluoromethanesulfonate |
| PQM-P-035 | 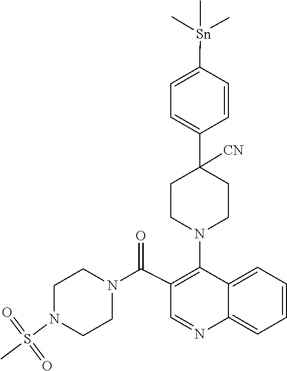 [SMILES: CS(=O)(=O)N1CCN(C(=O)c2cnc3ccccc3c2N2CCC(C#N)(c3cc[c]([Sn]([CH3])([CH3])[CH3])cc3)CC2)CC1] | 1-[3-(4- methylsulfonylpiperazine-1- carbonyl)-4-quinolyl]-4-(4- trimethylstannylphenyl) piperidine-4-carbonitrile |
| PQM-P-036 | 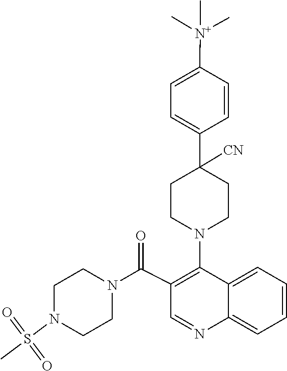 [SMILES: C[N+](C)(C)c1ccc(C2(C#N)CCN(c3c(C(=O)N4CCN(S(C)(=O)=O)CC4)cnc4ccccc34)CC2)cc1] | [4-[4-cyano-1-[3-(4- methylsulfonylpiperazine-1- carbonyl)-4-quinolyl]-4- piperidyl]phenyl]-trimethyl- ammonium salt |

(230) A compound according to (130), selected from compounds of the following formulae and salts, hydrates and solvates thereof:

| Cmpd No. | Structure | Name |
|---|---|---|
| PQM-P-001 | 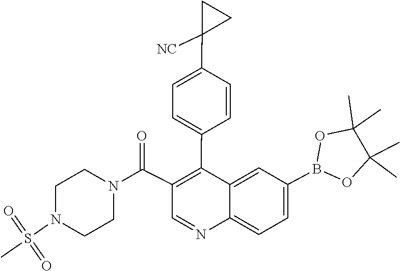 | 1-[4-[3-(4- methylsulfonylpiperazine- 1-carbonyl)-6-(4,4,5,5- tetramethyl-1,3,2- dioxaborolan-2-yl)-4- quinolyl]phenyl]cyclo- propanecarbonitrile |
| PQM-P-007 | 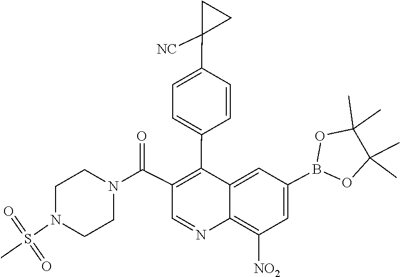 | 1-[4-[3-(4- methylsulfonylpiperazine- 1-carbonyl)-8-nitro-6- (4,4,5,5-tetramethyl-1,3,2- dioxaborolan-2-yl)-4- quinolyl]phenyl]cyclo- propanecarbonitrile |
| PQM-P-013 | 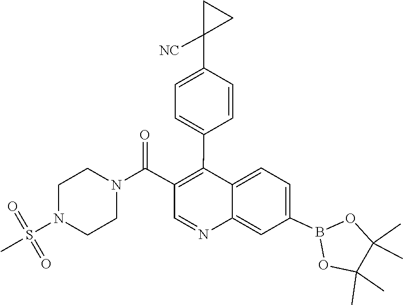 | 1-[4-[3-(4- methylsulfonylpiperazine- 1-carbonyl)-7-(4,4,5,5- tetramethyl-1,3,2- dioxaborolan-2-yl)-4- quinolyl]phenyl]cyclo- propanecarbonitrile |
| PQM-P-020 | 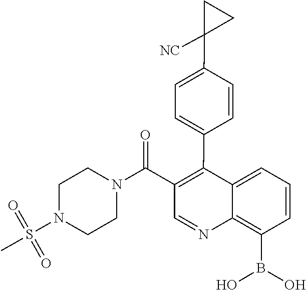 [SMILES: CS(=O)(=O)N1CCN(C(=O)c2cnc3c(B(O)O)cccc3c2-c2ccc(C3(C#N)CC3)cc2)CC1] | [4-[4-(1- cyanocyclopropyl)phenyl]- 3-(4- methylsulfonylpiperazine- 1-carbonyl)-8- quinolyl]boronic acid |
| PQM-P-025 | 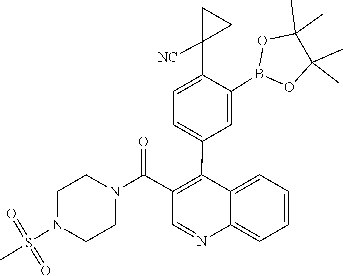 | 1-[4-[3-(4- methylsulfonylpiperazine- 1-carbonyl)-4-quinolyl]-2- (4,4,5,5-tetramethyl-1,3,2- dioxaborolan-2- yl)phenyl]cyclo- propanecarbonitrile |
| PQM-P-031 | 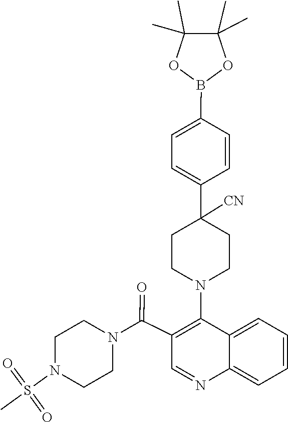 [SMILES: CC1(C)OB(c2ccc(C3(C#N)CCN(c4c(C(=O)N5CCN(S(C)(=O)=O)CC5)cnc5ccccc45)CC3)cc2)OC1(C)C] | 1-[3-(4- methylsulfonylpiperazine- 1-carbonyl)-4-quinolyl]-4- [4-(4,4,5,5-tetramethyl- 1,3,2-dioxaborolan-2- yl)phenyl]piperidine-4- carbonitrile |

(231) A compound according to (130), that is:

| Cmpd No. | Structure | Name |
|---|---|---|
| PQM-P-001 | 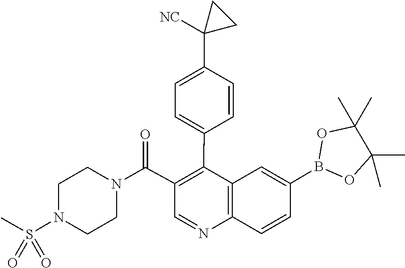 | 1-[4-[3-(4- methylsulfonylpiperazine-1- carbonyl)-6-(4,4,5,5- tetramethyl-1,3,2- dioxaborolan-2-yl)-4- quinolyl]phenyl]- cyclopropanecarbonitrile |

(232) A compound according to (130), that is 1-[4-[3-(4-methylsulfonylpiperazine-1-carbonyl)-8-nitro-6-(4,4,5,5-tetramethyl-1,3,2-dioxaborolan-2-yl)-4-quinolyl]phenyl]cyclopropanecarbonitrile (PQM-P-007).

(233) A compound according to (130), that is [4-[4-(1-cyanocyclopropyl)phenyl]-3-(4-methylsulfonylpiperazine-1-carbonyl)-8-quinolyl]boronic acid (PQM-P-020).

(234) A compound according to (130), that is 1-[3-(4-methylsulfonylpiperazine-1-carbonyl)-4-quinolyl]-4-[4-(4,4,5,5-tetramethyl-1,3,2-dioxaborolan-2-yl)phenyl]piperidine-4-carbonitrile (PQM-P-031).

For the avoidance of doubt:

The index “C_{x-y}” in terms such as “C₉₋₁₀heteroaryl”, “C₃₋₇heterocyclyl”, and the like, refers to the number of ring atoms, which may be carbon atoms or heteroatoms (e.g., N, O, S, as the case may be). For example, pyridyl is an example of a C₆heteroaryl group, and piperidino is an example of a C₆heterocyclyl group.

Unless otherwise indicated, where a compound is shown or described which has one or more chiral centres, and two or more stereoisomers are possible, all such stereoisomers are disclosed and encompassed, both individually (e.g., as isolated from the other stereoisomer(s)) and as mixtures (e.g., as equimolar or non-equimolar mixtures of two or more stereoisomers). For example, unless otherwise indicated, where a compound has one chiral centre, each of the (R) and (S) enantiomers are disclosed and encompassed, both individually (e.g., as isolated from the other enantiomer) and as a mixture (e.g., as equimolar or non-equimolar mixtures of the two enantiomers). For example, the initial carbon atom of a pendant sec-butyl group, —CH(CH₃)CH₂CH₃ is usually chiral, and so gives rise to stereoisomers, e.g., (R) and (S) enantiomers if it is the only chiral centre, each of which is disclosed and encompassed.

In certain cases, a subsequent embodiment may refer to an earlier embodiment and the subsequent embodiment recites a feature that is not permitted by the earlier embodiment. In such cases “if present” is used after the recited feature in the subsequent embodiment to denote that the definition of the subsequent embodiment only applies to the earlier embodiments in which that feature is permitted (i.e., only those earlier embodiments in which the same feature is present within the definition). For example, if one embodiment defines —R⁴ as the phenyl variant comprising —R^{4A}, —R^{4B}, and —R^{4C}, then the piperidinyl —R⁴ variant comprising —R^{4D} is not present in that embodiment. Accordingly, a subsequent embodiment which recites a definition for —R^{4D} (only present in the piperidinyl variant of —R⁴) but refers to the earlier embodiment which defines —R⁴ as the phenyl variant (in which —R^{4D} is not present) will use the terminology “if present” to indicate that the features of the subsequent embodiment are only applicable earlier embodiments in which that same feature is permitted (i.e., present in the definition of compounds).

Likewise, certain definitions recited in the embodiments may refer to a feature which is present in earlier embodiments, but define that feature in such a way that is not applicable in light of an earlier referred to embodiment (e.g., broader than or different to the definition used in the earlier embodiment). For example, in some embodiments, the group —R^{4C} may be —H or —F, in other embodiments, —R^{4C} may additionally be —R^{X4C} which is a radioisotope. This latter definition of —R^{4C} which includes —R^{X4C} is different to the earlier embodiment which only permits —R^{4C} to be —H or —F. In such cases, the phrase “as applicable” is used to indicate that the definition recited in that embodiment only applies to the earlier referred to embodiments in which that definition of the features (e.g., the definition of —R^{4C}) is permitted.

### Combinations

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the chemical groups represented by the variables (e.g., —R⁴, -L¹-, —R⁶, etc.) are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In this context, the skilled person will readily appreciate that certain combinations of groups (e.g., substituents) may give rise to compounds which may not be readily synthesized and/or are chemically unstable. In addition, all sub-combinations of the chemical groups listed in the embodiments describing such variables are also specifically embraced by the present invention and are disclosed herein just as if each and every such sub-combination of chemical groups was individually and explicitly disclosed herein.

### Substantially Purified Forms

One aspect of the present invention pertains to PQM-H or PQM-P compounds, as described herein, in substantially purified form and/or in a form substantially free from contaminants.

In one embodiment, the substantially purified form is at least 50% by weight of a desired compound with respect to other components, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless otherwise specified, the substantially purified form refers to the desired compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to a equimolar mixture of enantiomers (i.e., a racemic mixture, a racemate). In one embodiment, the substantially purified form refers to one enantiomer, e.g., optically pure enantiomer. In one embodiment, the substantially purified form refers to a compound containing a specific isotope, e.g., containing a given radioisotope as opposed to other atoms (e.g., non-radioactive isotopes or products of radioactive decay) at a given position.

In one embodiment, the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer. In one embodiment, the contaminants refer to compounds containing non-radioactive isotopes, products of decay, or non-desired radioactive isotopes.

In one embodiment, the substantially purified form is at least 60% optically pure (i.e., 60% of the compound, on a molar basis, is the desired stereoisomer or enantiomer, and 40% is the undesired stereoisomer or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

### Isomers

Certain compounds may exist in one or more particular geometric, optical, enantiomeric, diastereoisomeric, epimeric, atropic, stereoisomeric, tautomeric, conformational, or anomeric forms, including but not limited to, cis- and trans-forms; E- and Z-forms; c-, t-, and r- forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

A reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl). However, reference to a specific group or substitution pattern is not intended to include other structural (or constitutional isomers) which differ with respect to the connections between atoms rather than by positions in space. For example, a reference to a methoxy group, —OCH₃, is not to be construed as a reference to its structural isomer, a hydroxymethyl group, —CH₂OH. Similarly, a reference specifically to ortho-chlorophenyl is not to be construed as a reference to its structural isomer, meta-chlorophenyl.

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro. A reference herein to one tautomer is intended to encompass both tautomers.

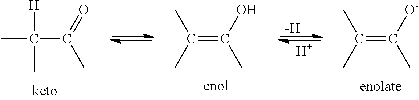

For example, 1H-pyridin-2-one-5-yl and 2-hydroxyl-pyridin-5-yl (shown below) are tautomers of one another. A reference herein to one is intended to encompass both.

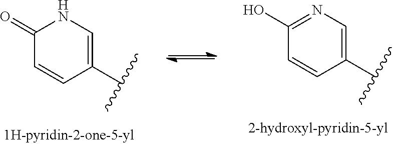 [SMILES: *c1ccc(=O)[nH]c1 ; *c1ccc(O)nc1]

Note that specifically included in the term “isomer” are compounds with one or more isotopic substitutions where the atomic mass of a specific atom is not already given. For example, —H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like. Note, however, that where the atomic mass of a given atom is defined, e.g., in the case of a radioisotope, then the structural formula does not contemplate isotopic replacements at that position. For example, when a position is defined as being ¹⁸F, then it is not contemplated that that fluorine atom may be replaced with other isotopic forms such as ¹⁹F.

In this context, as used herein, when a substituent position is defined as being “a radioisotope”, then that means that the substituent at that position is enriched in a radioisotope, for example, ¹⁸F, ¹²³I, ¹²⁴I, ¹²⁵I, ¹³¹I, or ²¹¹At, such that an amount of the compound comprises the radioisotope at an abundance that is higher than the natural abundance of said radioisotope.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the compound, for example, a pharmaceutically-acceptable salt. Examples of pharmaceutically acceptable salts are discussed in Berge et al, 1977, “Pharmaceutically Acceptable Salts,” J. Pharm. Sci., Vol. 66, pp. 1-19.

For example, if the compound is anionic, or has a functional group, which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺ as well as the ammonium ion (i.e., NH₄⁺). Examples of suitable organic cations include, but are not limited to substituted ammonium ions (e.g., NH₃R⁺, NH₂R₂⁺, NHR₃⁺, NR₄⁺), for example, where each R is independently linear or branched saturated C₁₋₁₈alkyl, C₃₋₈cycloalkyl, C₃₋₈cycloalkyl-C₁₋₆alkyl, and phenyl-C₁₋₆alkyl, wherein the phenyl group is optionally substituted. Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group, which upon protonation may become cationic (e.g., —NH₂ may become —NH₃⁺), then a salt may be formed with a suitable anion.

For example, if a parent structure contains a cationic group (e.g., —NMe₂⁺), or has a functional group, which upon protonation may become cationic (e.g., —NH₂ may become —NH₃⁺), then a salt may be formed with a suitable anion. In the case of a quaternary ammonium compound a counter-anion is generally always present in order to balance the positive charge. If, in addition to a cationic group (e.g., —NMe₂⁺, —NH₃⁺), the compound also contains a group capable of forming an anion (e.g., —COOH), then an inner salt (also referred to as a zwitterion) may be formed.

Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyloxybenzoic, acetic, trifluoroacetic, ascorbic, aspartic, benzoic, camphorsulfonic, cinnamic, citric, edetic, 1,2-ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Examples of suitable counter-ions which are especially suitable for quaternary ammonium compounds (e.g., those with a —NMe₂⁺ group) include 1-adamantanesulfonate, benzenesulfonate, bisulfate, bromide, chloride, iodide, methanesulfonate, methylsulfate, 1,5-napthalene-bis-sulfonate, 4-nitrobenzenesulfonate, formate, tartrate, tosylate, trifluoroacetate, trifluoromethylsulfonate, sulphate. Again, if the compound also contains a group capable of forming an anion (e.g., —COOH), then an inner salt may be formed.

Unless otherwise specified, a reference to a particular compound also includes salt forms thereof.

### Solvates and Hydrates

It may be convenient or desirable to prepare, purify, and/or handle a corresponding solvate of the compound. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate and hydrate forms thereof.

### Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well-known chemical methods are employed to reversibly render unreactive a functional group, which otherwise would be reactive, under specified conditions. In a chemically protected form, one or more reactive functional groups are in the form of a protected or protecting group (alternatively as a masked or masking group or a blocked or blocking group). By protecting a reactive functional group, reactions involving other unprotected reactive functional groups can be performed, without affecting the protected group; the protecting group may be removed or the masking group transformed, usually in a subsequent step, without substantially affecting the remainder of the molecule. See, for example, Protective Groups in Organic Synthesis (T. Green and P. Wuts; 4th Edition; John Wiley and Sons, 2006).

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a 1,1-diether (>C(OR)₂), by reaction with, for example, a primary alcohol in the presence of an acid. The aldehyde or ketone group is readily regenerated, for example, by hydrolysis using water in the presence of acid.

For example, an amine group may be protected, for example, as an amide (—NRCO—R) or a urethane (—NRCO—OR), for example, as: an acetamide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulfonyl)ethyloxy amide (—NH-Psec); or, in suitable cases (e.g., cyclic amines), as a nitroxide radical (>N−O•).

For example, a carboxylic acid group may be protected as an ester for example, as: an C₁₋₇alkyl ester (e.g., a methyl ester; a t-butyl ester); a C₁₋₇haloalkyl ester (e.g., a 2,2,2-trihaloethyl ester); a 2-tri(C₁₋₇alkyl)silyl-ethyl ester; or a C₅₋₂₀aryl-C₁₋₇alkyl ester (e.g., a benzyl ester; a nitrobenzyl ester); or as an amide or hydrazide, for example, as acetamide or a N,N,N′-trimethylhydrazide.

For example, a thiol group may be protected as a thioether (—SR), for example, as: a benzyl thioether; an acetamidomethyl ether (—S—CH₂NHC(═O)CH₃).

### Prodrugs

It may be convenient or desirable to prepare, purify, and/or handle the compound in the form of a prodrug. The term “prodrug,” as used herein, pertains to a compound, which yields the desired active compound in vivo. Typically, the prodrug is inactive, or less active than the desired active compound, but may provide advantageous handling, administration, or metabolic properties.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the active compound, or a compound, which, upon further chemical reaction, yields the active compound (for example, as in antibody directed enzyme prodrug therapy (ADEPT), gene directed enzyme prodrug therapy (GDEPT), lipid directed enzyme prodrug therapy (LIDEPT), etc.). For example, the prodrug may be a sugar derivative or other glycoside conjugate, or may be an amino acid ester derivative.

### Compositions

One aspect of the present invention pertains to a composition (e.g., a pharmaceutical composition) comprising a PQM-H compound, as described herein, and a pharmaceutically acceptable carrier, diluent, or excipient.

Another aspect of the present invention pertains to a method of preparing a composition (e.g., a pharmaceutical composition) comprising mixing a PQM-H compound, as described herein, and a pharmaceutically acceptable carrier, diluent, or excipient.

### Uses

The PQM-H compounds described herein are useful in methods of molecular imaging of cells expressing ALDH1A1, for example in methods of molecular medical imaging. In particular, the PQM-H compounds are suitable for imaging of cells expressing ALDH1A1 by PET, SPECT, and/or scintigraphy.

The PQM-H compounds described herein are useful in the treatment of, for example, cancer (as “anti-cancer agents”, in particular for radiotherapy) etc.

The PQM-P compounds described herein are useful in methods of producing PQM-H compounds.

### Methods of Medical Imaging

Another aspect of the present invention pertains to a method of medical imaging (e.g., a method of imaging a population of cells expressing ALDH1A1) comprising administering (i.e., delivering) a PQM-H compound, as described herein, to a subject and then imaging the subject by PET, SPECT, or scintigraphy.

The methods of medical imaging described herein are methods of molecular imaging.

In one embodiment, the subject is imaged by PET.

In one embodiment, the subject is imaged by SPECT.

In one embodiment, the subject is imaged by scintigraphy.

PET, SPECT, and scintigraphy each rely on the detection and counting of gamma photons. Without wishing to be bound by theory, in some embodiments, the PQM-H compounds described herein, once administered to a subject, accumulate at a target location, e.g., a population of cells expressing ALDH1A1 (such as a population of cancerous cells) and give rise to emission of gamma photons from that location (e.g., by positron emission followed by annihilation, by gamma decay, as a result of electron capture, or through subsequent decay of a daughter isotope). The target cell populations expressing ALDH1A1, at which the PQM-H compound has accumulated, can then be detected by collection of the emitted gamma photons, e.g., in through imaging the subject by PET, SPECT, or scintigraphy.

In one embodiment, the method of medical imaging further comprises the step of instilling the radioisotope on the PQM-H compound, for example, by suitable reaction with a corresponding PQM-P compound.

### Use in Methods of Diagnosis

Another aspect of the present invention pertains to a PQM-H compound, as described herein, for use in a method of diagnosis of a disorder (e.g., a disease) of the human or animal body, for example, for use in a method of diagnosis of a disorder (e.g., a disease) as described herein.

### Methods of Diagnosis

Another aspect of the present invention pertains to a method of diagnosis, for example, a method of diagnosis of a disorder (e.g., a disease) as described herein, comprising administering to a subject an effective amount of a PQM-H compound, as described herein, preferably in the form of a pharmaceutical composition.

In one embodiment, the method of diagnosis comprises performing on the subject, a method of medical imaging as described herein.

In one embodiment, the subject is imaged by PET.

In one embodiment, the subject is imaged by SPECT.

In one embodiment, the subject is imaged by scintigraphy.

In one embodiment, the method of diagnosis further comprises inspection of the image generated by the method of medical imaging and identification of locations at which the PQM-H compound has accumulated.

In one embodiment, the method of diagnosis further comprises the step of processing the image and comparison with standard values to determine the level of accumulation of PQM-H compound at the identified location.

In one embodiment, the method further comprises the step of attributing differences in the level of accumulation of PQM-H compound to a disorder (e.g., a disease, as described herein). In one embodiment, increased accumulation of PQM-H compound at a location is attributed to a population of cancer cells.

In one embodiment, the population of cancer cells are treatment resistant cancer cells.

### Use in Methods of Therapy

Another aspect of the present invention pertains to a PQM-H compound, as described herein, for use in a method of treatment of the human or animal body by therapy, for example, for use a method of treatment of a disorder (e.g., a disease) as described herein.

### Use in the Manufacture of Medicaments

Another aspect of the present invention pertains to use of a PQM-H compound, as described herein, in the manufacture of a medicament, for example, for use in a method of treatment, for example, for use a method of treatment of a disorder (e.g., a disease) as described herein.

In one embodiment, the medicament comprises the PQM-H compound.

### Methods of Treatment

Another aspect of the present invention pertains to a method of treatment, for example, a method of treatment of a disorder (e.g., a disease) as described herein, comprising administering to a subject in need of treatment a therapeutically-effective amount of a PQM-H compound, as described herein, preferably in the form of a pharmaceutical composition.

Disorders Associated with ALDH

In one embodiment (e.g., for use in methods of diagnosis, of methods of diagnosis, for use in methods of therapy, of use in the manufacture of medicaments, of methods of treatment), the disorder is: a disorder (e.g., a disease) that is associated with ALDH, especially ALDH1A1; a disorder (e.g., a disease) resulting from an inappropriate activity of a ALDH, especially ALDH1A1; a disorder (e.g., a disease) that is associated with ALDH mutation, especially ALDH1A1 mutation; a disorder (e.g., a disease) that is associated with ALDH overexpression, especially ALDH1A1 overexpression; a disorder (e.g., a disease) that is ameliorated by the inhibition (e.g., selective inhibition) of ALDH, especially ALDH1A1.

In one embodiment (e.g., for use in methods of diagnosis, of methods of diagnosis, for use in methods of therapy, of use in the manufacture of medicaments, of methods of treatment), the disorder is a disorder (e.g., a disease) that is associated with ALDH, especially ALDH1A1.

In one embodiment, the disorder is a disorder (e.g., a disease) resulting from an inappropriate activity of ALDH, especially ALDH1A1.

In one embodiment, the disorder is a disorder (e.g., a disease) that is associated with ALDH mutation, especially ALDH1A1 mutation; ALDH overexpression, especially ALDH1A1 overexpression (e.g., as compared to corresponding normal cells; e.g., wherein the overexpression is by a factor of 1.5, 2, 3, 5, 10, 20 or 50); or upstream pathway activation of ALDH, especially ALDH1A1.

In some embodiments, disorder is a disorder (e.g., a disease) that is associated with ALDH overexpression that is modulated through epigenetic regulation, especially ALDH1A1 overexpression that is modulated through epigenetic regulation.

In one embodiment, the disorder is a disorder (e.g., a disease) that is ameliorated by the inhibition (e.g., selective inhibition) of ALDH, especially ALDH1A1.

### Specific Disorders

In one embodiment (e.g., for use in methods of diagnosis, of methods of diagnosis, for use in methods of therapy, of use in the manufacture of medicaments, of methods of treatment), the disorder is cancer, for example, treatment resistant cancer.

In one embodiment (e.g., for use in methods of diagnosis, of methods of diagnosis, for use in methods of therapy, of use in the manufacture of medicaments, of methods of treatment), the disorder is: a treatment resistant disorder (e.g., a disease).

In one embodiment, the treatment resistant disorder is a disorder (e.g., a disease) in a patient who has received prior therapeutic treatments, but who receives little or no further clinical benefit from those treatments.

This includes, for example, patients who have received prior therapeutic treatments with standard-of-care treatments.

In one embodiment, the treatment resistant disorder is treatment resistant cancer.

In one embodiment, the treatment resistant cancer, is a cancer that does not respond (e.g., reduce in size, reduce in replication rate, enter remission, etc.) to clinically-approved therapies.

In one embodiment, the clinically-approved therapies are standard-of-care therapies.

In one embodiment, the standard-of-care therapy is a chemotherapy.

In one embodiment, the standard-of-care therapy is radiotherapy.

In one embodiment, the standard-of-care therapy is a targeted therapy.

In one embodiment, the chemotherapy includes treatment with a drug selected from:
cyclophosphamide, platinum-based treatment, gemcitabine, paclitaxel, etoposide, doxorubicin, 5-fluouricil, or combinations thereof.

In one embodiment, the chemotherapy includes treatment with a cyclophosphamide. This drug is directly detoxified by ALDH1A1.

In one embodiment, the targeted therapy is selected from PARP inhibitors, CDK inhibitors, and/or HER2 directed therapies

In one embodiment (e.g., for use in methods of diagnosis, of methods of diagnosis, for use in methods of therapy, of use in the manufacture of medicaments, of methods of treatment), the disorder is cancer.

In one embodiment, the cancer is treatment resistant cancer.

In one embodiment, the cancer is a cancer metastasis.

Included among cancers are:

(1) Carcinomas, including tumours derived from stratified squamous epithelia (squamous cell carcinomas) and tumours arising within organs or glands (adenocarcinomas). Examples include breast, colon, lung, prostate, ovary.

(2) Sarcomas, including: osteosarcoma and osteogenic sarcoma (bone); chondrosarcoma (cartilage); leiomyosarcoma (smooth muscle); rhabdomyosarcoma (skeletal muscle); mesothelial sarcoma and mesothelioma (membranous lining of body cavities); fibrosarcoma (fibrous tissue); angiosarcoma and haemangioendothelioma (blood vessels); liposarcoma (adipose tissue); glioma and astrocytoma (neurogenic connective tissue found in the brain); myxosarcoma (primitive embryonic connective tissue); mesenchymous and mixed mesodermal tumour (mixed connective tissue types).

(3) Myeloma.

(4) Haematopoietic tumours, including: myelogenous and granulocytic leukaemia (malignancy of the myeloid and granulocytic white blood cell series), e.g., chronic myeloid leukemia (CML), acute myeloid leukemia (AML); lymphatic, lymphocytic, and lymphoblastic leukaemia (malignancy of the lymphoid and lymphocytic blood cell series), e.g., acute lymphoblastic leukemia (ALL), chronic lymphocytic leukemia (CLL); polycythaemia vera (malignancy of various blood cell products, but with red cells predominating).

(5) Lymphomas, including: Hodgkin and Non-Hodgkin lymphomas.

(6) Mixed Types, including, e.g., adenosquamous carcinoma; mixed mesodermal tumour; carcinosarcoma; teratocarcinoma.

For example, in one embodiment, the disorder is breast cancer, ovarian cancer, prostate cancer, colon cancer, lung cancer, pancreatic cancer, lymphoma, and/or head and neck cancer.

In one embodiment, the disorder is breast cancer.

In one embodiment, the disorder is ovarian cancer.

In one embodiment, the disorder is prostate cancer.

In one embodiment, the disorder is colon cancer.

In one embodiment, the disorder is lung cancer.

In one embodiment, the disorder is pancreatic cancer.

In one embodiment, the disorder is lymphoma.

In one embodiment, the disorder is head and neck cancer.

In one embodiment, the cancer is associated with ALDH, especially ALDH1A1.

In one embodiment, the cancer is characterised by, or further characterised by, inappropriate activity of ALDH, especially ALDH1A1.

In one embodiment, the cancer is characterised by, or further characterised by, overexpression of ALDH, especially ALDH1A1.

In one embodiment, the cancer is characterised by, or further characterised by, an amplification of the ALDH1A1 gene.

With respect to treating, the anti-cancer effect may arise through one or more mechanisms, including but not limited to, radiotherapeutic effect (e.g., damaging cellular DNA by emission of ionizing radiation), inhibition of mechanisms for treatment resistance (i.e., enhancing the effect of a co-administered anti-cancer agent), the regulation of cell proliferation, the inhibition of cell cycle progression, the inhibition of angiogenesis (the formation of new blood vessels), the inhibition of metastasis (the spread of a tumour from its origin), the inhibition of cell migration (the spread of cancer cells to other parts of the body), the inhibition of invasion (the spread of tumour cells into neighbouring normal structures), the promotion of apoptosis (programmed cell death), death by necrosis, or induction of death by autophagy. The compounds described herein may be used in the treatment of the cancers described herein, independent of the mechanisms discussed herein.

As will be understood by the skilled person specific PQM-H compounds may be applied to different disorders when used for imaging as opposed to use in treatment.

### Treatment

The term “treatment,” as used herein in the context of treating a disorder, pertains generally to treatment of a human or an animal (e.g., in veterinary applications), in which some desired therapeutic effect is achieved, for example, the inhibition of the progress of the disorder, and includes a reduction in the rate of progress, a halt in the rate of progress, alleviation of symptoms of the disorder, amelioration of the disorder, and cure of the disorder. Treatment as a prophylactic measure (i.e., prophylaxis) is also included. For example, use with patients who have not yet developed the disorder, but who are at risk of developing the disorder, is encompassed by the term “treatment.”

For example, treatment includes the prophylaxis of cancer, reducing the incidence of cancer, alleviating the symptoms of cancer, etc.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

### Combination Therapies

The term “treatment” includes combination treatments and therapies, in which two or more treatments or therapies are combined, for example, sequentially or simultaneously. For example, the compounds described herein may also be used in combination therapies, e.g., in conjunction with other agents. Examples of treatments and therapies include chemotherapy (the administration of active agents, including, e.g., drugs, antibodies (e.g., as in immunotherapy), prodrugs (including, e.g., as in photodynamic therapy, GDEPT, ADEPT, etc.)); surgery; radiation therapy; photodynamic therapy; gene therapy; and controlled diets.

One aspect of the present invention pertains to a compound as described herein, in combination with one or more (e.g., 1, 2, 3, 4, etc.) additional therapeutic agents, as described below. The particular combination would be at the discretion of the physician who would select dosages using his common general knowledge and dosing regimens known to a skilled practitioner.

The agents (i.e., the compound described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., the compound described here, plus one or more other agents) may be formulated together in a single dosage form, or alternatively, the individual agents may be formulated separately and presented together in the form of a kit, optionally with instructions for their use.

Examples of additional agents/therapies that may be co-administered/combined with treatment with the PQM-H compounds described herein include the following:
an aromatase inhibitor, for example, exemestane (also known as Aromasin), letrozole (also known as Femara), anastrozole (also known as Arimidex), etc.;
an anti-estrogen, for example, faslodex (also known as Fulvestrant and ICI182780), tamoxifen (also known as Nolvadex), hydroxytamoxifen, etc.;
an anti-androgen, for example, an anti-androgen used in the treatment of prostate cancer, for example, flutamide, enzalutamide, apalutamide, bicalutamide, nilutamide, etc.;
a Her2 blocker, for example, herceptin, pertuzumab, lapatinib, etc.;
a cytotoxic chemotherapeutic agent, for example, a taxane (e.g., paclitaxel also known as Taxol; docetaxel also known as Taxotere), cyclophosphamide, an antimetabolite (e.g., carboplatin, capecitabine, gemcitabine, doxorubicin, epirubicin, 5-fluorouracil, etc.),
an agent stimulating the immune system, for example, a Toll-like receptor (TLR1-13) agonist, a Stimulator of Interferon Genes (STING) agonist, etc.;
a checkpoint inhibitor, for example, an inhibitor of PD1, PD1L, CTLA4, etc., for example, pembrolizumab, atezolizumab, ipilimumab, etc.;
a DNA repair inhibitor, for example, a PARP inhibitor, for example, Olaparib, Niraparib, etc.

Thus, in one embodiment, the treatment further comprises treatment (e.g., simultaneous or sequential treatment) with a further active agent which is, e.g., an aromatase inhibitor, an anti-estrogen, an anti-androgen, a Her2 blocker, a cytotoxic chemotherapeutic agent, an agent stimulating the immune system, a checkpoint inhibitor, a DNA repair inhibitor, etc.

In one embodiment, two or more different PQM-H compounds may be administered simultaneously or sequentially.

In one embodiment, a first PQM-H compound comprising a radioisotope that decays by positron emission, or gamma decay (e.g., ¹⁸F, ¹²⁵I, etc.) is administered to a subject; then a second PQM-H compound comprising a radioisotope that decays by alpha particle emission, or beta particle emission (e.g., ¹³¹I, ²¹¹At, etc.) may be administered to the subject. In this way, the status of the disorder (including ALDH presence, e.g., ALDH1A1 presence) can be determined, e.g., via a method of medical imaging described herein after administration of the first PQM-H compound; then the disorder can be treated by radiotherapy via the administration of the second PQM-H compound.

Optionally, after administration of the second PQM-H compound an additional dose of the first PQM-H compound is administered to the subject. In this way, the effect of the therapy can be actively monitored via the second administration of the first PQM-H compound, for example by a method of medical imaging as described herein.

The subject may be administered additional cycles of treatment with the second PQM-H compound, and monitoring of therapy effect with the first PQM-H compound. In these cycles, the first and second PQM-H compounds may be administered simultaneously or sequentially.

In one embodiment, the second PQM-H compound comprising a radioisotope that decays by alpha particle emission, or beta particle emission (e.g., ¹³1, ²¹¹At, etc.) may be administered in combination with (simultaneously or sequentially) the first PQM-H compound comprising a radioisotope that decays by positron emission, or gamma decay (e.g., ¹⁸F, ¹²⁵I, etc.). In this way, the second PQM-H compound can treat a disorder by radiotherapy and the effect of the therapy can be actively monitored via the first PQM-H compound, for example by a method of medical imaging as described herein.

### Processes For Preparing PQM-H Compounds From PQM-P Compounds

Another aspect of the invention pertains to a process for preparing a PQM-H compound from a PQM-P compound.

The PQM-P compounds described herein are useful in the preparation of PQM-H compounds. For example, the PQM-P compounds may undergo a reaction in which the PQM-P compound is contacted with a source of radioisotope under conditions to replace the leaving group on the PQM-P compound with a radioisotope from the source of radioisotope, such that the reaction product is a PQM-H compound.

In one embodiment, the reaction is a nucleophilic radiohalogenation.

In one embodiment, the reaction is a nucleophilic radiofluorination.

In one embodiment, the reaction is a nucleophilic radioiodination.

In one embodiment the source of radioisotope is a source of ¹⁸F, ¹²³I, ¹²⁴I, ¹²⁵I, ¹³¹I, or ²¹¹At.

In one embodiment the source of radioisotope is a source of ¹⁸F, ¹²³I, ¹²⁴I, ¹²⁵I, or ¹³¹I.

In one embodiment the source of radioisotope is a source of ¹⁸F or ¹²⁵I.

In one embodiment the source of radioisotope is a source of ¹²⁵I.

In one embodiment the source of radioisotope is a source of ¹⁸F.

In one embodiment, the reaction takes place in the presence of a catalyst.

In one embodiment, the reaction takes place in the presence of a copper catalyst.

In one embodiment, the catalyst is Tetrakis(pyridine)copper(II) bis(trifluoromethanesulfonate).

In one embodiment, the catalyst is Cu(OCOCF₃)₂.

In one embodiment, the reaction is heated at a temperature between about 20° C. and about 150° C.

In one embodiment, the reaction is heated at a temperature between about 40° C. and about 140° C.

In one embodiment, the reaction is heated at a temperature between about 50° C. and about 130° C.

In one embodiment, the reaction is heated at a temperature between about 60° C. and about 120° C.

In one embodiment, the reaction is heated at a temperature between about 80° C. and about 110° C.

In one embodiment, the reaction is heated at a temperature of about 80° C.

In one embodiment, the reaction is heated at a temperature of about 110° C.

In one embodiment, the reaction is heated at room temperature (e.g., about 25° C.).

In one embodiment, the reaction is conducted in less than 6 h.

In one embodiment, the reaction is conducted in less than 5 h.

In one embodiment, the reaction is conducted in less than 4 h.

In one embodiment, the reaction is conducted in less than 3 h.

In one embodiment, the reaction is conducted in less than 2 h.

In one embodiment, the reaction is conducted in less than 1 h.

In one embodiment, the reaction is conducted in less than 45 min.

In one embodiment, the reaction is conducted in less than 30 min.

In one embodiment, the reaction is conducted in about 20 min.

In one embodiment, the reaction is stirred for about 1 min to about 3 h.

In one embodiment, the reaction is stirred for about 5 min to about 1 h.

In one embodiment, the reaction is stirred for about 10 min to about 30 min.

In one embodiment, the reaction is stirred for about 20 min.

### Other Uses

The PQM-H compounds described herein may also be used as cell culture additives to inhibit ALDH (e.g., ALDH1A1).

The PQM-H compounds described herein may also be used as part of an in vitro assay, for example, in order to determine whether a candidate host is likely to benefit from treatment with the compound in question.

The PQM-H compounds described herein may also be used as a standard, for example, in an assay, in order to identify other active compounds, other ALDH1A1 inhibitors, etc. For example, the PQM-H compounds described herein may be useful as a standard in a competition binding assay.

### Kits

One aspect of the invention pertains to a kit comprising (a) a PQM-H compound as described herein, or a composition comprising an PQM-H compound as described herein, e.g., preferably provided in a suitable container and/or with suitable packaging; and (b) instructions for use, e.g., written instructions on how to administer the compound or composition.

The written instructions may also include a list of indications for which the active ingredient is a suitable treatment.

Another aspect of the invention pertains to a kit comprising (a) a PQM-P compound as described herein, or a composition comprising an PQM-P compound as described herein, e.g., preferably provided in a suitable container and/or with suitable packaging; and (b) instructions for use, e.g., written instructions on how to generate a PQM-H compound from the PQM-P compound.

### Routes of Administration

The PQM-H compound or pharmaceutical composition comprising PQM-H compound may be administered to a subject by any convenient route of administration, whether systemically/peripherally or topically (i.e., at the site of desired action).

Examples of routes of administration include oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

### Formulations

While it is possible for a PQM-H compound to be administered alone, it is preferable to present it as a pharmaceutical formulation (e.g., composition, preparation, medicament) comprising at least one PQM-H compound, as described herein, together with one or more other pharmaceutically acceptable ingredients well known to those skilled in the art, including pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising mixing at least one PQM-H compound, as described herein, together with one or more other pharmaceutically acceptable ingredients well known to those skilled in the art, e.g., carriers, diluents, excipients, etc. If formulated as discrete units (e.g., vials, ampoules, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005.

The formulations may be prepared by any methods well known in the art of pharmacy. Such methods include the step of bringing into association the compound with a carrier, which constitutes one or more accessory ingredients. In general, the formulations are prepared by uniformly and intimately bringing into association the compound with carriers (e.g., liquid carriers, finely divided solid carrier, etc.), and then shaping the product, if necessary.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or mixed with one or more other pharmaceutically acceptable ingredients. The compound may be presented in a liposome or other microparticulate which is designed to target the compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the compound in a flavored basis, usually sucrose and acacia or tragacanth. Pastilles typically comprise the compound in an inert matrix, such as gelatin and glycerin, or sucrose and acacia. Mouthwashes typically comprise the compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Tablets may be made by moulding in a suitable machine a mixture of the powdered compound moistened with an inert liquid diluent. The tablets may optionally be coated or scored and may be formulated so as to provide slow or controlled release of the compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the compound and a paraffinic or a water-miscible ointment base.

Creams are typically prepared from the compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the compound through the skin or other affected areas. Examples of such dermal penetration enhancers include dimethylsulfoxide and related analogues.

Emulsions are typically prepared from the compound and an oily phase, which may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for ocular administration include eye drops wherein the compound is dissolved or suspended in a suitable carrier, especially an aqueous solvent for the compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the compound, such carriers as are known in the art to be appropriate.

Formulations suitable for parenteral administration (e.g., by injection) include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additionally contain other pharmaceutically acceptable ingredients, such as anti-oxidants, buffers, preservatives, stabilisers, bacteriostats, suspending agents, thickening agents, and solutes, which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/mL to about 10 μg/mL, for example from about 10 ng/mL to about 1 μg/mL. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the PQM-H compounds, and compositions comprising the PQM-H compounds, can vary from patient to patient. Determining the optimal dosage will generally involve the balancing of the level of therapeutic benefit against any risk or deleterious side effects. Dosage for therapeutic effects may differ from the dosage required for diagnostic effects. The selected dosage level will depend on a variety of factors including the activity of the particular PQM-H compound, the route of administration, the time of administration, the rate of excretion of the PQM-H compound, the duration of the treatment, other drugs, compounds, and/or materials used in combination, the severity of the disorder, and the species, sex, age, weight, condition, general health, and prior medical history of the patient. The amount of PQM-H compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In the case of the use of PQM-H compounds for imaging, the dose is in an amount sufficient to provide a signal for medical imaging.

In the case of the use of a PQM-H compound for therapy, the dose is an amount sufficient to provide sufficient concentration of the PQM-H compound to provide a therapeutic benefit. For example, for targeted radiotherapy, the dose of the PQM-H compound is an amount to provide a sufficient level of ionizing radiation at the desired site.

As radioactive compounds, dosage of PQM-H compounds is typically measured as a dosage of radioactivity, for example, as measured in Curie (Ci) or in Becquerel (Bq). Radioactive dosage also varies based on the radioisotope which is present in the PQM-H compound.

### EXAMPLES

### Chemical Synthesis

### Abbreviations

aq: aqueous;
br: broad;
ca.: circa;
Cu(OCOCF₃)₂: Copper(II) trifluoroacetate hydrate;
d: doublet;
DCM: dichloromethane;
DEEM: diethyl ethoxymethylenemalonate;
dioxane: 1,4-dioxane;
DIPEA: diisopropylethylamine;
DMA: N,N-Dimethylacetamide;
DMF: N,N-dimethylformamide;
Eq: equivalents;
Et₃N: triethylamine;
EtOAc: ethyl acetate;
EtOH: ethanol;
h: hours;
HATU: N-[(Dimethylamino)-1H-1,2,3-triazolo-[4,5-b]pyridin-1-ylmethylene]-N-methylmethanaminium hexafluorophosphate N-oxide;
HCl: hydrochloric acid;
HPLC: high performance liquid chromatography;
K₂CO₃: potassium carbonate;
KOAc: potassium acetate;
KOH: potassium hydroxide;
Kryptofix 222: 4,7,13,16,21,24-Hexaoxa-1,10-diazabicyclo[8.8.8]hexacosane;
LCMS: liquid chromatography-mass spectrometry;
LiCl: lithium chloride;
m: multiplet;
M: molar, molecular ion;
MBq: megabecquerel;
MeCN: actetonitrile;
MeOH: methanol;
MgSO₄: magnesium sulfate;
min: minutes;
mmol: millimoles;
MS: mass spectrometry;
NaHCO₃: sodium bicarbonate;
NaI: sodium iodide;
NaOH: sodium hydroxide;
Na₂SO₄: sodium sulfate;
NMR: nuclear magnetic resonance;
PBS: phosphate buffered saline;
Pd₂dba₃: Tris(dibenzylideneacetone)dipalladium(0);
PdCl₂(dppf)-DCM: [1,1′-Bis(diphenylphosphino)ferrocene]dichloropalladium(II), complex with dichloromethane;
PdCl₂(dtbpf): [1,1′-Bis(diphenylphosphino)ferrocene]dichloropalladium(II);
PE: petroleum ether;
q: quartet;
RCY: radiochemical yield;
rotovap: rotary evaporator;
RT: room temperature (ca. 20° C.);
R_{T}: retention time;
brs: broad singlet;
s: singlet, solid;
sat.: saturated;
t: triplet;
THF: tetrahydrofuran;
TLC: thin layer chromatography;
XPhos: 2-Dicyclohexylphosphino-2′,4′,6′-triisopropylbiphenyl.

Other abbreviations are intended to convey their generally accepted meaning.

Nomenclature of structures was generated using ‘Structure to Name’ conversion from ChemDraw® Professional 17 (PerkinElmer).

### General Synthetic Methods

Methods for the chemical synthesis of the PQM-P and PQM-H compounds are described herein. These and/or other well-known methods may be modified and/or adapted in known ways in order to provide alternative or improved methods of synthesis of the PQM compounds.

In a first method, the synthesis starts with the quinoline derivative I-1. Suzuki coupling (a) affords the corresponding 4-substituted-quinoline 1-2. Hydrolysis of the aryl ester affords the intermediate 1-3. The latter is used in an amide coupling reaction with an appropriately substituted piperazine to yield the chloroquinoline intermediate 1-4. Miyaura borylation affords the boron pinacol ester compound 1-5 (a PQM-P compound). Final copper-catalysed nucleophilic radiohalogenation of aryl boronic ester affords compound 1-6 (a PQM-H compound).

This method is illustrated in the following chemical scheme.

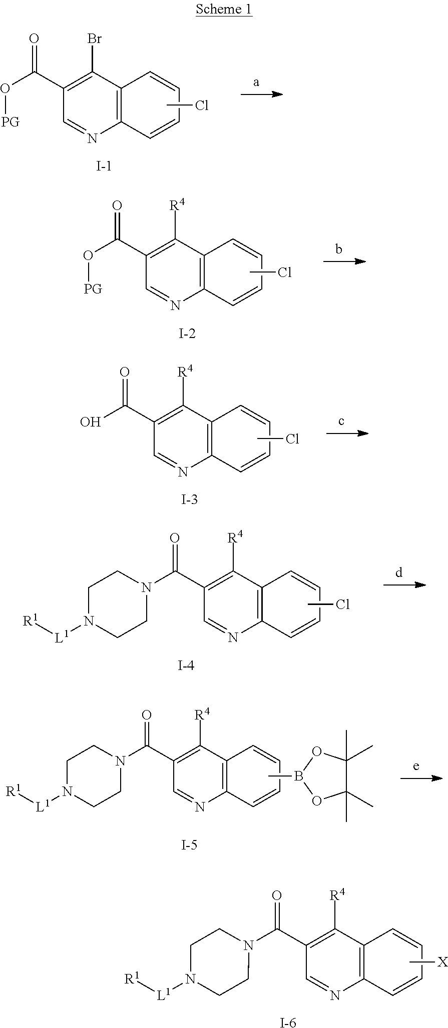

Scheme 1: general methods of synthesis of PQM-P and PQM-H compounds. Representative reactions conditions for the above scheme are as follows: (a) R⁴—BPin, PdCl₂(dtbpf), K₂CO₃, DMF, 90° C.; (b) NaOH, THF/MeOH, 50° C.; (c) R^{L}-L¹-piperazine, DIPEA, HATU, DMF, RT; (d) Bis(pinacolato)diborane, KOAc, Pd(cat) THF/DMF, 110° C.; (e) Cu(cat), Na/KX (X=radiohalide), solvent.

### Chemical Synthesis Examples

The following examples are provided solely to illustrate the present invention and are not intended to limit the scope of the invention, as described herein.

### General Experimental Conditions

All starting materials and solvents were obtained either from commercial sources or prepared according to the literature citation. [¹⁸F]Fluoride was produced by a GE PETrace cyclotron by 16 MeV irradiation of enriched [¹⁸O]H₂O target, supplied by Alliance Medical Radiopharmacy Ltd (London, UK) or St Thomas' Hospital (London, UK) in approximately 3 mL of water. [¹⁸F]Fluoride was used without further purification. Reductant free [¹²⁵I]NaI was purchased from PerkinElmer (product number NEZ033A) as a carrier-free, 10-5M NaOH solution (pH 8-11). Radioactivity was measured in a CRC-25R dose calibrator (Capintec, Inc). Reaction mixtures were magnetically stirred unless otherwise indicated.

Column chromatography was performed on an automated flash chromatography system, Biotage Isolera One, using Biotage® Sfar or Biotage® Sfar Bio C18 Duo pre-packed silica (60 μm, 20 μm) cartridges, unless otherwise indicated.

¹H NMR and ¹³C NMR spectra were recorded using a Bruker Avance III spectrometer 400 MHz. Chemical shifts are expressed in parts per million using either the central peaks of the residual protic solvent or an internal standard of tetramethylsilane as references. The spectra were recorded at ambient temperature unless otherwise stated.

Analytical and semi-preparative RP-HPLC were performed with an Agilent 1200 HPLC system equipped with a 1200 Series Diode Array Detector and a Raytest GABI Star NaI(TI) scintillation detector (energy window 400-700 keV). Isolated radiochemical yield (RCY) refers to the activity of the pure tracer isolated after HPLC divided by the initial activity of [¹⁸F]fluoride in [¹⁸O]H2O or [¹²⁵I]NaI in NaOH solution used for the labelling. RCYs are given decay corrected. Radiochemical purity refers to the proportion of the total radioactivity in the sample, which is present as the desired radiotracer, as measured by radio-HPLC.

### Analytical Methods

Method 1—LCMS 15 min method:
Column: ZORBAX Eclipse, XDB-C18, 80 Å, 1.8 μm, 4.6×50 mm
Detection: UV at 254 nm
MS ionisation: Electrospray
Solvent A: Water
Solvent B: MeCN
Flow Rate: 1.0 ml/min

Method 1 - Gradient

| Time | % A | % B |
|---|---|---|
| 0.0 | 95.0 | 5.0 |
| 2.0 | 95.0 | 5.0 |
| 11.00 | 5.0 | 95.0 |
| 12.00 | 5.0 | 95.0 |
| 12.10 | 95.0 | 5.0 |
| 15.00 | 95.0 | 5.0 |

Method 2—LCMS 15 min method:
Column: ZORBAX Eclipse, XDB-C18, 80 Å, 1.8 μm, 4.6×50 mm
Detection: UV at 254 nm
MS ionisation: Electrospray
Solvent A: Water
Solvent B: MeCN
Flow Rate: 1.0 ml/min

Method 2 - Gradient

| Time | % A | % B |
|---|---|---|
| 0.0 | 20.0 | 80.0 |
| 2.0 | 20.0 | 80.0 |
| 6.00 | 1.0 | 99.0 |
| 13.00 | 1.0 | 99.0 |
| 14.10 | 95.0 | 5.0 |
| 15.00 | 95.0 | 5.0 |

Method 3—LCMS 25 min method for Radiochemicals:
Column: Zorbax® HPLC Column C18 (octadecyl), 5 μm, 25 cm×4.6 mm or
Detection: UV at 254 nm
Solvent A: Water
Solvent B: MeOH
Flow Rate: 3.0 ml/min

Method 3 - Gradient

| Time | % A | % B |
|---|---|---|
| 0.0 | 40.0 | 60.0 |
| 12.0 | 2.0 | 98.0 |
| 18.0 | 2.0 | 98.0 |
| 20.00 | 50.0 | 50.0 |

### Synthesis 1—Synthesis of Precursor Compounds (PQM-P Compounds)

PQM-P-001, PQM-H-001 and PQM-H-002 were synthesized via the synthetic route below (Scheme 2):

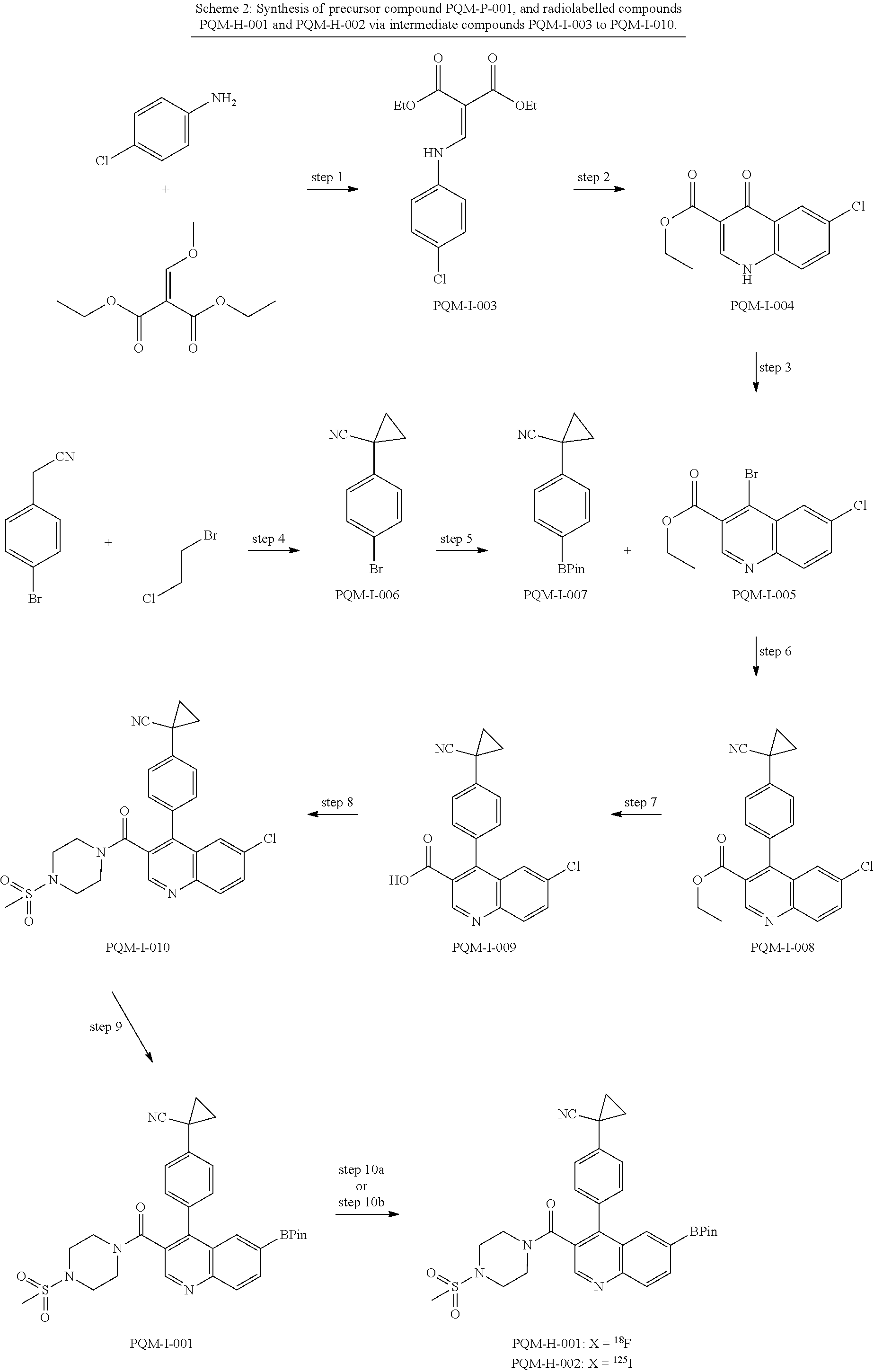

### Synthesis 1a—Synthesis of 1-[4-[3-(4-methylsulfonylpiperazine-1-carbonyl)-6-(4,4,5,5-tetramethyl-1,3,2-dioxaborolan-2-yl)-4-quinolyl]phenyl]cyclopropanecarbonitrile

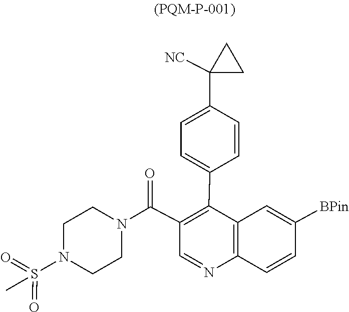

### Step 1—Diethyl 2-(((4-chlorophenyl)amino)methylene)malonate

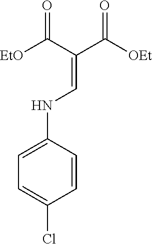 [SMILES: CCOC(=O)C(=CNc1ccc(Cl)cc1)C(=O)OCC]

A 100 mL round-bottomed flask was charged with 4-chloroaniline (4.13 g, 1 Eq., 32.4 mmol) and diethyl 2-(ethoxymethylene)malonate (7.00 g, 1 Eq., 32.4 mmol). Then the mixture was stirred at 130° C. for 2 h. After consumption of starting compound (TLC monitoring) reaction was cooled to room temperature and crude purified by silica gel column chromatography eluting with 0-10% EtOAc/PE. To yield diethyl 2-(((4-chlorophenyl)amino)methylene)malonate as a white solid (94% yield).

¹H NMR (400 MHz, CDCl3): δ[ppm]=1.33 (t, J=7.1 Hz, 3H, CH3CH2), 1.38 (t, J=7.1 Hz, 3H, CH3CH2), 4.25 (q, J=7.1 Hz, 2H, CH3CH2), 4.30 (q, J=7.1 Hz, 2H, CH3CH2), 7.07 (d, J=8.8 Hz, 2H, Ar—H), 7.34 (d, J=8.8 Hz, 2H, Ar—H), 8.45 (d, J=13.6 Hz, 1H, CH), 11.00 (d, J=13.5 Hz, 1H, NH).

¹³C NMR (100 MHz, CDCl3): δ[ppm]=14.4, 14.5, 60.3, 60.6, 94.4, 118.4, 130.1, 130.2, 138.1, 151.7, 165.7, 169.1

### Step 2—Ethyl 6-chloro-4-oxo-1,4-dihydroquinoline-3-carboxylate

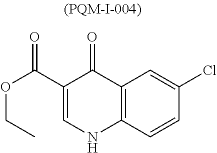 [SMILES: CCOC(=O)c1c[nH]c2ccc(Cl)cc2c1=O]

A 250 mL round-bottomed flask was charged with diethyl 2-(((4-chlorophenyl)amino)methylene)malonate (4.00 g, 1 Eq., 13.4 mmol) and diphenylether (15 mL). Then the mixture was stirred at 260° C. for 2 hours. After consumption of starting compound (TLC monitoring) reaction was cooled to room temperature and diluted with 100 mL hexane. Formed precipitate was collected by filtration through a glass filter and dried under high vacuo to yield ethyl 6-chloro-4-oxo-1,4-dihydroquinoline-3-carboxylate as white solid (70%) and used in the next step without further purification.

### Step 3—Ethyl 4-bromo-6-chloroquinoline-3-carboxylate

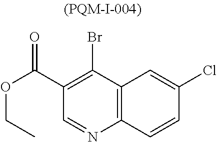 [SMILES: CCOC(=O)c1cnc2ccc(Cl)cc2c1Br]

A 100 mL round-bottomed flask was charged with ethyl 6-chloro-4-oxo-1,4-dihydroquinoline-3-carboxylate (2.1 g, 1 Eq., 8.3 mmol) and phosphoryl tribromide (12 g, 5 Eq., 42 mmol). Then the mixture was stirred at 125° C. for 3 hours. After consumption of starting compound (TLC monitoring) reaction was cooled to 0° C. and quenched with ice slush and NaHCO₃, then extracted with EtOAc (3×20 mL). The combined organic layers were washed with brine (3×20 ml), dried over Na₂SO₄, filtered and the solvent was eliminated under vacuo to yield the title compound obtained as a white solid (84% yield).

¹H NMR (400 MHz, CDCl3): δ[ppm]=1.47 (t, J=7.2 Hz, 3H, CH3CH2), 4.51 (q, J=7.2 Hz, 2H, CH3CH2), 7.75 (dd J=9.0, 2.3 Hz, 1H, Ar—H), 8.06 (d, J=9.0 Hz, 1H, Ar—H), 8.39 (d, J=2.3 Hz, 1H, Ar—H), 9.05 (s, 1H, Ar—H).

¹³C NMR (100 MHz, CDCl3, 298K): δ[ppm]=14.4, 62.6, 127.3, 127.4, 128.6, 131.7, 132.8, 133.6, 135.1, 147.7, 149.9, 165.0.

### Step 4—1-(4-bromophenyl)cyclopropane-1-carbonitrile

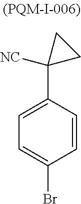 [SMILES: N#CC1(c2ccc(Br)cc2)CC1]

A 100 mL round-bottomed flask was charged with 2-(4-bromophenyl)acetonitrile (5.00 g, 1 Eq., 25.5 mmol), 1-bromo-2-chloroethane (7.31 g, 2 Eq., 51.0 mmol), tetrabutylammonium bromide (1.64 g, 0.2 Eq., 5.10 mmol), potassium hydroxide (7.16 g, 5 Eq., 128 mmol) and toluene/sat. KOH (aq.) (20:12). Then the mixture was stirred at 50° C. overnight. After consumption of starting compound (TLC monitoring) the reaction was cooled to room temperature and quenched with water, then extracted with DCM (3×20 mL). The combined organic layers were washed with brine (3×20 ml), dried over Na₂SO₄, filtered and the solvent was eliminated under vacuo to give a residue that was purified by silica gel column chromatography eluting with 5-20% EtOAc/n-heptane to yield the title compound obtained as a white solid (67% yield).

¹H NMR (400 MHz, CDCl3): δ[ppm]=1.38 (m, 2H, CH2), 1.73 (m, 2H, CH2), 7.16 (d, J=8.6 Hz, 2H, Ar—H), 7.47 (d, J=8.6 Hz, 2H, Ar—H).

¹³C NMR (100 MHz, CDCl3): δ[ppm]=13.6, 18.4, 121.7, 122.2, 127.6, 132.1, 135.3

### Step 5—1-(4-(4,4,5,5-tetramethyl-1,3,2-dioxaborolan-2-yl)phenyl)cyclopropane-1-carbonitrile

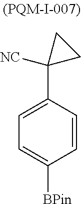

A 100 mL round bottom flask was charged with 1-(4-bromophenyl)cyclopropane-1-carbonitrile (4.30 g, 1 Eq. 19.36 mmol), 4,4,4′,4′,5,5,5′,5′-octamethyl-2,2′-bi(1,3,2-dioxaborolane) (14.75 g, 3.0 Eq., 58.08 mmol), XPhos (461.5 mg, 0.05 Eq.), Pd₂dba₃ (443.2 mg, 0.025 Eq.), KOAc (5.7 g, 3 Eq.) and 1,4-dioxane (60 mL). The reaction mixture was stirred at 110° C. for an hour. After consumption of starting compound (TLC monitoring) the reaction mixture was cooled down to room temperature, diluted with ethyl acetate (25 mL) and then filtered through celite. The solvents were removed under reduced pressure and the obtained oily residue was purified via flash chromatography eluting with 5-10% EtOAc n-hexane. The title compound was obtained as white solid (4.3 g, 91%).

¹H NMR (400 MHz, CDCl3) δ 1.28 (s, 12H, CH3), 1.37 (m, 2H, CH2), 1.69 (m, 2H, CH2), 7.23 (d, J=2H, Ar—H), 7.72 (d, J=7.8 Hz, 2H, Ar—H).

¹³C NMR (100 MHz, CDCl3) δ 14.1, 18.9, 25.0, 83.6, 84.1, 122.5, 124.8, 135.5, 139.2.

### Step 6—Ethyl 6-chloro-4-(4-(1-cyanocyclopropyl)phenyl)quinoline-3-carboxylate

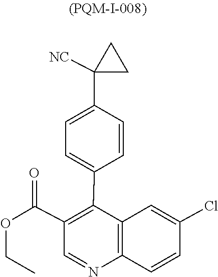 [SMILES: CCOC(=O)c1cnc2ccc(Cl)cc2c1-c1ccc(C2(C#N)CC2)cc1]

A 50 mL round-bottom flask was charged with ethyl 4-bromo-6-chloroquinoline-3-carboxylate (1.5 g, 1 Eq., 4.8 mmol), 1-(4-(4,4,5,5-tetramethyl-1,3,2-dioxaborolan-2-yl)phenyl)cyclopropane-1-carbonitrile (1.7 g, 1.3 Eq., 6.2 mmol), PdCl₂(dtbpf) (310 mg, 0.1 Eq.), potassium carbonate (2.0 g, 3 Eq., 14 mmol) and 20 mL of dry DMF. Then the reaction mixture was stirred at 90° C. for 3 h. After consumption of starting compound (TLC monitoring) the reaction mixture was cooled down to room temperature and the DMF was removed under reduced pressure. The residue was diluted with ethyl acetate (50 mL), rinsed with LiCl (sat. aq.) (3×15 mL), water (3×20 mL) the organic layer was washed with brine (3×10 ml), dried over Na₂SO₄, filtered and the solvent was eliminated under vacuo to give a residue that was purified by silica gel column chromatography eluting 20% EtOAc/n-hexane to afford the title compound as a white solid (56% yield).

¹H NMR (400 MHz, CDCl3): δ[ppm]=1.05 (t, J=7.1 Hz, 3H, CH3CH2), 1.55 (m, 2H, CH2), 1.84 (m, 2H, CH2), 4.15 (q, J=7.2 Hz, 2H, CH3CH2), 7.28 (d, J=8.2 Hz, 2H, Ar—H), 7.45 (d, J=8.2 Hz, 2H, Ar—H), 7.49 (d, J=2.3 Hz, 1H, Ar—H), 7.73 (dd J=9.0, 2.3 Hz, 1H, Ar—H) 8.14 (d, J=9.0 Hz, 1H, Ar—H), 9.33 (s, 1H, Ar—H).

¹³C NMR (100 MHz, CDCl3): δ[ppm]=13.86, 13.99, 18.83, 61.7, 122.4, 124.1, 125.7, 126.0, 127.9, 129.5, 131.3, 132.3, 133.8, 135.3, 136.7, 147.5, 148.3, 150.3, 166.0.

### Step 7—6-chloro-4-(4-(1-cyanocyclopropyl)phenyl)quinoline-3-carboxylic acid

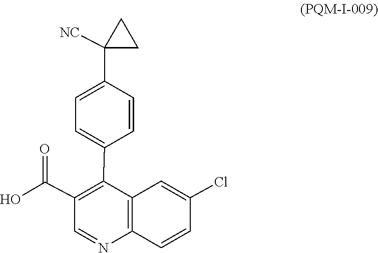 [SMILES: N#CC1(c2ccc(-c3c(C(=O)O)cnc4ccc(Cl)cc34)cc2)CC1]

A 25 mL flask was charged with ethyl 6-chloro-4-(4-(1-cyanocyclopropyl)phenyl)quinoline-3-carboxylate (0.9 g, 1 Eq., 2 mmol) and THF/MeOH (2:8, 10 mL). Then, sodium hydroxide (0.3 g, 3 Eq, 7 mmol) in 1 mL water was added dropwise to the reaction mixture and stirred at 50° C. for 3 h. After consumption of starting compound (TLC monitoring) the reaction mixture was cooled down to 0° C. and the pH was adjusted to 5 via adding cold 1M HCl (aq.) solution to form a heavy precipitate. The precipitate was filtered, washed with water (3×10 mL) and dried under vacuo to afford the title compound as a white solid (69% yield).

¹H NMR (400 MHz, DMSO-d6): δ[ppm]=1.65 (m, 2H, CH2), 1.85 (m, 2H, CH2), 7.38 (m, 3H, Ar—H), 7.48 (d, J=8.4 Hz, 2H, Ar—H), 7.90 (d, J=8.9 Hz, 1H, Ar—H), 8.17 (d, J=8.9, 1H, Ar—H) 8.14 (d, J=9.0 Hz, 1H, Ar—H), 9.2 (s, 1H, Ar—H), 13.3 (brs, 1H, OH).

### Step 8—1-(4-(6-chloro-3-(4-(methylsulfonyl)piperazine-1-carbonyl)quinolin-4-yl)phenyl)cyclopropane-1-carbonitrile

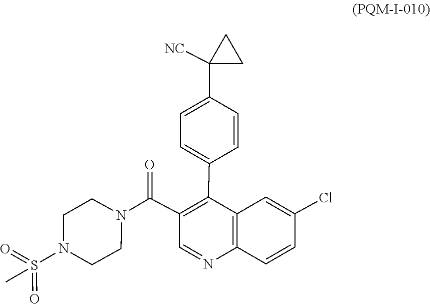 [SMILES: CS(=O)(=O)N1CCN(C(=O)c2cnc3ccc(Cl)cc3c2-c2ccc(C3(C#N)CC3)cc2)CC1]

A 10 mL round-bottom flask was charged with 6-chloro-4-(4-(1-cyanocyclopropyl)phenyl)quinoline-3-carboxylic acid (0.80 g, 1 Eq., 2.3 mmol), HATU (0.96 g, 1.1 Eq), DIPEA (1.5 g, 5 Eq., 11 mmol) and DMF 10 mL, and stirred for 5 minutes at RT. Then 1-(methylsulfonyl)piperazine (0.56 g, 1.5 Eq., 3.4 mmol) was added and the reaction mixture was stirred for 4 hours at RT. After consumption of starting compound (TLC monitoring) the reaction was quenched at 0° C. with 1 M HCl (aq.) solution and extracted with EtOAc (3×10 mL). The organic layers were combined and washed with brine (3×10 mL), dried over Na₂SO₃, filtered and the solvent was eliminated under vacuo to give a residue that was purified by silica gel column chromatography eluting with 2-8% MeOH/DCM afford the as a white solid (40% yield)

¹H NMR (400 MHz, CDCl₃): δ[ppm]=1.55 (m, 2H, CH2), 1.85 (m, 2H, CH2), 2.37 (m, 1H), 2.70 (s, 3H, CH3), 2.82 (m, 1H), 2.97 (m, 2H), 3.21 (m, 2H), 3.69 (m, 2H), 7.39 (d, J=8.1 Hz, 1H, Ar—H), 7.45 (d, J=8.1 Hz, 1H, Ar—H) 7.54 (brs, 2H, Ar—H), 7.68 (d, J=2.2 Hz, 1H, Ar—H), 7.72 (dd, J=9.0, 2.3 Hz, 1H, Ar—H), 8.13 (d, J=9.0 Hz, 1H, Ar—H), 8.85 (s, 1H, Ar—H).

¹³C NMR (100 MHz, CDCl₃): δ[ppm]=13.9, 18.8, 35.1, 41.2, 45.4, 45.6, 46.4, 122.1, 124.9, 126.1, 126.6, 126.7, 128.6, 130.1, 131.2, 131.6, 131.7, 133.6, 134.2, 138.1, 142.4, 147.1, 147.9, 167.1

### Step 9—1-[4-[3-(4-methylsulfonylpiperazine-1-carbonyl)-6-(4,4,5,5-tetramethyl-1,3,2-dioxaborolan-2-yl)-4-quinolyl]phenyl]cyclopropanecarbonitrile

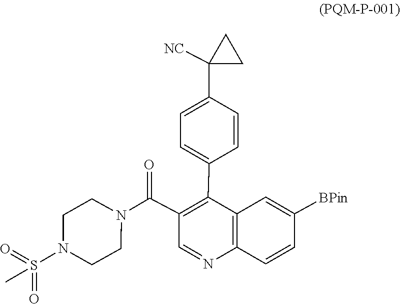

A 8 mL Borax tube was charged with 1-(4-(6-chloro-3-(4-(methylsulfonyl)piperazine-1-carbonyl)quinolin-4-yl)phenyl)cyclopropane-1-carbonitrile (0.150 g, 1 Eq, 303 μmol), Bis(pinacolato)diborane (154 mg, 2 Eq, 606 μmol), potassium acetate (89.2 mg, 56.8 μL, 3 Eq, 909 μmol), methanesulfonato(2-dicyclohexylphosphino-2′,6′-dimethoxy-1,1′-biphenyl)(2′-methylamino-1,1′-biphenyl-2-yl)palladium(II) dichloromethane adduct (12.1 mg, 0.05 Eq, 15.2 μmol) and 4 mL THF/DMF (2.5/1.5). The reaction mixture was heated in a closed system via a microwave synthesizer (CEM Discovery SP) at 110° C. for 20 min. Next, the vessed was cooled via air jet cooling. Then, all solvent was eliminated under vacuo to give a residue that was purified by C-18 reversed phase flash column chromatography eluting with 0-100% Water/MeCN to afford the title compound as a white solid (39% yield)

¹H NMR (400 MHz, CDCl₃): δ[ppm]=1.25 (s, 12H, CH3), 1.55 (m, 2H, CH2), 1.86 (m, 2H CH2), 2.36 (m, 1H), 2.71 (s, 3H, CH3), 2.84 (m, 1H), 2.97 (m, 2H), 3.22 (m, 2H), 3.69 (brs, 2H), 7.39 (d, J=8.1 Hz, 1H, Ar—H), 7.45 (d, J=8.1 Hz, 1H, Ar—H), 7.55 (brs, 2H, Ar—H), 7.69 (m, 1H, Ar—H), 7.73 (m, 1H, Ar—H), 8.14 (d, J=8.9 Hz, 1H, Ar—H), 8.85 (s, 1H, Ar—H).

¹³C NMR (100 MHz, CDCl₃): δ[ppm]=13.9, 18.8, 24.7, 25.2, 35.2, 41.3, 45.4, 45.7, 46.4, 83.6, 122.1, 124.9, 126.1, 126.6, 126.8, 128.6, 130.1, 131.2, 131.6, 133.6, 134.3, 138.1, 142.6, 147.0, 147.9, 167.0.

### Synthesis 1b—Synthesis of 1-[4-[3-(4-Methylsulfonylpiperazine-1-carbonyl)-8-nitro-6-(4,4,5,5-tetramethyl-1,3,2-dioxaborolan-2-yl)-4-quinolyl]phenyl]cyclopropanecarbonitrile

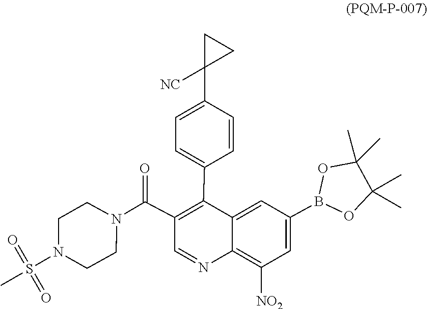

Following the procedure for PQM-P-001 using 4-chloro-2-nitroaniline (1 Eq) and DEEM (1.2 Eq) as starting reagents, formed residue was purified by C-18 reverse-phase flash column chromatography eluting with 0-100% Water/MeCN and the title compound was obtained as white solid.

¹H NMR (400 MHz, CDCl₃): δ 1.34 (s, 12H), 1.57 (m, 2H), 1.88 (t, 2H), 2.49 (m, 1H), 2.72 (s, 3H), 2.89 (m, 3H), 3.17 (brs, 1H), 3.52 (brs, 1H), 3.81 (brs, 1H), 7.40 (m, 2H), 7.55 (d, J=7.2, 2H), 8.39 (d, J=24.9 Hz, 2H), 8.98 (s, 1H).

¹³C NMR (100 MHz, CDCl₃): δ 13.8, 19.1, 24.8, 35.1, 41.1, 45.2, 45.4, 46.1, 77.2, 85.0, 121.8, 126.0, 126.1, 128.8, 129.5, 130.2, 131.1, 132.9, 136.8, 138.3, 141.2, 143.9, 148.3, 150.4, 166.3.

### Synthesis 1c—Synthesis of 1-[4-[3-(4-Methylsulfonylpiperazine-1-carbonyl)-7-(4,4,5,5-tetramethyl-1,3,2-dioxaborolan-2-yl)-4-quinolyl]phenyl]cyclopropanecarbonitrile

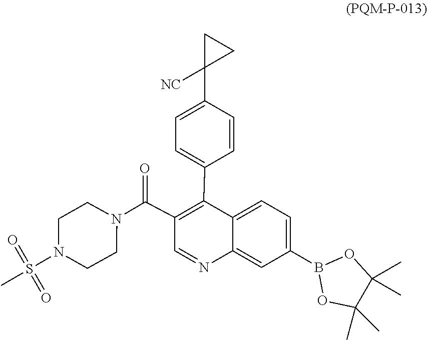

Following the procedure for PQM-P-001 using 3-chloroaniline (1 Eq) and DEEM (1.2 Eq) as starting reagents, formed residue was purified by C-18 reverse-phase flash column chromatography eluting with 0-100% Water/MeCN and the title compound was obtained as white solid.

¹H NMR (400 MHz, CDCl₃): δ 1.39 (s, 12H), 1.53 (m, 2H), 1.84 (t, 2H), 2.35 (m, 1H), 2.70 (s, 3H), 2.83 (m, 1H), 2.95 (m, 2H), 3.20 (t, J=7.1 Hz, 2H), 3.68 (m, 2H), 7.40 (m, 2H), 7.55 (dd, J=8.1, 18.6 Hz, 2H), 7.68 (d, J=22.6 Hz, 1H), 7.87 (d, J=8.4 Hz, 1H), 8.65 (s, 1H), 8.87 (s, 1H).

¹³C NMR (100 MHz, CDCl₃): δ 13.8, 18.7, 24.9, 34.9, 41.0, 45.3, 45.5, 46.2, 84.4, 122.1, 124.9, 125.7, 126.1, 127.3, 128.1, 130.2, 131.0, 132.4, 134.1, 137.5, 137.7, 142.7, 147.6, 148.0, 167.3.

### Synthesis 1d—Synthesis of [4-[4-(1-Cyanocyclopropyl)phenyl]-3-(4-methylsulfonylpiperazine-1-carbonyl)-8-quinolyl]boronic acid

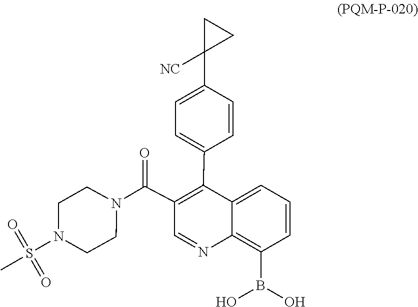 [SMILES: CS(=O)(=O)N1CCN(C(=O)c2cnc3c(B(O)O)cccc3c2-c2ccc(C3(C#N)CC3)cc2)CC1]

Following the procedure for PQM-P-001 using 2-chloroaniline (1 Eq) and DEEM (1.2 Eq) as starting reagents, formed residue was purified by C-18 reverse-phase flash column chromatography eluting with 0-100% Water/MeCN and the title compound was obtained as white solid.

¹H NMR (400 MHz, DMSO-d6): δ 1.64 (m, 2H), 1.84 (m, 2H), 2.18 (brs, 1H), 2.56 (m, 1H), 2.79 (s, 3H), 3.02 (m, 2H), 3.21 (m, 2H), 3.43 (m, 1H), 3.60 (brs, 1H), 7.51 (m, 4H), 7.69 (d, J=7.5 Hz, 1H), 7.77 (d, J=8.1 Hz, 1H), 8.34 (d, J=5.8 Hz, 1H), 8.97 (s, 1H), 9.48 (s, 1H).

¹³C NMR (100 MHz, DMSO-d6): δ 13.8, 19.2, 34.3, 41.0, 45.5, 45.7, 46.1, 119.4, 122.7, 125.8, 125.9, 128.2, 128.6, 129.1, 130.6, 130.9, 133.6, 137.7, 138.5, 144.9, 147.1, 151.8, 166.2.

### Synthesis 1e—Synthesis of 1-[4-[3-(4-Methylsulfonylpiperazine-1-carbonyl)-4-quinolyl]-2-(4,4,5,5-tetramethyl-1,3,2-dioxaborolan-2-yl)phenyl]cyclopropanecarbonitrile

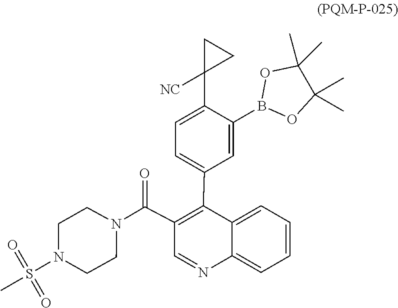

### Step 1—1-(2-Bromo-4-(4,4,5,5-tetramethyl-1,3,2-dioxaborolan-2-yl)phenyl)cyclopropane-1-carbonitrile

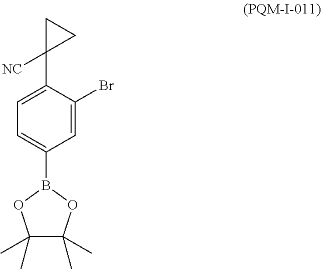 [SMILES: CC1(C)OB(c2ccc(C3(C#N)CC3)c(Br)c2)OC1(C)C]

Following the procedure for Synthesis 1a, steps 4 and 5 for PQM-1-007 replacing 2-(4-bromophenyl)acetonitrile of step 4 with 2-(2-bromo-4-iodophenyl)acetonitrile as starting reagent, formed residue was purified via flash chromatography eluting with 5-10% EtOAc n-hexane to afford the title compound as a white solid.

### Step 2—1-[4-[3-(4-Methylsulfonylpiperazine-1-carbonyl)-4-quinolyl]-2-(4,4,5,5-tetramethyl-1,3,2-dioxaborolan-2-yl)phenyl]cyclopropanecarbonitrile

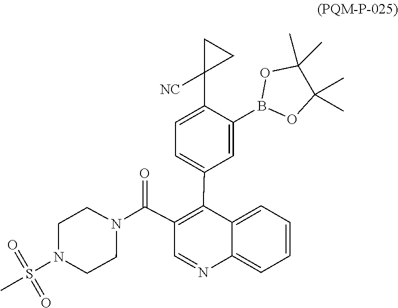

Following the procedure for Synthesis 1a, steps 1-9 for PQM-P-001 replacing 4-chloroaniline in step 1 with aniline and replacing 1-(4-(4,4,5,5-tetramethyl-1,3,2-dioxaborolan-2-yl)phenyl)cyclopropane-1-carbonitrile in step 6 with 1-(2-Bromo-4-(4,4,5,5-tetramethyl-1,3,2-dioxaborolan-2-yl)phenyl)cyclopropane-1-carbonitrile, the formed residue was purified by C-18 reverse-phase flash column chromatography eluting with 0-100% Water/MeCN to afford the title compound as a white solid.

¹H NMR (400 MHz, DMSO-d6): δ 1.38 (s, 12H), 1.45 (m, 1H), 1.65 (brs, 1H), 1.74 (m, 2H), 2.13 (m, 1H), 2.41 (m, 1H), 2.71 (s, 3H), 3.17 (m, 4H), 3.43 (d, J=11.2 Hz, 1H), 4.13 (d, J=13.2 Hz, 1H), 7.56 (m, 2H), 7.62 (d, J=8.0 Hz, 1H), 7.69 (d, J=8.2 Hz, 1H), 7.78 (m, 2H), 8.99 (d, J=8.2 Hz, 1H), 8.85 (s, 1H).

¹³C NMR (100 MHz, DMSO-d6): δ 14.7, 16.5, 17.7, 24.9, 25.0, 35.1, 41.1, 45.4, 45.6, 46.4, 84.7, 123.4, 125.9, 126.1, 127.8, 128.0, 129.9, 130.4, 130.5, 133.0, 134.5, 136.9, 142.6, 143.3, 147.3, 148.5, 167.4.

### Synthesis 1f—Synthesis of 1-[3-(4-Methylsulfonylpiperazine-1-carbonyl)-4-quinolyl]-4-[4-(4,4,5,5-tetramethyl-1,3,2-dioxaborolan-2-yl)phenyl]piperidine-4-carbonitrile

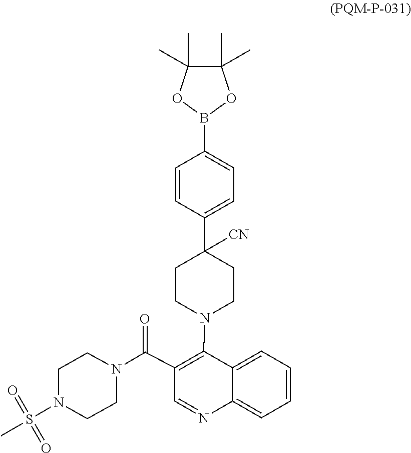 [SMILES: CC1(C)OB(c2ccc(C3(C#N)CCN(c4c(C(=O)N5CCN(S(C)(=O)=O)CC5)cnc5ccccc45)CC3)cc2)OC1(C)C]

### Step 1—Tert-butyl 4-(4-bromophenyl)-4-cyanopiperidine-1-carboxylate

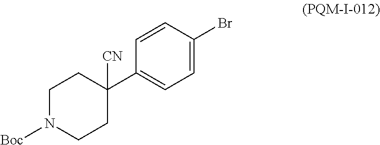 [SMILES: CC(C)(C)OC(=O)N1CCC(C#N)(c2ccc(Br)cc2)CC1]

100 mL round-bottomed flask charged with 2-(4-bromophenyl)acetonitrile (500 mg, 1 Eq, 2.55 mmol), tert-butyl bis(2-chloroethyl)carbamate (741 mg, 1.2 Eq, 3.06 mmol), NaH (245 mg, 4 Eq, 10.2 mmol), and 20 mL DMF at 0° C. Then mixture stirred at 60° C. for over night.

After consumption of starting compound (TLC monitoring) reaction was decreased 0° C. and quenched with slush. Following extracted with EtOAc (3×20 mL). The combined organic layers were washed with brine (3×20 ml), dried over Na2SO4, filtered and the solvent was eliminated under vacuo to give a residue that was purified by silica gel column chromatography eluting with 5-50% EtOAc/n-hexane to afford the title compound as a white solid with 52% yield.

### Step 2—4-(4-bromophenyl)piperidine-4-carbonitrile

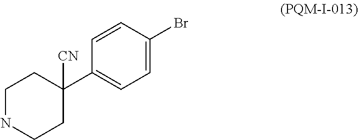 [SMILES: N#CC1(c2ccc(Br)cc2)CCNCC1]

100 mL round-bottomed flask charged with tert-butyl 4-(4-bromophenyl)-4-cyanopiperidine-1-carboxylate (200 mg, 1 Eq, 548 μmol), TFA (312 mg, 211 μL, 5 Eq, 2.74 mmol), and 20 mL DCM. Then mixture stirred at r.t. for 3 hrs. After consumption of starting compound (TLC monitoring) the solvent was eliminated under vacuo and crude diluted with 1M HCl solution in diethyl ether. Heavy precipitate was filtered and washed with cold diethyl ether (3×20 mL) to afford the title compound as a white solid with 69% yield and used as it is in the next step.

### Step 3—ethyl 4-(4-(4-bromophenyl)-4-cyanopiperidin-1-yl)quinoline-3-carboxylate

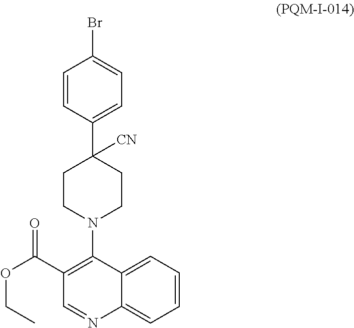 [SMILES: CCOC(=O)c1cnc2ccccc2c1N1CCC(C#N)(c2ccc(Br)cc2)CC1]

100 mL round-bottomed flask charged with 4-(4-bromophenyl)piperidine-4-carbonitrile (330 mg, 1 Eq, 1.24 mmol), commercially available ethyl 4-bromoquinoline-3-carboxylate (349 mg, 1 Eq, 1.24 mmol), DIPEA (804 mg, 1.08 mL, 5 Eq, 6.22 mmol) and 20 mL EtOH. Then mixture was refluxed for 3 hrs. After consumption of starting compound (TLC monitoring) the solvent was eliminated under vacuo. Formed residue was diluted with ethyl acetate (20 mL), rinsed with water (3×20 mL) following organic layer were washed with brine (3×10 ml), dried over Na2SO4, filtered and the solvent was eliminated under vacuo to give a residue that was purified silica gel column chromatography eluting with 5-60% EtOAC/n-hexane to afford corresponding compound as a white solid with 72% yield used as it is in the next step.

### Step 4—1-[3-(4-Methylsulfonylpiperazine-1-carbonyl)-4-quinolyl]-4-[4-(4,4,5,5-tetramethyl-1,3,2-dioxaborolan-2-yl)phenyl]piperidine-4-carbonitrile

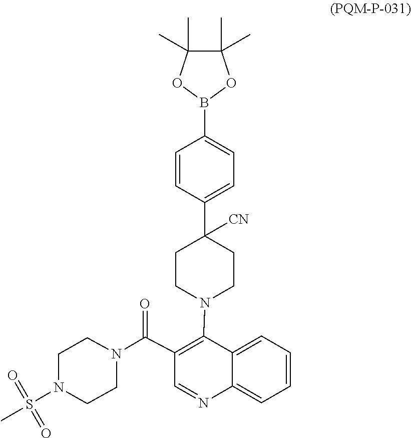 [SMILES: CC1(C)OB(c2ccc(C3(C#N)CCN(c4c(C(=O)N5CCN(S(C)(=O)=O)CC5)cnc5ccccc45)CC3)cc2)OC1(C)C]

Following the procedure for Synthesis 1a, steps 7-9 for PQM-P-001, replacing PQM-1-008 in step 7 with PQM-1-014, formed residue was purified by C-18 reversed phase flash column chromatography eluting with 0-100% Water/MeCN and the title compound was obtained as white solid 45% yield.

¹H NMR (400 MHz, DMSO-d6): δ 1.31 (s, 12H), 2.33 (m, 3H), 2.93 (s, 3H), 3.17 (m, 2H), 3.30 (m, 2H), 3.34 (m, 3H), 3.44 (m, 1H), 3.53 (m, 1H), 3.65 (d, J=12.6 Hz, 1H), 3.75 (m, 1H), 3.93 (m, 1H), 7.65 (d, J=7.3 Hz, 1H), 7.70 (d, J=8.3 Hz, 2H), 7.79 (m, 3H), 8.01 (d, J=8.0 Hz, 1H), 8.22 (d, J=8.0 Hz, 1H), 8.60 (s, 1H).

¹³C NMR (100 MHz, DMSO-d6): δ 25.1, 34.9, 36.1, 36.4, 41.4, 42.9, 45.6, 45.7, 47.1, 48.6, 50.3, 84.3, 121.6, 122.1, 124.6, 125.1, 125.8, 127.1, 129.9, 130.5, 135.6, 143.6, 149.5, 153.1, 167.6

### Synthesis 2—Synthesis of Reference “Cold” Compounds (PQM-C Compounds)

### General Procedure A—Fluorination of PQM-P Compounds

A 10 mL round bottom flask was charged with sodium hydrate (1.2 Eq), corresponding quinoline boronic ester or quinoline boronic acid (PQM-P compound) (0.170 μM, 1 Eq) and 5 mL MeOH. After stirring for 15 min at r.t, the reaction mixture was cooled to 0° C. and silver(I)trifluoromethane sulfonate (3 Eq) was added. After stirring for 30 min at 0° C., the solvent was removed under reduced pressure at 0° C. and the residual MeOH was completely removed by co-evaporation with acetone (2.5 mL×2). To the residue acetone (5.0 mL), MS3 Å (250 mg), and 1-(chloromethyl)-4-fluoro-1,4-diazabicyclo[2.2.2]octane-1,4-diium tetrafluoroborate (1 Eq) were added. The reaction mixture was stirred for an hour and concentrated in vacuo. The residue was dissolved in DCM and filtered through celite. After removal of DCM, the residue was diluted with ethyl acetate (20 mL), rinsed with water (3×20 mL) and the organic layer was washed with brine (3×10 ml), dried over Na₂SO₄, filtered and the solvent was removed in vacuo to give a residue that was purified by C-18 reversed phase flash column chromatography eluting with 0-100% Water/MeCN to afford corresponding compound as a white solid.

### General Procedure B—Iodination of PQM-P Compounds

A 0.5 mL Borax tube was charged with corresponding quinoline boronic ester or quinoline boronic acid (150 μm, 1 Eq) (PQM-P compound), cuprous iodide (0.05 Eq), N-iodosuccinimide (1.3 Eq), Iodine (1 Eq) and 0.2 mL ACN. The reaction mixture was heated in a closed system via a microwave synthesizer (CEM Discovery SP) at 80° C. for 10 min. Following completion, the vessel was cooled by air jet cooling. All solvent was removed in vacuo to give a residue that was purified by C-18 reversed phase flash column chromatography, eluting with 0-100% Water/ACN to afford corresponding compound as a white solid.

### Synthesis 2a—Synthesis of 1-(4-(6-fluoro-3-(4-(methylsulfonyl)piperazine-1-carbonyl)quinolin-4-yl)phenyl)cyclopropane-1-carbonitrile

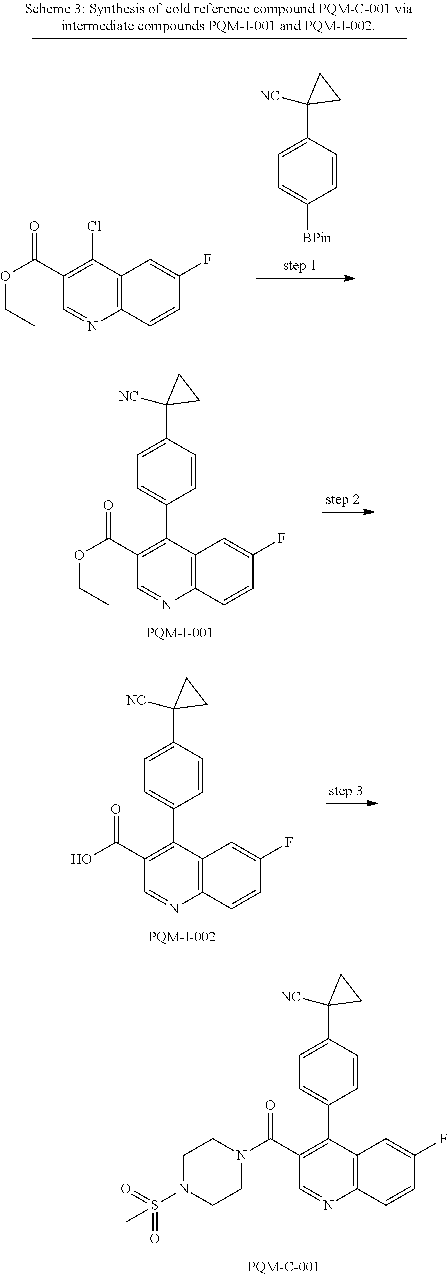

### Step 1—Ethyl 4-(4-(1-cyanocyclopropyl)phenyl)-6-fluoroquinoline-3-carboxylate

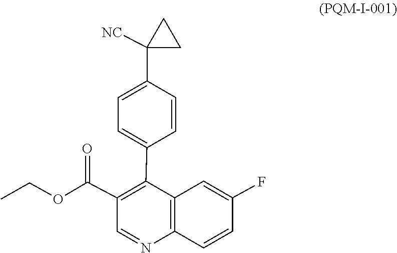 [SMILES: CCOC(=O)c1cnc2ccc(F)cc2c1-c1ccc(C2(C#N)CC2)cc1]

An oven-dried round-bottom flask (25 mL) was charged with ethyl 4-chloro-6-fluoroquinoline-3-carboxylate, 1-(4-(4,4,5,5-tetramethyl-1,3,2-dioxaborolan-2-yl)phenyl) cyclopropane-1-carbonitrile, PdCl₂(dppf)-DCM, K₂CO₃. The flask was evacuated and refilled with nitrogen (×3) after which DMF was injected and set to stir at 110° C. for 1.5 h. The reaction was cooled to room temperature, diluted with ethyl acetate (50 mL), and then washed with water (40 mL×2) and brine (40 mL) before being dried over MgSO₄. The organic solvents were removed under reduced pressure to yield a dark brown solid. Purification via flash chromatography [silica gel (24 g), ethyl acetate/petroleum ether (20:80) to (50:50)] to yield ethyl 4-(4-(1-cyanocyclopropyl)phenyl)-6-fluoroquinoline-3-carboxylate as a white solid (0.44 g, 66%).

¹H NMR (400 MHz, CDCl₃): δ[ppm]=1.05 (t, J=7.1 Hz, 3H, CH₃), 1.55 (m, 2H, CH₂), 1.84 (m, 2H, CH₂), 4.15 (q, J=7.2 Hz, 2H, CH₂), 7.28 (d, J=8.2 Hz, 2H, Ar—H), 7.45 (d, J=8.2 Hz, 2H, Ar—H), 7.49 (d, J=2.3 Hz, 1H, Ar—H), 7.73 (dd J=9.0, 2.3 Hz, 1H, Ar—H) 8.14 (d, J=9.0 Hz, 1H, Ar—H), 9.33 (s, 1H, Ar—H).

¹³C NMR (100 MHz, CDCl₃): δ[ppm]=13.86, 13.99, 18.83, 61.7, 122.4, 124.1, 125.7, 126.0, 127.9, 129.5, 131.3, 132.3, 133.8, 135.3, 136.7, 147.5, 148.3, 150.3, 166.0.

### Step 2—4-(4-(1-cyanocyclopropyl)phenyl)-6-fluoroquinoline-3-carboxylic acid

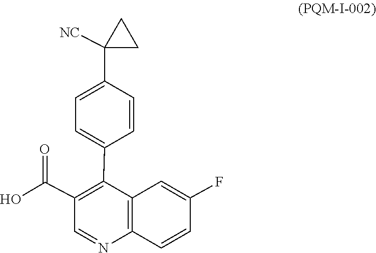 [SMILES: N#CC1(c2ccc(-c3c(C(=O)O)cnc4ccc(F)cc34)cc2)CC1]

A round-bottom flask (25 mL) was charged with ethyl 4-chloro-6-fluoroquinoline-3-carboxylate, NaOH (1N, aq.), THF and MeOH and set to stir vigorously at 50° C. for 4 hours. Then, the reaction mixture was cooled to 0° C., and the pH was adjusted to appx. 4-5 with the aid of HCl (1N, aq.). The solvents were removed under reduced pressure to give a yellow solid which was triturated with small amount of water to give 4-(4-(1-cyanocyclopropyl)phenyl)-6-fluoroquinoline-3-carboxylic acid as white solid (0.32 g, 87%).

### Step 3—1-(4-(6-fluoro-3-(4-(methylsulfonyl)piperazine-1-carbonyl)quinolin-4-yl)phenyl)cyclopropane-1-carbonitrile

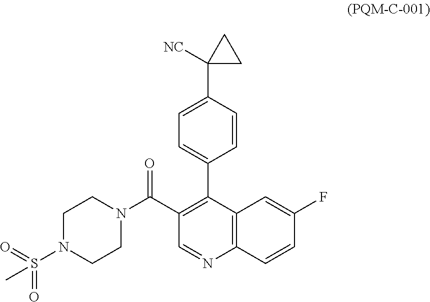 [SMILES: CS(=O)(=O)N1CCN(C(=O)c2cnc3ccc(F)cc3c2-c2ccc(C3(C#N)CC3)cc2)CC1]

A round-bottom flask (25 mL) was charged with 4-(4-(1-cyanocyclopropyl)phenyl)-6-fluoroquinoline-3-carboxylic acid, HATU, DIPEA, and DMF. Set to stir at room temp for 15 min after which 1-(methylsulfonyl)piperazine was added and the reaction was stirred overnight at room temperature. The volatile solvents were removed under reduced pressure (rotovap), and then the resultant liquid was diluted with ethyl acetate (75 mL) after which it was rinsed with water (30 mL×2), brine (30 mL). The ethyl acetate phase was dried over MgSO₄ and the volatiles were removed under reduced pressure to furnish an orange oil which was purified via flash chromatography [Buchi-silica gel (24 g), DCM wet-load, methylene chloride (0.5% Et₃N)/ethyl acetate (0.5% Et₃N) (80:20) to (20:80)] to yield 1-(4-(6-fluoro-3-(4-(methylsulfonyl)piperazine-1-carbonyl)quinolin-4-yl)phenyl)cyclopropane-1-carbonitrile as pale yellow solid (0.401 g, 46% yield).

¹H NMR (400 MHz, CDCl3) δ 1.53 (m, 2H, CH2), 1.85 (m, 2H, CH2), 2.37 (m, 1H), 2.71 (s, 3H, CH3), 2.81 (m, 1H), 2.98 (m, 2H), 3.22 (m, 2H), 3.70 (s, 2H), 7.34 (d, J=8.0 Hz, 1H, Ar—H), 7.41 (m, 2H, Ar—H) 7.57 (m, 3H, Ar—H), 8.20 (m, 1H, Ar—H), 8.84 (s, 1H, Ar—H).

### Synthesis 2b—Synthesis of 1-(4-(6-Fluoro-3-(4-(methylsulfonyl)piperazine-1-carbonyl)-8-nitroquinolin-4-yl)phenyl)cyclopropane-1-carbonitrile

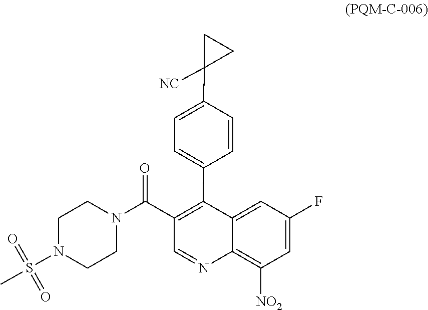 [SMILES: CS(=O)(=O)N1CCN(C(=O)c2cnc3c([N+](=O)[O-])cc(F)cc3c2-c2ccc(C3(C#N)CC3)cc2)CC1]

Following the General Procedure A using PQM-P-007 as starting material, formed residue was purified by C-18 reverse phase flash column chromatography eluting with 0-100% Water/MeCN and the title compound was obtained as white solid.

¹H NMR (400 MHz, DMSO-d6): δ 1.54 (m, 2H), 1.85 (m, 2H), 2.37 (m, 1H), 2.71 (s, 3H), 2.82 (m, 1H), 2.97 (m, 2H), 3.22 (m, 2H), 3.69 (t, J=4.8 Hz 2H), 7.39 (d, J=8.0 Hz, 1H), 7.45 (d, J=7.9 Hz, 1H) 7.55 (brs, 2H), 7.69 (d, J=1.9 Hz), 7.74 (dd, J=9.0, 2.3 Hz, 1H), 8.13 (d, J=9.0 Hz, 1H), 8.85 (s, 1H).

¹³C NMR (100 MHz, DMSO-d6): 13.9, 18.8, 35.2, 41.3, 45.5, 45.7, 46.4, 122.1, 124.8, 126.5, 126.6, 128.5, 131.1, 131.5, 131.7, 133.5, 134.1, 137.9, 142.5, 147.0, 147.9, 166.9.

¹⁹F NMR (376 MHz, DMSO-d6): δ-114.4.

### Synthesis 2c—Synthesis of 1-(4-(6-Iodo-3-(4-(methylsulfonyl)piperazine-1-carbonyl)-8-nitroquinolin-4-yl)phenyl)cyclopropane-1-carbonitrile

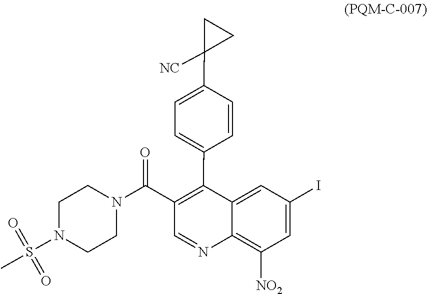 [SMILES: CS(=O)(=O)N1CCN(C(=O)c2cnc3c([N+](=O)[O-])cc(I)cc3c2-c2ccc(C3(C#N)CC3)cc2)CC1]

Following the General Procedure B using PQM-P-007 as starting material, formed residue was purified by C-18 reverse phase flash column chromatography eluting with 0-100% Water/MeCN and the title compound was obtained as white solid.

¹H NMR (400 MHz, DMSO-d6): δ 1.68 (m, 2H), 1.85 (m, 2H), 2.15 (t, J=8.9, 1H), 2.56 (t, J=9.9, 1H), 2.79 (s, 3H), 3.04 (m, 2H), 3.18 (m, 2H), 3.45 (m, 1H), 3.60 (m, 1H), 7.53 (m, 4H), 8.16 (s, 1H), 8.73 (s, 1H), 9.03 (s, 1H).

¹³C NMR (100 MHz, DMSO-d6): 13.8, 19.4, 34.4, 45.5, 45.7, 46.0, 92.6, 122.6, 126.0, 128.8, 130.8, 131.0, 131.5, 132.3, 137.9, 138.1, 138.4, 142.7, 148.9, 150.6, 165.5.

### Synthesis 2d—Synthesis of 1-(4-(7-Fluoro-3-(4-(methylsulfonyl)piperazine-1-carbonyl)quinolin-4-yl)phenyl)cyclopropane-1-carbonitrile

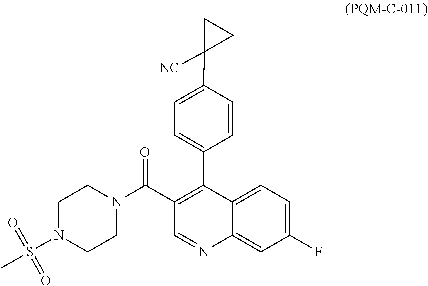 [SMILES: CS(=O)(=O)N1CCN(C(=O)c2cnc3cc(F)ccc3c2-c2ccc(C3(C#N)CC3)cc2)CC1]

Following the General Procedure A using PQM-P-013 as starting material, formed residue was purified by C-18 reverse phase flash column chromatography eluting with 0-100% Water/MeCN and the title compound was obtained as white solid.

¹H NMR (400 MHz, DMSO-d6): δ 1.63 (m, 2H), 1.83 (m, 2H), 2.15 (m, 1H), 2.57 (m, 1H), 2.78 (s, 3H), 3.02 (m, 2H), 3.21 (m, 2H), 3.45 (brs, 1H), 3.60 (brs, 1H), 7.40 (d, J=7.6 Hz, 1H), 7.49 (d, J=7.6 Hz, 1H), 7.58 (m, 3H), 7.74 (dd, J=6.1, 9.1 Hz, 1H), 7.90 (dd, J=2.6, 10.1 Hz, 1H), 8.91 (s, 1H).

¹⁹F NMR (376 MHz, DMSO-d6): δ-109.4.

### Synthesis 2e—Synthesis of 1-(4-(7-Iodo-3-(4-(methylsulfonyl)piperazine-1-carbonyl)quinolin-4-yl)phenyl)cyclopropane-1-carbonitrile

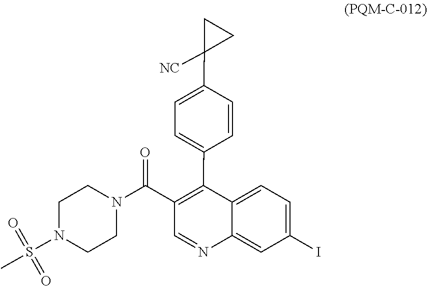 [SMILES: CS(=O)(=O)N1CCN(C(=O)c2cnc3cc(I)ccc3c2-c2ccc(C3(C#N)CC3)cc2)CC1]

Following the General Procedure B using PQM-P-013 as starting material, formed residue was purified by C-18 reverse phase flash column chromatography eluting with 0-100% Water/MeCN and the title compound was obtained as white solid.

¹H NMR (400 MHz, DMSO-d6): δ 1.64 (m, 2H), 1.83 (m, 2H), 2.12 (m, 1H), 2.55 (m, 1H), 2.78 (s, 3H), 2.99 (m, 2H), 3.21 (brs, 2H), 3.43 (m, 1H), 3.60 (brs, 1H), 7.43 (d, J=8.8 Hz, 1H), 7.53 (m, 3H), 7.74 (d, J=8.9 Hz, 1H), 8.56 (s, 1H), 8.88 (s, 1H).

¹³C NMR (100 MHz, DMSO-d6): δ 13.8, 19.2, 34.3, 45.5, 45.7, 46.1, 61.2, 97.9, 122.7, 125.3, 125.9, 128.1, 129.2, 130.7, 130.9, 133.3, 136.7, 137.8, 138.1, 143.5, 148.9, 149.2, 166.4.

### Synthesis 2f—Synthesis of 1-(4-(8-Fluoro-3-(4-(methylsulfonyl)piperazine-1-carbonyl)quinolin-4-yl)phenyl)cyclopropane-1-carbonitrile

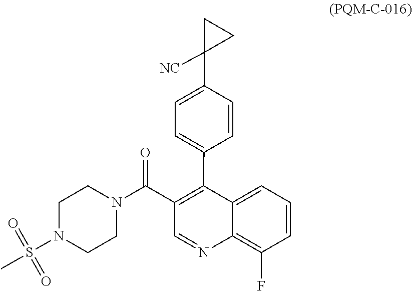 [SMILES: CS(=O)(=O)N1CCN(C(=O)c2cnc3c(F)cccc3c2-c2ccc(C3(C#N)CC3)cc2)CC1]

Following the procedure for Synthesis 1a, steps 1-8 for PQM-1-010 replacing 4-chloroaniline in step 1 with 2-fluoroaniline (1 Eq) and DEEM (1.2 Eq) as starting reagents, formed residue was purified by C-18 reverse phase flash column chromatography eluting with 0-100% Water/MeCN and the title compound was obtained as white solid.

¹H NMR (400 MHz, DMSO-d6): δ 1.63 (m, 2H), 1.83 (m, 2H), 2.17 (t, J=8.3 Hz, 1H), 2.57 (t, J=8.2 Hz, 1H), 2.79 (s, 3H), 3.01 (m, 1H), 3.05 (m, 1H), 3.21 (m, 2H), 3.47 (brs, 1H), 3.59 (brs, 1H), 7.41 (d, J=7.6 Hz, 1H), 7.48 (d, J=8.5 Hz, 1H), 7.59 (m, 3H), 7.71 (m, 1H), 8.94 (s, 1H).

¹³C NMR (100 MHz, DMSO-d6): δ 13.8, 19.2, 34.3, 40.9, 45.5, 45.7, 46.1, 114.8, 122.6, 125.8, 128.0, 128.2, 129.7, 130.6, 133.4, 137.8, 138.3, 143.2, 148.0, 148.5, 156.6, 166.2. ¹⁹F NMR (376 MHz, DMSO-d6): δ −124.8.

### Synthesis 2q—Synthesis of 1-(4-(8-Iodo-3-(4-(methylsulfonyl)piperazine-1-carbonyl)quinolin-4-yl)phenyl)cyclopropane-1-carbonitrile

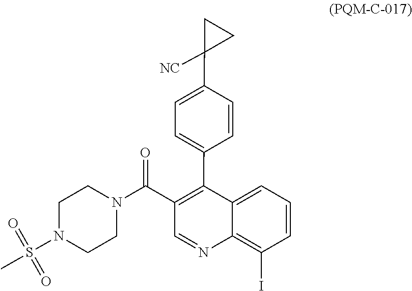 [SMILES: CS(=O)(=O)N1CCN(C(=O)c2cnc3c(I)cccc3c2-c2ccc(C3(C#N)CC3)cc2)CC1]

Following the General Procedure B using PQM-P-020 as starting material, formed residue was purified by C-18 reverse phase flash column chromatography eluting with 0-100% Water/MeCN and the title compound was obtained as white solid.

¹H NMR (400 MHz, DMSO-d6): δ 1.63 (m, 2H), 1.83 (m, 2H), 2.21 (m, 1H), 2.57 (m, 1H), 2.79 (s, 3H), 3.01 (m, 2H), 3.22 (m, 2H), 3.49 (m, 1H), 3.58 (m, 1H), 7.38 (m, 2H), 7.50 (m, 2H), 7.57 (m, 1H), 7.67 (d, J=8.0 Hz, 1H), 8.49 (d, J=7.3 Hz, 1H), 8.95 (s, 1H).

¹³C NMR (100 MHz, DMSO-d6): δ 13.8, 19.2, 34.3, 41.0, 45.5, 45.8, 46.1, 104.8, 122.7, 125.8, 127.1, 127.4, 129.6, 129.7, 130.7, 130.9, 133.3, 137.7, 141.1, 144.2, 146.7, 149.2, 166.2.

### Synthesis 2h—Synthesis of 1-(4-(3-(4-(methylsulfonyl)piperazine-1-carbonyl)quinolin-4-yl)-2-(4,4,5,5-tetramethyl-1,3,2-dioxaborolan-2-yl)phenyl)cyclopropane-1-carbonitrile

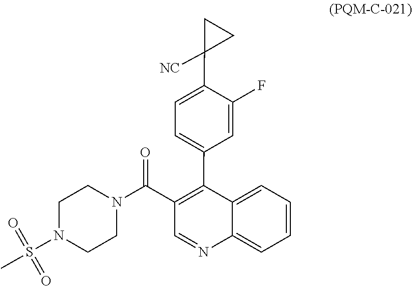 [SMILES: CS(=O)(=O)N1CCN(C(=O)c2cnc3ccccc3c2-c2ccc(C3(C#N)CC3)c(F)c2)CC1]

Following the General Procedure A using PQM-P-025 as starting material, formed residue was purified by C-18 reverse phase flash column chromatography eluting with 0-100% Water/MeCN and the title compound was obtained as white solid.

¹H NMR (400 MHz, DMSO-d6): δ 1.57 (m, 2H), 1.77 (m, 2H), 2.44 (m, 1H), 2.66 (m, 1H), 2.82 (s, 3H), 3.04 (m, 2H), 3.20 (brs, 2H), 3.52 (brs, 1H), 4.13 (d, J=13.2 Hz, 1H), 7.31 (m, 1H), 7.53 (m, 1H), 7.66 (m, 3H), 7.86 (m, 1H), 8.16 (d, J=8.5 Hz, 1H), 8.91 (s, 1H).

¹⁹F NMR (376 MHz, DMSO-d6): δ −108.8

### Synthesis 2i—Synthesis of 1-(2-Iodo-4-(3-(4-(methylsulfonyl)piperazine-1-carbonyl)quinolin-4-yl)phenyl)cyclopropane-1-carbonitrile

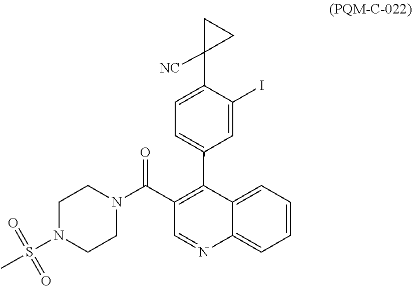 [SMILES: CS(=O)(=O)N1CCN(C(=O)c2cnc3ccccc3c2-c2ccc(C3(C#N)CC3)c(I)c2)CC1]

Following the General Procedure B using PQM-P-025 as starting material, formed residue was purified by C-18 reverse phase flash column chromatography eluting with 0-100% Water/MeCN and the title compound was obtained as yellow solid.

¹H NMR (400 MHz, DMSO-d6): δ 1.57 (m, 2H), 1.77 (m, 2H), 2.44 (m, 1H), 2.66 (m, 1H), 2.82 (s, 3H), 3.04 (m, 2H), 3.20 (brs, 2H), 3.52 (brs, 1H), 4.13 (d, J=13.2 Hz, 1H), 7.31 (m, 1H), 7.53 (m, 1H), 7.66 (m, 3H), 7.86 (m, 1H), 8.16 (d, J=8.5 Hz, 1H), 8.91 (s, 1H).

¹³C NMR (100 MHz, DMSO-d6): δ 14.7, 16.5, 17.7, 24.9, 25.0, 35.1, 41.1, 45.4, 45.6, 46.4, 84.7, 123.4, 125.9, 126.1, 127.8, 128.0, 129.9, 130.4, 130.5, 133.0, 134.5, 136.9, 142.6, 143.3, 147.3, 148.5, 167.4

### Synthesis 2j—Synthesis of 4-(4-fluorophenyl)-1-(3-(4-(methylsulfonyl)piperazine-1-carbonyl)quinolin-4-yl)piperidine-4-carbonitrile

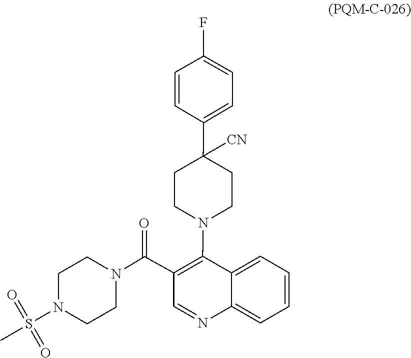 [SMILES: CS(=O)(=O)N1CCN(C(=O)c2cnc3ccccc3c2N2CCC(C#N)(c3ccc(F)cc3)CC2)CC1]

Following the General Procedure A using PQM-P-031 as starting material, formed residue was purified by C-18 reverse phase flash column chromatography eluting with 0-100% Water/MeCN and the title compound was obtained as white solid.

¹H NMR (400 MHz, DMSO-d6): δ 2.24 (m, 4H), 2.41 (m, 2H), 2.921 (s, 3H), 3.14 (m, 3H), 3.49 (m, 6H), 3.77 (m, 2H), 3.89 (m, 1H), 6.85 (d, J=8.7 Hz, 2H), 7.45 (d, J=8.7 Hz, 2H), 7.67 (t, J=8.0 Hz, 1H), 7.84 (t, J=8.0 Hz, 1H), 8.01 (d, J=8.3 Hz, 1H), 8.21 (d, J=8.6 Hz, 1H), 8.66 (s, 1H).

¹⁹F NMR (376 MHz, DMSO-d6): δ −148.2

### Synthesis 2k—Synthesis of 4-(4-iodophenyl)-1-(3-(4-(methylsulfonyl)piperazine-1-carbonyl)quinolin-4-yl)piperidine-4-carbonitrile

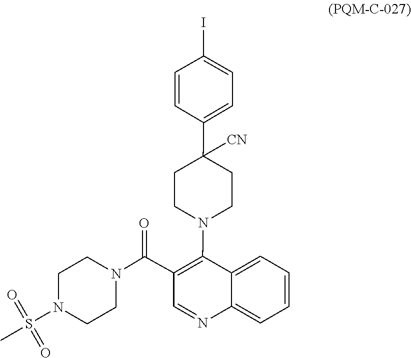 [SMILES: CS(=O)(=O)N1CCN(C(=O)c2cnc3ccccc3c2N2CCC(C#N)(c3ccc(I)cc3)CC2)CC1]

Following the General Procedure B using PQM-P-031 as starting material, formed residue was purified by C-18 reverse phase flash column chromatography eluting with 0-100% Water/MeCN and the title compound was obtained as white solid.

¹H NMR (400 MHz, DMSO-d6): δ 2.24 (m, 4H), 2.41 (m, 2H), 2.921 (s, 3H), 3.14 (m, 3H), 3.49 (m, 6H), 3.77 (m, 2H), 3.89 (m, 1H), 6.85 (d, J=8.7 Hz, 2H), 7.45 (d, J=8.7 Hz, 2H), 7.67 (t, J=8.0 Hz, 1H), 7.84 (t, J=8.0 Hz, 1H), 8.01 (d, J=8.3 Hz, 1H), 8.21 (d, J=8.6 Hz, 1H), 8.66 (s, 1H).

### Synthesis 3—Synthesis of PET/SPECT Radiotracers (PQM-H Compounds)

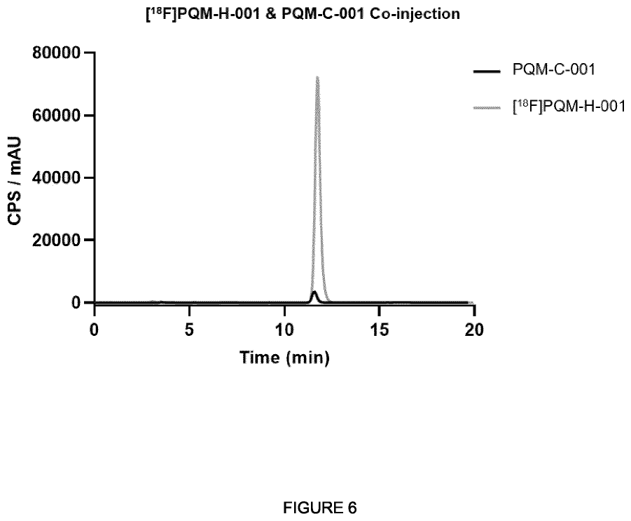

### Synthesis 3a—Synthesis of 1-(4-(6-(fluoro-18F)-3-(4-(methylsulfonyl)piperazine-1-carbonyl)quinolin-4 yl)phenyl)cycloPropane-1-carbonitrile (Scheme 2, Step 10A)

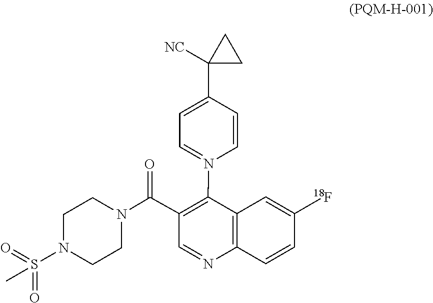

[¹⁸F]Fluoride (900 MBq) in ¹⁸O-water was trapped on a Sep-Pak® QMA cartridge, released with a solution (0.65 mL) of Kryptofix 222 and potassium carbonate solution to a V-Wheaton vial. After removing the solvent by heating at 110° C. under a stream of nitrogen presence of dry acetonitrile (3×0.1 mL), the vial was charged with 300 μL DMA solution containing 1-[4-[3-(4-methylsulfonylpiperazine-1-carbonyl)-6-(4,4,5,5-tetramethyl-1,3,2-dioxaborolan-2-yl)-4-quinolyl]phenyl]cyclopropanecarbonitrile (PQM-P-001) (11.7 mg, 1 Eq., 0.0200 mmol) and Tetrakis(pyridine)copper(II) bis(trifluoromethanesulfonate) (33.9 mg, 2.5 Eq., 50.0 μmol). Reaction was mixed for 20 min at 110° C. Then, the reaction was quenched with 100 μL water and the crude mixture was diluted with 15 mL of demineralized water and passed thorough pre-treated (5 mL EtOH 10 mL water) Sep-Pak® HLB cartridge. Trapped labelled compound was released from cartridge with EtOH and purified via semi preparative HPLC Method 3 (Rt≈11 min). The isolated fraction was diluted and passed through a Sep-Pak® HLB cartridge, then pure labelled compound was eluted with ethanol (4 mL). Ethanol was removed under a flow of nitrogen and the residue was reformulated in PBS buffer which was passed through a sterile filter into a sealed sterile vial. The decay corrected isolated RCY was calculated as 10% at the end of HPLC purification with radiochemical purity of >99%. Radiochemical product was confirmed by co-elution with the non-radioactive analogue (FIG. 6).

### Synthesis 3b—Synthesis of 1-(4-(6-(iodo-125I)-3-(4-(methylsulfonyl)piperazine-1-carbonyl)quinolin-4-yl)phenyl)cyclopropane-1-carbonitrile (Scheme 2, Step 10B)

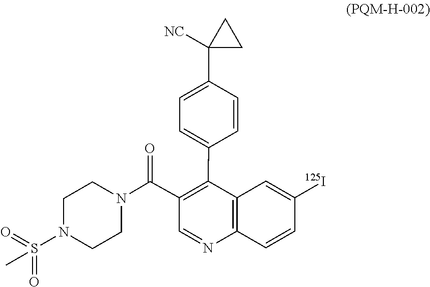 [SMILES: CS(=O)(=O)N1CCN(C(=O)c2cnc3ccc([125I])cc3c2-c2ccc(C3(C#N)CC3)cc2)CC1]

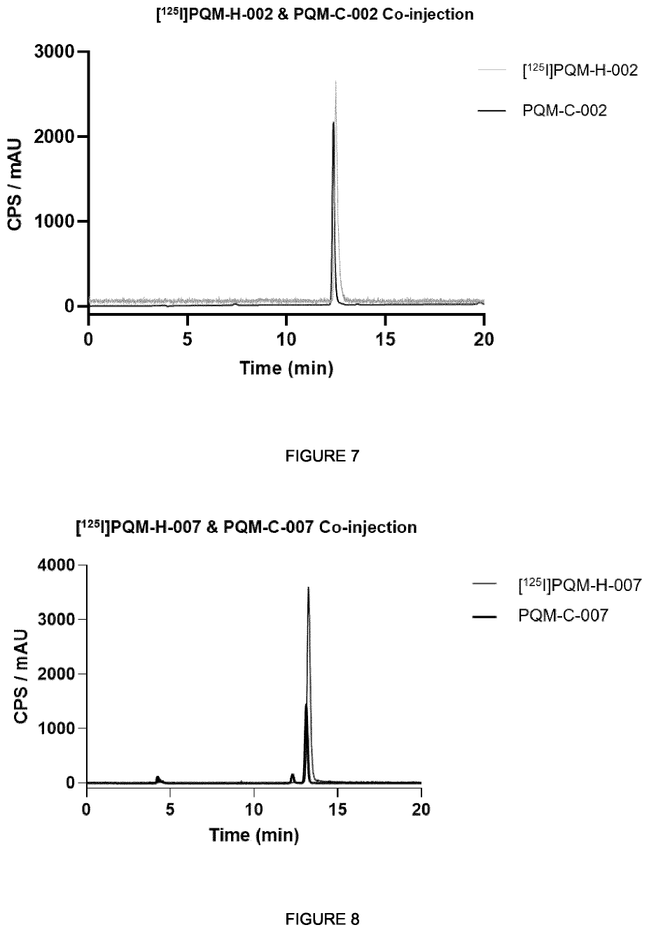

A V-vial was charged with, 1-[4-[3-(4-methylsulfonylpiperazine-1-carbonyl)-6-(4,4,5,5-tetramethyl-1,3,2-dioxaborolan-2-yl)-4-quinolyl]phenyl]cyclopropanecarbonitrile (PQM-P-001) (2.9 mg, 1 Eq., 0.005 mmol), [123I]NaI (ca. 7.5 KBq, ~2 μL MeOH) and Cu(OCOCF₃)₂ (0.25 mol) and 1,10-phenanthroline (0.25 μmol) in MeOH:H₂O (4:1, 200 μL). The reaction vial was then heated to 80° C. for 20 minutes. After, the reaction was diluted in H₂O (10 mL) and loaded onto a Sep-Pak® HLB cartridge. Trapped labelled compound was released from cartridge with EtOH and purified via semi-preparative HPLC Method 3 (R_{T}≈12 min). The isolated fraction was diluted and passed through a Sep-Pak® HLB cartridge, then pure labelled compound was eluted with ethanol (4 mL). Ethanol was removed under a flow of nitrogen and the residue was reformulated in PBS buffer which was passed through a sterile filter into a sealed sterile vial. RCY was calculated as was 40% at the end of HPLC purification with radiochemical purity of >99%. Radiochemical product was confirmed by co-elution with the non-radioactive analogue (FIG. 7).

### Synthesis 3c—Synthesis of 1-(4-(6-(Iodo-¹²⁵I)-3-(4-(methylsulfonyl)piperazine-1-carbonyl)-8-nitroquinolin-4-yl)phenyl)cyclopropane-1-carbonitrile

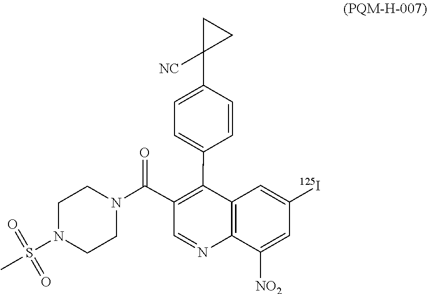 [SMILES: CS(=O)(=O)N1CCN(C(=O)c2cnc3c([N+](=O)[O-])cc([125I])cc3c2-c2ccc(C3(C#N)CC3)cc2)CC1]

Following the procedure for PQM-H-002 using PQM-P-007 as starting material, the formed residue was reformulated in PBS buffer which was passed through a sterile filter into a sealed sterile vial. RCY was calculated as 30% at the end of HPLC purification with radiochemical purity of >99%. Radiochemical product was confirmed by co-elution with the non-radioactive analogue (FIG. 8).

### Synthesis 3d—Synthesis of 1-(4-(8-(Iodo-125I)-3-(4-(methylsulfonyl)piperazine-1-carbonyl)quinolin-4-yl)phenyl)cyclopropane-1-carbonitrile

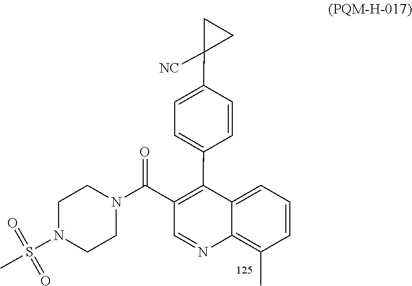 [SMILES: Cc1cccc2c(-c3ccc(C4(C#N)CC4)cc3)c(C(=O)N3CCN(S(C)(=O)=O)CC3)cnc12]

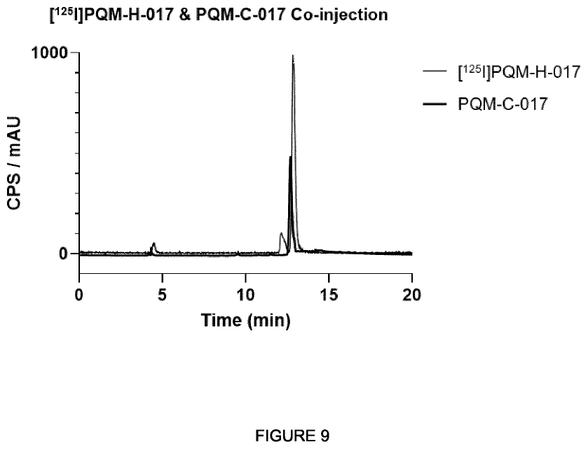

Following the procedure for PQM-H-002 using PQM-P-020 as starting material, formed residue was reformulated in PBS buffer which was passed through a sterile filter into a sealed sterile vial. RCY was calculated as 35% at the end of HPLC purification with radiochemical purity of >99%. Radiochemical product was confirmed by co-elution with the non-radioactive analogue (FIG. 9).

### Synthesis 3d—Synthesis of 1-(2-(Iodo-125I)-4-(3-(4-(methylsulfonyl)piperazine-1-carbonyl) uinoline-4-yl)phenyl)cyclopropane-1-carbonitrile

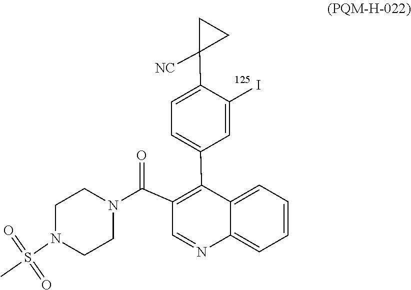 [SMILES: CS(=O)(=O)N1CCN(C(=O)c2cnc3ccccc3c2-c2ccc(C3(C#N)CC3)c(I)c2)CC1]

Following the procedure for PQM-H-002 using PQM-P-025 as starting material, formed residue was reformulated in PBS buffer which was passed through a sterile filter into a sealed sterile vial. RCY was calculated as was 10% at the end of HPLC purification with radiochemical purity of >99%. Radiochemical product was confirmed by co-elution with the non-radioactive analogue.

### BIOLOGICAL EXAMPLES

### Target Characterization

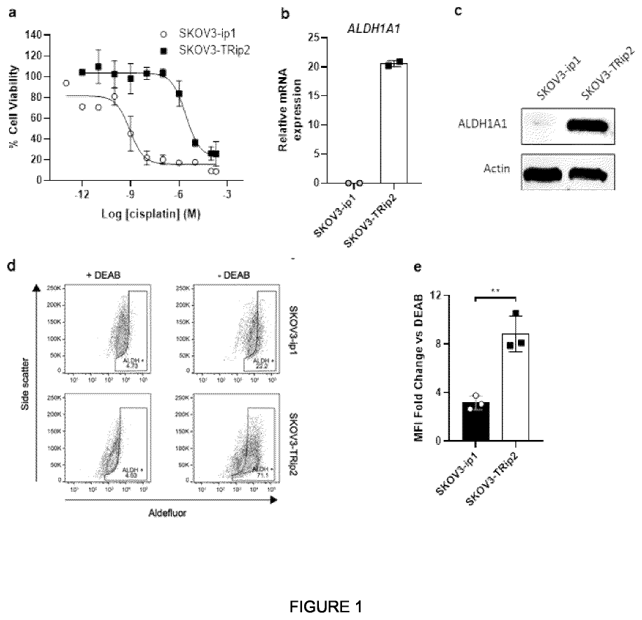

Human SKOV3 ovarian cancer cells were used as a tumour model. Two variants of this cell line exist which are either sensitive to therapy (SKOV3-ip1) and have low ALDH1A1 activity or are resistant (SKOV-TRip2) and present with high ALDH1A1 activity (FIG. 1).

The SKOV3-TRip2 cell line was generated through progressive exposure to paclitaxel (Duan et al., 1999) whereas the SKOV3-ip1 cell line remained untreated. Similar to previous findings with paxlitaxel (Landen et al., 2010), resistance to cisplatin was increased by over three orders of magnitude in SKOV3-TRip2 cells compared to SKOV3-ip1 (EC50, 0.88 nM vs. 2.6 μM, respectively; n=3-5; FIG. 1a). In SKOV3-TRip2 cells, ALDH1A1 mRNA, FIG. 1b, and protein expression, FIG. 1c, were substantially increased in comparison to SKOV3-ip1 cells. ALDH activity, as measured by the Aldefluor assay (Pereira, R., et al., 2022), was also significantly increased in SKOV3-TRip2 cells compared to SKOV3-ip1 (n=3; P<0.01; FIG. 1d, 1e).

### Aldeflour Assay

ALDH activity was measured by flow cytometry using the ALDEFLUOR kit (STEMCELL Technologies 01700) according to manufacturer's instructions. Cells were ALDEFLUOR stained at a concentration of 5×10⁵ cells/mL for 45 minutes at 37° C. in the presence or absence of 60 μM DEAB. The cell suspension was passed through a 35 μM filter and kept on ice prior to analysis. Flow cytometry was performed on a BD FACSMelody using FACS Chorus software (488 nm laser and 527/32 bandpass filter), followed by analysis with FlowJo. A minimum of 10,000 events were collected for each sample. Data were gated post-acquisition based on forward (FS) and side scattering (SS) profiles to include only single cell events and to exclude cellular debris.

mRNA Expression

RNA was extracted from SKOV3-ip1 and SKOV3-TRip2 cells using the RNeasy Kit (Qiagen, UK) and cDNA was synthesised using a Quantitect Reverse Transcription kit (Qiagen, UK) as per the manufacturer's instructions. A Rotor-Gene SYBR Green (Qiagen, UK) master mix was prepared according to the manufacturer's instructions using Quantitect Primer Assay for human ACTB and ALDH1A1 (Qiagen, UK). Ct values were obtained using Rotor-Gene Q software. Change in expression was measured using the DDCt method and expressed as relative expression versus the experimental control or an internal universal reference.

### Western Blotting (Protein Expression)

Western blot analysis was carried out using an iBind Flex system (ThermoFisher Scientific) for primary and secondary antibody immunoblotting following a previously described method (Greenwood et al., 2022).

For cell lysate collection SKOV3-ip1 and SKOV3-TRip2 cells were seeded in 6-well plates for 24 h at a density of 1.25×10⁵ mL⁻¹ and 1.75×10⁵ mL⁻¹, respectively, in 2 mL of media. Lysates were collected in RIPA buffer, protein quantified by Pierce BCA assay and 20-30 mg loaded on a 10% polyacrylamide gel which was run at 200 V. Rabbit monoclonal primary antibodies against anti-ALDH1A1 (Cell Signaling Technology) was used at a concentration of 1:1000 and anti-rabbit IgG secondary antibody (1:200 dilution; Cell Signaling Technology) for cell lysates analysis. Actin was used as a loading control for all experiments (1:1000 dilution; Cell Signaling Technology). After antibody incubations membranes were washed 5× with tris-buffered saline with tween (TBST) and visualised using ECL regent (GE Healthcare), with images taken using an iBright CCD camera (Invitrogen). Images were always acquired within the linear range of the camera to prevent the overexposure of any blots.

### Generation of ALDH1A1 Knockout Cell Line (A549-KO-ALDH1A1 Cells) Using CRISPR.

A549-KO-ALDH1A1 cells were obtained using an ALDH1A1 human gene knockout kit (Cat #KN400723) according to manufacturer's instructions (Origene, Rockville, MD, USA). Briefly, 3×105 A549 cells were seeded into a 6-well plate and maintained for 24 hours at 37° C., 5% CO₂. Lipofectamine 3000 (Cat #L³⁰⁰⁰, Invitrogen) was mixed with 1 μg of gRNA vector (pCas-guide) and 1 μg of donor template in Opti-MEM and added to cells for 16 hours. The next day, the transfection medium was removed and replaced with fresh medium. Forty-eight hours post-transfection, medium was replaced with fresh medium containing 0.5 μg/mL of puromycin (Cat #A1113803) which caused 100% cell death in non-transfected control cells after 5 days. Cells resistant to puromycin were subjected to clonal selection by the limiting dilution method. Knockout of protein was confirmed by western blots.

### Generation of ALDH1A1 Overexpressing (HEK293-ALDH1A1) Cell Line Using Lentiviral Particles

HEK293 stably overexpressing ALDH1A1 were obtained by infecting cells with lentiviral particles containing the nucleotide sequences encoding for human ALDH1A1 (Origene RC200723L⁴V LentiORF particles (mGFP-tagged)). Briefly, 1×104 HEK-293 cells were seeded into a 24-well plate and maintained for 24 hours at 37° C., 5% CO₂. Cells were then infected with the lentiviral particles using a multiplicity of infection (MOI) of 10 in the presence of hexadimethrine bromide (6 μg/mL) (Sigma-Aldrich, St. Louis, MO, USA). The next day, medium was removed and replaced with fresh medium. At 36 h post-infection, medium was replaced with complete culture medium containing puromycin (1 μg/ml) which caused 100% cell death in non-transduced control cells after 5 days. Cells resistant to puromycin were subjected to clonal selection by the limiting dilution method. Overexpression of ALDH1A1 was confirmed by western blots and real-time PCR.

### In Vitro Characterization

### ALDH Activity Assay

ALDH substrate kinetics were measured using an ALDH activity colorimetric assay kit (Biovision) according to the manufacturer's instructions. 1 μg/well human recombinant ALDH1A1 (bio-techne), ALDH2 (abcam), or ALDH3A1 (ATGen Co., Ltd) were used. The assay was initiated by the addition of enzyme after all reagents were pre-warmed to 37° C. Plates were read in kinetic mode on a Multiskan FC plate reader (Thermo Scientific) at 450 nm over 40 min at 37° C.

### Cell Uptake/Efflux

For cell uptake studies SKOV3-ip1 and SKOV3-TRip2, A549-WT, A549-KO-ALDH1A1, HEK293-WT and HEK293-ALDH1A1 cells were plated in 6 well plates 24 h prior to the studies, as described above (Western Blotting). One mL of fresh media containing 0.37 MBq of PQM-H-001 and 6 KBq PQM-H-002 was added to each well for the desired incubation time. At 40 min, plates were placed on ice, washed three times with ice-cold PBS to remove exogenous radioactivity and lysed in RIPA buffer (500 mL; Fisher Scientific Ltd).

For cell efflux study, after 40 min incubation, every 20 min exogeneous radioactivity was removed from plates were washed with warm PBS and one mL warm fresh media added and incubated up to 160 min. After each desired incubation time plates were placed on ice, washed three times with ice-cold PBS to remove exogenous radioactivity and lysed in RIPA buffer (500 mL; Fisher Scientific Ltd).

For all cell uptake and efflux study decay-corrected radioactivity in lysates was determined on a gamma counter (300 mL of lysate; Wallac 1282 CompuGamma counter), with the remaining cell lysate centrifuged (21,000×g for 10 min at 4° C.) and the supernatant used to determine protein concentration following radioactive decay using a Pierce BCA assay. To quantify radiotracer uptake in cells, three standard solutions of the radioactivity-containing medium were counted on the gamma counter. Data were expressed as a percentage of total radioactivity administered to cells per mg of protein.

### Reference Compounds (PQM-C Compounds)

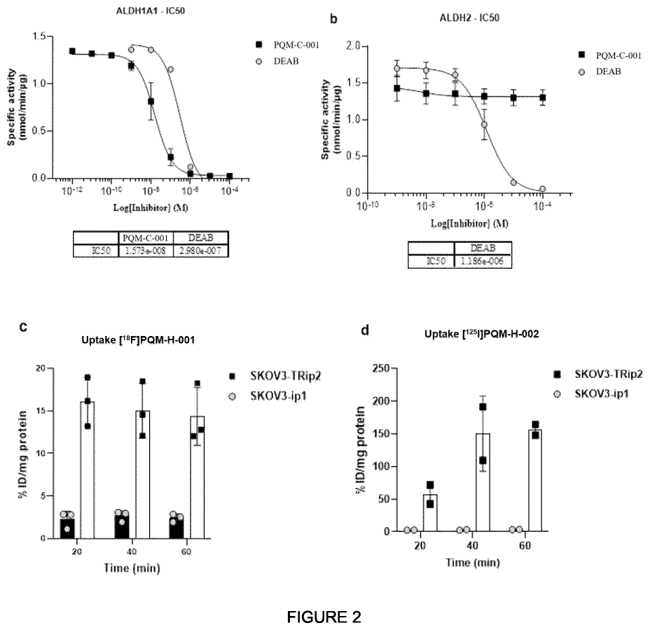

The in vitro potency of the cold, non-radioactive reference compound compared against two different ALDH isoforms, ALDH1A1 and ALDH2 (using the ALDH activity colorimetric assay Kit (Biovision)). Similar to previous findings (Yang, S. et al., 2018), the compound PQM-C-001 displayed nM potency and ALDH1A1 selectivity, out-competing the reference inhibitor, DEAB (FIG. 2a & 2b).

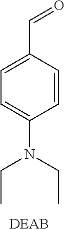

Other reference compounds (PQM-C) were compared against ALDH1A1 (using the ALDH activity colorimetric assay Kit (Biovision)). Results are shown in Table 1.

TABLE 1
IC₅₀ of PQM-C Reference Compounds against ALDH1A1

| Reference Compound | ALDH1A1 IC₅₀ |
|---|---|
| PQM-C-001 | B |
| PQM-C-002 | A |
| PQM-C-006 | B |
| PQM-C-007 | B |
| PQM-C-011 | B |
| PQM-C-012 | B |
| PQM-C-016 | B |
| PQM-C-017 | B |
| PQM-C-021 | A |
| PQM-C-022 | D |
| PQM-C-026 | A |
| PQM-C-027 | C |

A: ≤10 nM,
B: >10 nM to ≤100 nM,
C: >100 nM to ≤1 μM,
D: >1 μM to ≤10 μM,
E: >10 μM

### Example Compounds (PQM-H Compounds)

Next, the time-dependent uptake of the of the radiotracers PQM-H-001 ([¹⁸F]PQM-H-001) and PQM-H-002 ([¹²⁵I]PQM-H-002) was profiled in in drug-resistant SKOV3-Trip2 and drug-sensitive SKOV3-ip1 cell lines (Pereira, R., et al. 2019). Radiotracer retention was found to be 9 times higher (PQM-H-001) and 200 times higher (PQM-H-002) in the drug resistant versus the drug-sensitive cells (FIGS. 2c & 2d).

The following compounds were profiled in A549-WT (positive ALDH1A1) and A549-KO-ALDH1A1 (negative for ALDH1A1) human lung cancer cells as well as HEK293-WT (negative for ALDH1A1) and HEK293-ALDH1A1 (positive for ALDH1A1) human embryonic kidney cells. The fold difference in radiotracer retention for the ALDH1A1 positive cell lines as compared to the ALDH1A1 negative cell lines for each compound is shown in Table 2.

TABLE 2
Radiotracer retention data

| | A549-WT vs | HEK293-ALDH1A1 vs. |
|---|---|---|
| Compound | A549-KO-ALDH1A1 | HEK293-WT |
| PQM-H-002 | 40 | 56 |
| PQM-H-007 | 17 | 2 |
| PQM-H-017 | 16 | 6 |
| PQM-H-022 | 6 | — |

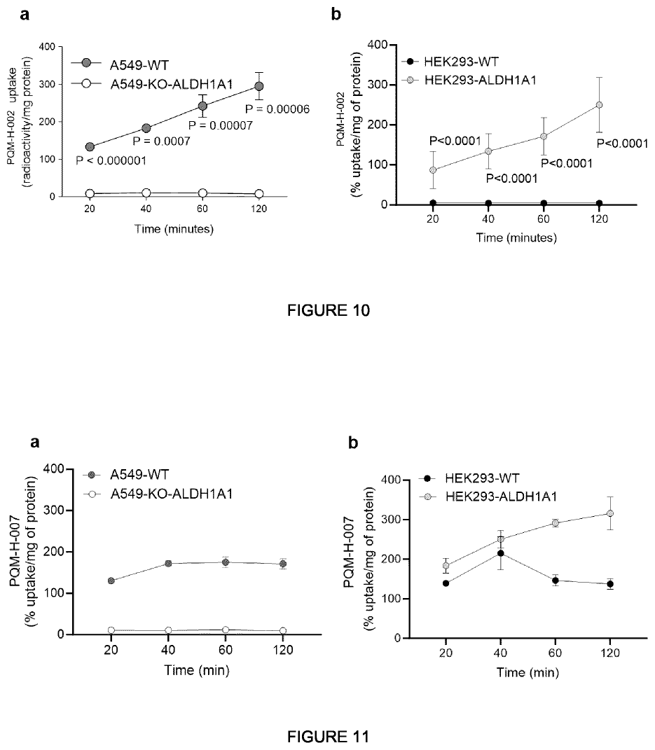

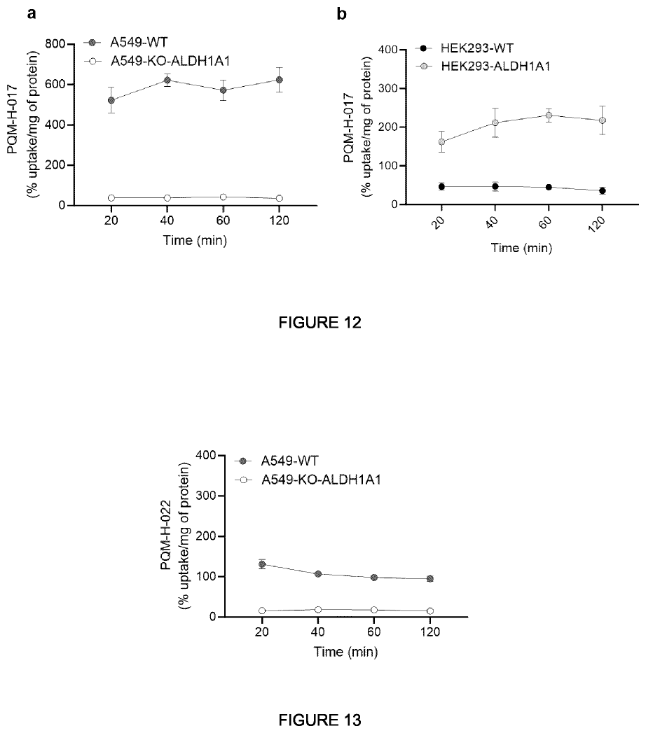

Cell-uptake data for PQM-H-002 is shown in FIGS. 10a and 10b; for PQM-H-007 is shown in FIGS. 11a and 11b; for PQM-H-017 is shown in FIGS. 12a and 12b; and for PQM-H-022 is shown in FIG. 13.

### In Vivo Characterization

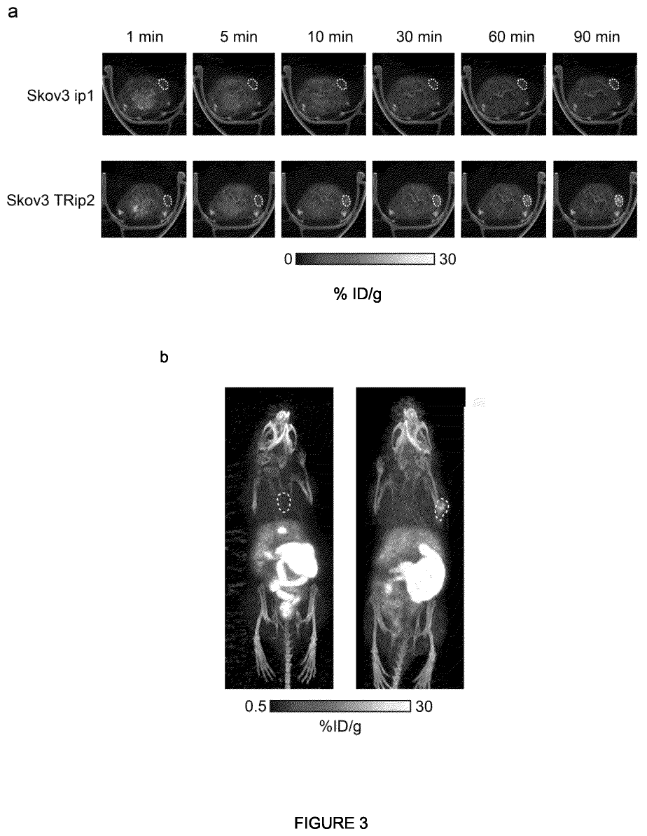

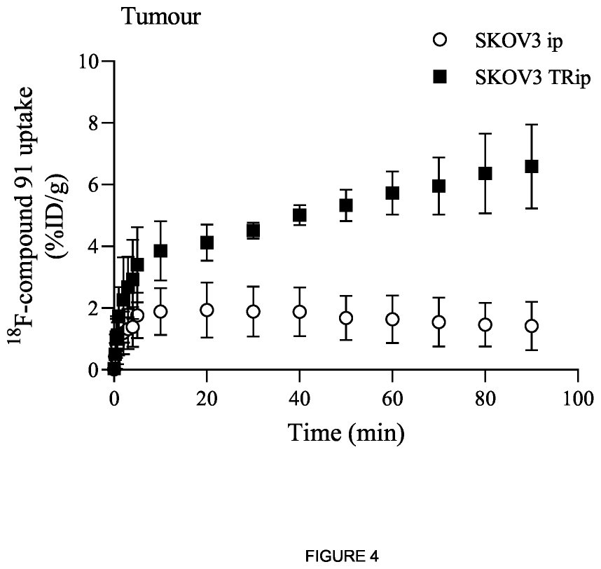

We have acquired compelling proof-of-concept data showing that PQM-H-001 can non-invasively identify drug resistant tumours in vivo (FIGS. 3a & 3b). Retention was found to be >5× higher in treatment resistant SKOV3-TRip2 subcutaneous tumours compared to SKOV3-ip1 tumours (FIG. 4).

In contrast, substrate-based imaging agents, are reportedly unable to distinguish between treatment resistant SKOV3-TRip2 and SKOV3-ip1 tumours in vivo (Pereira et al., 2022).

It is surprising and unexpected that the inhibitor based molecular imaging agents described herein provide improved sensitivity and selectivity for in vivo tumor characterization, as compared to a substrate based molecular imaging agent (Pereira et al., 2022).

### In Vivo Tumour Model and PET Imaging

All animal experiments were performed in accordance with the United Kingdom Home Office Animal (scientific procedures) Act 198. 2×106 SKOV3-ip1 and 4×106 and SKOV3-TRip2 cells were injected subcutaneously into female Balb/c nu/nu mice aged 6-9 weeks (Charles River Laboratories). Tumour dimensions were measured using an electronic calliper and the volume calculated using the following equation: volume=((π/6)×h×w×l), where h, w and l represent, height, width, and length, respectively. Imaging studies took place when tumour size reached approximately 150-250 mm³. Mice were anaesthetized with isoflurane (2.5% in oxygen) and maintained at 37° C. using an air-heated scanning bed. A tail vein cannula was inserted and a 3.7 MBq bolus of PQM-H-001 was administered in approximately 100 μL of PBS. Immediately following injection, 120 min PET acquisition was performed on a Mediso nanoScan PET/CT system. CT images were acquired for anatomical visualization and attenuation correction (480 projections; helical acquisition; 50 kVp; 300 ms exposure time). Reconstructed images (Tera-Tomo 3D reconstruction algorithm; 4 iterations; 6 subsets; 400-600 keV; 0.4 mm voxel size) were analysed using VivoQuant software (v. 2.5, Invicro Ltd.). Regions of interest (ROIs) were drawn manually on the CT images and the radioactivity concentration in each ROI expressed as a percentage of the injected dose per mL of tissue volume (% ID/mL).

### Ex Vivo Characterization

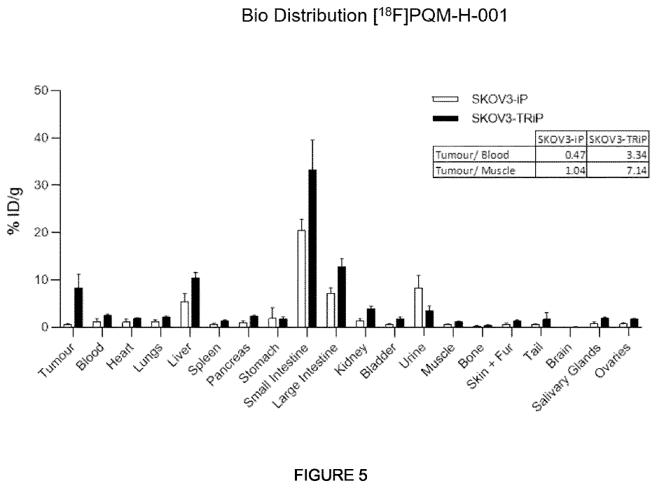

PQM-H-001 distribution was further evaluated ex vivo 90 min post radiotracer injection. Low radiotracer retention was observed in most healthy tissues, except the liver, with excretion occurring via the urinary and hepatobiliary tract (FIG. 5). Radiotracer retention was substantially increased in the drug-resistant tumours compared to drug-sensitive tumours, confirming the imaging data.

### Ex Vivo Biodistribution

Ex-vivo distribution of PQM-H-001 was performed after PET imaging (section: In Vivo Tumour Model and PET Imaging) by sacrificing all mice at time point 120 min post injection. Blood, tumour, and major organs and tissues were collected, wet-weighed, and counted using an automated y-counter (Wallac 1282 CompuGamma g counter). The concentrations of radioactivity in blood, tumor, and organs and tissues were determined as percentage injected of total radioactivity per gram (% ID/g).

The foregoing has described the principles, preferred embodiments, and modes of operation of the present invention. However, the invention should not be construed as limited to the particular embodiments discussed. Instead, the above-described embodiments should be regarded as illustrative rather than restrictive. It should be appreciated that variations may be made in those embodiments by workers skilled in the art without departing from the scope of the present invention.

### REFERENCES

(1) L. N. Abdullah, E. K.-H. Chow, Clin. Transl. Med. 2013, 2, 3.
(2) W. Ambroziak, R. Pietruszko, J. Biol. Chem. 1991, 266, 13011-13018.
(3) C. Anorma, J. Hedhli, T. E. Bearrood, N. W. Pino, S. H. Gardner, H. Inaba, P. Zhang, Y. F. Li, D. Feng, S. E. Dibrell, K. A. Kilian, L. W. Dobrucki, T. M. Fan and J. Chan, ACS Cent. Sci., 2018, 4, 1045-1055.
(4) W. J. Black, D. Stagos, S. A. Marchitti, D. W. Nebert, K. F. Tipton, A. Bairoch, V. Vasiliou, Pharmacogenet. Genomics 2009, 19, 893-902.
(5) Z. Duan, A. J. Feller, H. C. Toh, T. Makastorsis, M. V. Seiden, Gene, 1999, vol. 229, issue 1-2, pages 75-81.
(6) G. Duester, F. A. Mic, A. Molotkov, Chem.-Biol. Interact. 2003, 143-144, 201-210.
(7) H. E. Greenwood and T. H. Witney, J. Nucl. Med., 2021, 62, 1506-1510.
(8) H. E. Greenwood, R. Edwards, N. Koglin, M. Berndt, F. Baark, J. Kim, G. Firth, E. Khalil, A. Mueller, T. H. Witney, Theranostics, 2022; 12, 1921-1936.
(9) D. P. Hartley, J. A. Ruth, D. R. Petersen, Arch. Biochem. Biophys. 1995, 316, 197-205.
(10) C.-P. Huang, M.-F. Tsai, T.-H. Chang, W.-C. Tang, S.-Y. Chen, H.-H. Lai, T.-Y. Lin, J. C.-H. Yang, P.-C. Yang, J.-Y. Shih, S.-B. Lin, Cancer Lett. 2013, 328, 144-151.
(11) C. Kahlert, E. Gaitzsch, G. Steinert, C. Mogler, E. Herpel, M. Hoffmeister, L. Jansen, A. Benner, H. Brenner, J. Chang-Claude, N. Rahbari, T. Schmidt, F. Klupp, N. Grabe, B. Lahrmann, M. Koch, N. Halama, M. Bechler, J. Weitz, Ann. Surg. Oncol. 2012, 19, 4193-4201.
(12) C. N. Landen, B. Goodman, A. A. Katre, A. D. Steg, A. M. Nick, R. L. Stone, L. D. Miller, P. V. Mejia, N. B. Jennings, D. M. Gershenson, R. C. Bast, R. L. Coleman, G. Lopez-Berestein, A. K. Sood, Mol. Cancer Ther. 2010, 9, 3186-3199.
(13) T. Li, Y. Su, Y. Mei, Q. Leng, B. Leng, Z. Liu, S. A. Stass, F. Jiang, Lab. Invest. 2010, 90, 234.
(14) S. Liu, C. Liu, X. Min, Y. Ji, N. Wang, D. Liu, J. Cai, K. Li, PLoS One 2013, 8, e81050.
(15) Y. Liu, D. I. Lv, J. Duan, S. I. Xu, J. Zhang, X. Yang, X. Zhang, Y. Cu, X. Bian, S. Yu, BMC Cancer 2014, 14, 444.
(16) S. Maity, C. M. Sadlowski, J. M. George Lin, C. H. Chen, L. H. Peng, E. S. Lee, G. K. Vegesna, C. Lee, S. H. Kim, D. Mochly-Rosen, S. Kumar and N. Murthy, Chem. Sci., 2017, 8, 7143-7151.
(17) P. Marcato, C. A. Dean, C. A. Giacomantonio, P. W. K. Lee, Cell Cycle 2011, 10, 1378-1384.
(18) I. Minn, H. F. Wang, R. C. Mease, Y. Byun, X. Yang, J. L. Wang, S. D. Leach and M. G. Pomper, Nat. Commun., 2014, 5, 3662.
(19) K. Nakahata, S. Uehara, S. Nishikawa, M. Kawatsu, M. Zenitani, T. Oue, H. Okuyama, PLoS One 2015, 10, e0125454.
(20) A. Okamoto et al., Identification of Breast Cancer Stem Cells Using a Newly Developed Long-acting Fluorescence Probe, C5S-A, Targeting ALDH1A1, Anticancer Res., 2022, 42, 1199-1205.
(21) R. Pereira, T. Gendron, C. Sanghera, H. E. Greenwood, J. Newcombe, P. N. McCormick, K. Sander, M. Topf, E. Arstad and T. H. Witney, Chem. —Eur. J., 2019, 25, 2345-2351.
(22) R. Pereira, R. L. Flaherty, R. S. Edwards, H. E. Greenwood, H. J. Shuhendler and T. H. Witney, RSC Chem. Biol., 2022, 3, 561-570.
(23) D. Raha, T. R. Wilson, J. Peng, D. Peterson, P. Yue, M. Evangelista, C. Wilson, M. Merchant, J. Settleman, Cancer Res. 2014, 74, 3579-3590.
(24) Shen, Y., Systematic prediction of drug resistance caused by transporter genes in cancer cells. Sci Rep, 2021, 11, 7400.
(25) S. Singh, C. Brocker, V. Koppaka, Y. Chen, B. C. Jackson, A. Matsumoto, D. C. Thompson, V. Vasiliou, Free Radical Biol. Med. 2013, 56, 89-101.
(26) N. E. Sladek, J. Biochem. Mol. Toxicol. 2003, 17, 7-23.
(27) D. Ucar, C. R. Cogle, J. R. Zucali, B. Ostmark, E. W. Scott, R. Zori, B. A. Gray and J. S. Moreb, Chem. —Biol. Interact., 2009, 178, 48-55.
(28) G. Vaidyanathan, H. J. Song, D. Affleck, D. L. McDougald, R. W. Storms, M. R. Zalutsky and B. B. Chin, Nucl. Med. Biol., 2009, 36, 919-929.
(29) V. Vasiliou, A. Pappa, D. R. Petersen, Chem.—Biol. Interact. 2000, 129, 1-19.
(30) M. Volm and T. Efferth, Front Oncol. 2015, 17, 5, 282.
(31) A. Woolston, L. J. Barber, B. Griffiths, O. Pich, N. Lopez-Bigas, N. Matthews, S. Rao, D. Watkins, I. Chau, N. Starling, D. Cunningham, M. Gerlinger, Nat Ecol Evol, 2021, 5, 7, 1024-1032.
(32) A. Yagishita, T. Ueno, K. Tsuchihara, Y. Urano, Amino BODIPY-Based Blue Fluorescent Probes for Aldehyde Dehydrogenase 1-Expressing Cells, Bioconjugate Chemistry, 2021, 32(2), 234-238
(33) S. Yang, N. Martinez, A. Yasgar, C. Danchik, C. Johansson, Y. Wang, B. Baljinnyam, A. Wang, X. Xu, P. Shah, D. Cheff, X. Wang, J. Roth, M. Lal-Nag, J. Dunford, U. Oppermann, V. Vasiliou, A. Simeonov, A. Jadhav, D. Maloney, J. Med. Chem., 2018, 61, 4883-4903.
(34) L. H. Yuen, N. S. Saxena, H. S. Park, K. Weinberg, E. T. Kool, ACS Chem. Biol., 2016, 11, 8, 2312-2319.

1. A radiolabelled compound according to formula (I): 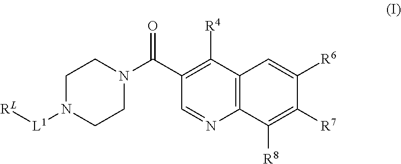
or a pharmaceutically acceptable salt, hydrate, or solvate thereof;
wherein:
—R⁴ 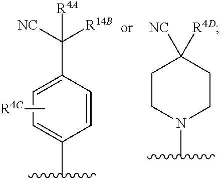
—R^{4A} and —R^{4B} taken together with the carbon atom to which they are attached form a C₃₋₅ cycloalkyl; or —R^{4A} and —R^{4B} are each independently C₁₋₃ alkyl;
—R^{4C} is —H, —F, or —R^{X4C};
—R^{4D} is phenyl, optionally substituted with:
a) one group —R^{X4D}; and/or
b) one or more —F;
-L¹- is independently selected from —S(O)₂— and —C(O)—;
—R^{L} is independently selected from C₁₋₆ alkyl and cyclopropyl;
—R⁶ is independently selected from —H, —F, —I, —R^{X6}, and —NO₂;
—R⁷ is independently selected from —H, —F, —I, —R^{X7}, and —NO₂;
—R⁸ is independently selected from —H, —F, —I, —R^{X8}, and —NO₂;
—R^{X4C} is a radioisotope selected from ¹⁸F, ¹²³I, ¹²⁴I, ¹²⁵I, ¹³¹I, and ²¹¹At;
—R^{X4D} is a radioisotope selected from ¹⁸F, ¹²³I, ¹²⁴I, ¹²⁵I, ¹³¹I, and ²¹¹At;
—R^{X6} is a radioisotope selected from ¹⁸F, ¹²³I, ¹²⁴I, ¹²⁵I, ¹³¹I, and ²¹¹At;
—R^{X7} is a radioisotope selected from ¹⁸F, ¹²³I, ¹²⁴I, ¹²⁵I, ¹³¹I, and ²¹¹At; and
—R^{X8} is a radioisotope selected from ¹⁸F, ¹²³I, ¹²⁴I, ¹²⁵I, ¹³¹I, and ²¹¹At;
wherein, the compound comprises only one group selected from —R^{X4C}, —R^{X4D}, —R^{X6}, —R^{X7}, and —R^{X8}; and at least one of —R⁶, —R⁷, and —R⁸ is —H.

2. A radiolabelled compound according to claim 1, which is a compound of formula (I): 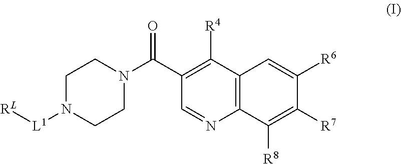
or a pharmaceutically acceptable salt, hydrate, or solvate thereof;
wherein:
—R⁴ is 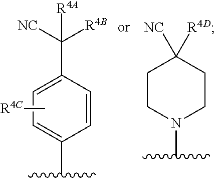
—R^{4A} and —R^{4B} taken together with the carbon atom to which they are attached form a C₃₋₅ cycloalkyl; or —R^{4A} and —R^{4B} are each independently C₁₋₃ alkyl;
—R^{4C} is —H or —F;
—R^{4D} is phenyl, optionally substituted with one of more —F;
-L¹- is independently selected from —S(O)₂— and —C(O)—;
—R^{L} is independently selected from C₁₋₆ alkyl and cyclopropyl;
—R⁶ is independently selected from —H, —F, —I, a radioisotope, and —NO₂;
—R⁷ is independently selected from —H, —F, —I, a radioisotope, and —NO₂;
—R⁸ is independently selected from —H, —F, —I, a radioisotope, and —NO₂;
wherein, one of —R⁶, —R⁷, and —R⁸ is a radioisotope, and the others of —R⁶, —R⁷, and —R⁸ are not radioisotopes; and at least one of —R⁶, —R⁷, and —R⁸ is —H;
wherein the radioisotope is selected from ¹⁸F, ¹²³I, ¹²⁴I, ¹²⁵I, ¹³¹I, and ²¹¹At.

3. The compound according to claim 1, or a pharmaceutically acceptable salt, hydrate, or solvate thereof, wherein:
—R⁴ is

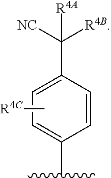

4. The compound according to claim 1, or a pharmaceutically acceptable salt, hydrate, or solvate thereof, wherein:
—R^{4A} and —R^{4B} taken together with the carbon atom to which they are attached form cyclopropyl.

5. The compound according to claim 1, or a pharmaceutically acceptable salt, hydrate, or solvate thereof, wherein:
—R^{4C} is —H.

6. The compound according to claim 1, or a pharmaceutically acceptable salt, hydrate, or solvate thereof, wherein:
-L¹- is —S(O)₂—.

7. The compound according to claim 1, or a pharmaceutically acceptable salt, hydrate, or solvate thereof, wherein:
—R^{L} is methyl.

8. The compound according to claim 1, or a pharmaceutically acceptable salt or solvate thereof, wherein:
—R^{X4C}, if present, is a radioisotope that is ¹⁸F;
—R^{X4D}, if present, is a radioisotope that is ¹⁸F;
—R^{X6}, if present, is a radioisotope selected from ¹⁸F, ¹²³I, ¹²⁴I, ¹²⁵I, and ¹³¹I;
—R^{X7}, if present, is a radioisotope selected from ¹⁸F, ¹²³I, ¹²⁴I, ¹²⁵I, and ¹³¹I; and
—R^{X8}, if present, is a radioisotope selected from ¹⁸F, ¹²³I, ¹²⁴I, ¹²⁵I, and ¹³¹I.

9. The compound according to claim 1, or a pharmaceutically acceptable salt, hydrate, or solvate thereof, wherein:
—R⁶ is the radioisotope;
—R⁷ is —H or —NO₂;
—R⁸ is —H or —NO₂; and
the radioisotope is ¹⁸F.

10. The compound according to claim 1, or a pharmaceutically acceptable salt, hydrate, or solvate thereof, wherein:
—R⁶ is the radioisotope;
—R⁷ is —H or —NO₂;
—R⁸ is —H or —NO₂; and
the radioisotope is ¹²⁵I.

11. The compound according to claim 1, or a pharmaceutically acceptable salt, hydrate, or solvate thereof, wherein:
—R⁸ is —H.

12. A compound according to claim 1 which is selected from:
1-(4-(6-(fluoro-¹⁸F)-3-(4-(methylsulfonyl)piperazine-1-carbonyl)quinolin-4-yl)phenyl)cyclopropane-1-carbonitrile;
1-(4-(6-(iodo-¹²⁵I)-3-(4-(methylsulfonyl)piperazine-1-carbonyl)quinolin-4-yl)phenyl)cyclopropane-1-carbonitrile;
1-(4-(6-(iodo-¹³¹I)-3-(4-(methylsulfonyl)piperazine-1-carbonyl)quinolin-4-yl)phenyl)cyclopropane-1-carbonitrile;
1-(4-(6-(iodo-¹²³I)-3-(4-(methylsulfonyl)piperazine-1-carbonyl)quinolin-4-yl)phenyl)cyclopropane-1-carbonitrile;
1-(4-(6-(iodo-¹²¹I)-3-(4-(methylsulfonyl)piperazine-1-carbonyl)quinolin-4-yl)phenyl)cyclopropane-1-carbonitrile;
1-(4-(6-(fluoro-¹⁸F)-3-(4-(methylsulfonyl)piperazine-1-carbonyl)-8-nitroquinolin-4-yl)phenyl)cyclopropane-1-carbonitrile;
1-(4-(6-(iodo-¹²⁵I)-3-(4-(methylsulfonyl)piperazine-1-carbonyl)-8-nitroquinolin-4-yl)phenyl)cyclopropane-1-carbonitrile;
1-(4-(6-(iodo-¹³¹I)-3-(4-(methylsulfonyl)piperazine-1-carbonyl)-8-nitroquinolin-4-yl)phenyl)cyclopropane-1-carbonitrile;
1-(4-(6-(iodo-¹²³I)-3-(4-(methylsulfonyl)piperazine-1-carbonyl)-8-nitroquinolin-4-yl)phenyl)cyclopropane-1-carbonitrile;
1-(4-(6-(iodo-¹²⁴I)-3-(4-(methylsulfonyl)piperazine-1-carbonyl)-8-nitroquinolin-4-yl)phenyl)cyclopropane-1-carbonitrile;
1-(4-(7-(fluoro-¹⁸F)-3-(4-(methylsulfonyl)piperazine-1-carbonyl)quinolin-4-yl)phenyl)cyclopropane-1-carbonitrile;
1-(4-(7-(iodo-¹²⁵I)-3-(4-(methylsulfonyl)piperazine-1-carbonyl)quinolin-4-yl)phenyl)cyclopropane-1-carbonitrile;
1-(4-(7-(iodo-¹³¹I)-3-(4-(methylsulfonyl)piperazine-1-carbonyl)quinolin-4-yl)phenyl)cyclopropane-1-carbonitrile;
1-(4-(7-(iodo-¹²³I)-3-(4-(methylsulfonyl)piperazine-1-carbonyl)quinolin-4-yl)phenyl)cyclopropane-1-carbonitrile;
1-(4-(7-(iodo-¹²⁴I)-3-(4-(methylsulfonyl)piperazine-1-carbonyl)quinolin-4-yl)phenyl)cyclopropane-1-carbonitrile;
1-(4-(8-(fluoro-¹⁸F)-3-(4-(methylsulfonyl)piperazine-1-carbonyl)quinolin-4-yl)phenyl)cyclopropane-1-carbonitrile;
1-(4-(8-(fluoro-¹²⁵I)-3-(4-(methylsulfonyl)piperazine-1-carbonyl)quinolin-4-yl)phenyl)cyclopropane-1-carbonitrile;
1-(4-(8-(fluoro-¹³¹I)-3-(4-(methylsulfonyl)piperazine-1-carbonyl)quinolin-4-yl)phenyl)cyclopropane-1-carbonitrile;
1-(4-(8-(fluoro-¹²³I)-3-(4-(methylsulfonyl)piperazine-1-carbonyl)quinolin-4-yl)phenyl)cyclopropane-1-carbonitrile;
1-(4-(8-(fluoro-¹²⁴I)-3-(4-(methylsulfonyl)piperazine-1-carbonyl)quinolin-4-yl)phenyl)cyclopropane-1-carbonitrile;
1-(2-(fluoro-¹⁸F)-4-(3-(4-(methylsulfonyl)piperazine-1-carbonyl)quinolin-4-yl)phenyl)cyclopropane-1-carbonitrile;
1-(2-(fluoro-¹²⁵I)-4-(3-(4-(methylsulfonyl)piperazine-1-carbonyl)quinolin-4-yl)phenyl)cyclopropane-1-carbonitrile;
1-(2-(fluoro-¹³¹I)-4-(3-(4-(methylsulfonyl)piperazine-1-carbonyl)quinolin-4-yl)phenyl)cyclopropane-1-carbonitrile;
1-(2-(fluoro-¹²³I)-4-(3-(4-(methylsulfonyl)piperazine-1-carbonyl)quinolin-4-yl)phenyl)cyclopropane-1-carbonitrile;
1-(2-(fluoro-¹²⁴I)-4-(3-(4-(methylsulfonyl)piperazine-1-carbonyl)quinolin-4-yl)phenyl)cyclopropane-1-carbonitrile;
4-(4-(fluoro-¹⁸F)phenyl)-1-(3-(4-(methylsulfonyl)piperazine-1-carbonyl)quinolin-4-yl)piperidine-4-carbonitrile;
4-(4-(fluoro-¹²⁵I)phenyl)-1-(3-(4-(methylsulfonyl)piperazine-1-carbonyl)quinolin-4-yl)piperidine-4-carbonitrile;
4-(4-(fluoro-¹³¹I)phenyl)-1-(3-(4-(methylsulfonyl)piperazine-1-carbonyl)quinolin-4-yl)piperidine-4-carbonitrile;
4-(4-(fluoro-¹²³I)phenyl)-1-(3-(4-(methylsulfonyl)piperazine-1-carbonyl)quinolin-4-yl)piperidine-4-carbonitrile; and
4-(4-(fluoro-¹²¹I)phenyl)-1-(3-(4-(methylsulfonyl)piperazine-1-carbonyl)quinolin-4-yl)piperidine-4-carbonitrile;
or a pharmaceutically acceptable salts, hydrates, and solvates thereof.

13. A compound according to claim 1, selected from compounds of the following formulae and pharmaceutically acceptable salts, hydrates, and solvates thereof:

| Cmpd No. | Structure |
|---|---|
| PQM-001 | 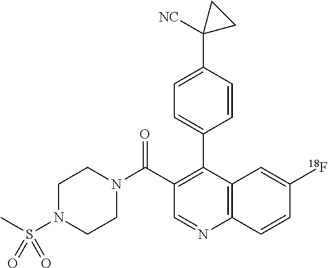 [SMILES: CS(=O)(=O)N1CCN(C(=O)c2cnc3ccc([18F])cc3c2-c2ccc(C3(C#N)CC3)cc2)CC1] |
| PQM-002 | 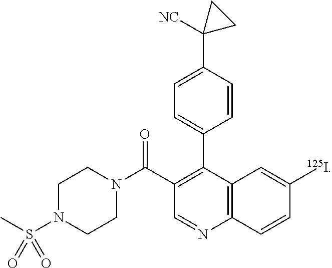 |

14. A compound according to formula (Ia): 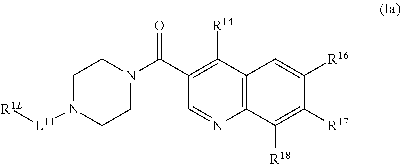
or a salt, hydrate, or solvate thereof;
wherein:
—R¹⁴ is 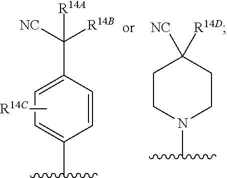
—R^{14A} and —R^{14B} taken together with the carbon atom to which they are attached form a C₃₋₅ cycloalkyl; or —R^{14A} and —R^{14B} are each independently C₁₋₃ alkyl;
—R^{14C} is —H, —F, or —R^{X14C};
—R^{14D} is phenyl, optionally substituted with:
a) one group —R^{X14D}; and/or
b) one or more —F;
-L¹¹- is independently selected from —S(O)₂— and —C(O)—;
—R^{1L} is independently selected from C₁₋₆ alkyl and cyclopropyl;
—R¹⁶ is independently selected from —H, —F, —I, —R^{X16}, and —NO₂;
—R¹⁷ is independently selected from —H, —F, —I, —R^{X17}, and —NO₂;
—R¹⁸ is independently selected from —H, —F, —I, —R^{X18}, and —NO₂;
each of —R^{X14C}, —R^{X14D}, —R^{X16}, —R^{X17}, and —R^{X18} is, independently, a leaving group;
the compound comprises only one group selected from —R^{X14C}, —R^{X14D}, —R^{X16}, —R^{X17}, and —R^{X18};
and at least one of —R¹⁶, —R¹⁷, and —R¹⁸ is —H.

15. The compound according to claim 14 which is a compound of formula (Ia): 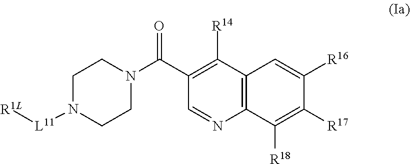
or a salt, hydrate, or solvate thereof;
wherein:
—R¹⁴ is 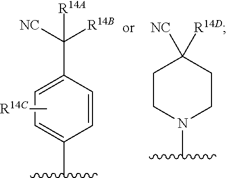
—R^{14A} and —R^{14B} taken together with the carbon atom to which they are attached form a C₃₋₅ cycloalkyl; or —R^{14A} and —R^{14B} are each independently C₁₋₃ alkyl;
—R^{14C} is —H or —F;
—R^{14D} is phenyl, optionally substituted with one of more —F;
L¹¹- is independently selected from —S(O)₂— and —C(O)—;
—R^{1L} is independently selected from C₁₋₆ alkyl and cyclopropyl;
—R¹⁶ is independently selected from —H, —F, —I, a leaving group, and —NO₂;
—R¹⁷ is independently selected from —H, —F, —I, a leaving group, and —NO₂;
—R¹⁸ is independently selected from —H, —F, —I, a leaving group, and —NO₂;
wherein, one of —R¹⁶, —R¹⁷, and —R¹⁸ is a leaving group, and the others of —R¹⁶, —R¹⁷, and —R¹⁸ are not leaving groups; and at least one of —R¹⁶, —R¹⁷, and —R¹⁸ is —H.

16. The compound according to claim 14, or a salt, hydrate, or solvate thereof, wherein:
—R¹⁴ is

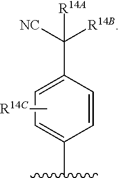

17. The compound according to claim 14, or a salt, hydrate, or solvate thereof, wherein:
—R^{14A} and —R^{14B} taken together with the carbon atom to which they are attached form cyclopropyl.

18. The compound according to claim 14, or a salt, hydrate, or solvate thereof, wherein:
—R^{14C} is —H.

19. The compound according to claim 14, or a salt, hydrate, or solvate thereof, wherein:
-L¹¹- is —S(O)₂—.

20. The compound according to claim 14, or a salt, hydrate, or solvate thereof, wherein:
—R^{1L} is methyl.

21. The compound according to claim 14, or a salt, hydrate, or solvate thereof, wherein:
—R¹⁶ is a leaving group;
—R¹⁷ is —H or —NO₂; and
—R¹⁸ is —H or —NO₂.

22. The compound according to claim 14, or a salt, hydrate, or solvate thereof, wherein:
the leaving group is selected from boronic acid esters, boronic acid, alkylsulfonates, haloalkylsulfonates, arylsulfonates, trialkylamines, organostannanes, aryliodonium salts, and arylsulfonium salts.

23. A compound according to claim 1 which is selected from:
1-[4-[3-(4-methylsulfonylpiperazine-1-carbonyl)-6-(4,4,5,5-tetramethyl-1,3,2-dioxaborolan-2-yl)-4-quinolyl]phenyl]cyclopropanecarbonitrile;
1-[4-[3-(4-methylsulfonylpiperazine-1-carbonyl)-8-nitro-6-(4,4,5,5-tetramethyl-1,3,2-dioxaborolan-2-yl)-4-quinolyl]phenyl]cyclopropanecarbonitrile;
1-[4-[3-(4-methylsulfonylpiperazine-1-carbonyl)-7-(4,4,5,5-tetramethyl-1,3,2-dioxaborolan-2-yl)-4-quinolyl]phenyl]cyclopropanecarbonitrile;
[4-[4-(1-cyanocyclopropyl)phenyl]-3-(4-methylsulfonylpiperazine-1-carbonyl)-8-quinolyl]boronic acid;
1-[4-[3-(4-methylsulfonylpiperazine-1-carbonyl)-4-quinolyl]-2-(4,4,5,5-tetramethyl-1,3,2-dioxaborolan-2-yl)phenyl]cyclopropanecarbonitrile; and
1-[3-(4-methylsulfonylpiperazine-1-carbonyl)-4-quinolyl]-4-[4-(4,4,5,5-tetramethyl-1,3,2-dioxaborolan-2-yl)phenyl]piperidine-4-carbonitrile;
or a salt, hydrate, or solvate thereof.

24. A compound according to claim 14, that is:

| Cmpd No. | Structure |
|---|---|
| PQM-P-001 | 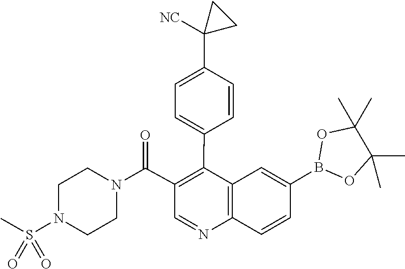 |

or a salt, hydrate, or solvate thereof.

25. (canceled)

26. A process for preparing a compound according to claim 1, or a pharmaceutically acceptable salt, hydrate, or solvate thereof,
comprising the step of contacting:
a compound according to formula (Ia): 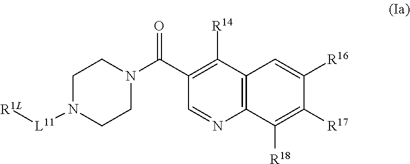
or a salt, hydrate, or solvate thereof;
wherein:
—R¹⁴ is 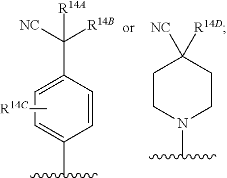
—R^{14A} and —R^{14B} taken together with the carbon atom to which they are attached form a C₃₋₅ cycloalkyl; or —R^{14A} and —R^{14B} are each independently C₁₋₃ alkyl;
—R^{14C} is —H, —F, or —R^{X14C};
—R^{14D} is phenyl, optionally substituted with:
a) one group —R^{X14D}; and/or
b) one or more —F;
-L¹¹- is independently selected from —S(O)₂— and —C(O)—;
—R^{1L} is independently selected from C₁₋₆ alkyl and cyclopropyl;
—R¹⁶ is independently selected from —H, —F, —I, —R^{X16}, and —NO₂;
—R¹⁷ is independently selected from —H, —F, —I, —R^{X17}, and —NO₂;
—R¹⁸ is independently selected from —H, —F, —I, —R^{X18}, and —NO₂;
each of —R^{X14C}, —R^{X14D}, —R^{X16}, —R^{X17}, and —R^{X18} is, independently, a leaving group;
the compound comprises only one group selected from —R^{X4C}, —RX^{14D}, —RX¹⁶, —R^{X17}, and —R^{X18};
and at least one of —R¹⁶, —R¹⁷, and —R¹⁸ is —H, with
a source of radioisotope;
under conditions to replace a leaving group on the compound, salt, hydrate, or solvate according to formula (Ia), with a radioisotope from the source of radioisotope.

27. (canceled)

28. A pharmaceutical composition comprising a compound according to claim 1, or a pharmaceutically acceptable salt, hydrate, or solvate thereof; and a pharmaceutically acceptable carrier, diluent, or excipient.

29. A method of diagnosis or therapy comprising administering to a subject a compound according to claim 1, or a pharmaceutically acceptable salt, hydrate, or solvate thereof.

30. A method of diagnosis or treatment of cancer comprising administering to a subject a compound according to claim 1, or a pharmaceutically acceptable salt, hydrate, or solvate thereof.

31. A method of medical imaging comprising the steps:
1) administering to a subject:
a compound according to claim 1, or a pharmaceutically acceptable salt, hydrate, or solvate thereof;
then
2) imaging the subject by PET, SPECT or scintigraphy.
